(19) United States
(12) Patent Application Publication
(10) Pub. No.: US 2026/0007729 A1
(43) Pub. Date: Jan. 8, 2026

(54) IMMUNIZATION AGAINST VIRAL INFECTIONS DISEASE(S)

(71) Applicant: IMMUNEO Therapeutics GmbH, Brandenburg an der Havel (DE)

(72) Inventors: Wolfgang SCHÖNHARTING, Bad Wiessee (DE); Mari Carmen MARTOS CONTRERAS, München (DE); Meik KUNZ, Amberg (DE)

(21) Appl. No.: 19/104,219

(22) PCT Filed: Aug. 17, 2023
(86) PCT No.: PCT/EP2023/072715

§ 371 Date: Feb. 16, 2025

(30) Foreign Application Priority Data
Aug. 17, 2022 (LU) LU102995
Feb. 28, 2023 (LU) LU103079

(51) Int. Cl. A61K 39/00; A61P 37/04; C12Q 1/70

(52) U.S. Cl. CPC A61K 39/0005; A61P 37/04; C12Q 1/701; A61K 2039/58; C12Q 2600/156

(57) ABSTRACT

The present invention relates to a pharmaceutical composition to prevent or treat viral infection(s), particularly to a combination agent/composition comprising of at least two and most particularly up to 12 different antigen polypeptides corresponding to HLA antigen peptides matching viral epitopes, for immunization against at least one, preferably two, particularly preferably up to 4 viral infectious diseases, a combination preparation (or parts thereof), a method for determining/identifying at least one HLA antigen peptide corresponding to the MHC class I complexes and/or antigen polypeptide for use in the pharmaceutical composition and a method for preparing a pharmaceutical composition comprising at least one antigen polypeptide according to the invention.

### TECHNICAL FIELD OF THE INVENTION

This disclosure relates to the field of amino acids to prevent or treat viral infection(s). In particular, the present invention relates to a pharmaceutical composition/formulation, particularly to a combination agent/composition comprising of at least two and most particularly up to 12 different antigen polypeptides corresponding to HLA antigen peptides matching viral epitopes, for immunization against at least one, preferably two, particularly preferably up to 4 viral infectious diseases, a combination preparation (or parts thereof), a method for determining/identifying at least one HLA antigen peptide corresponding to the MHC class I complexes and/or antigen polypeptide for use in the pharmaceutical composition and a method for preparing a pharmaceutical composition comprising at least one antigen polypeptide according to the invention.

### State of the Art/Background

Viruses are infectious organic structures which, as virions (virion=single virus particle), spread outside cells (extracellularly) by transmission, but as viruses can only replicate within a suitable host cell (intracellularly). Viruses do not exhibit independent replication or metabolism, so they rely on the metabolism of a host cell. Viruses infect cells of eukaryotes (plants, fungi, and animals including humans) as well as prokaryotes (bacteria and archaea). Virions are particles that contain nucleic acids and usually have an enclosing protein capsule (capsid). Some virions additionally possess an envelope by a bio membrane whose lipid bilayer is interspersed with viral membrane proteins, which is called the viral envelope. The replication cycle of a virus begins when a virion attaches to a surface protein on a host cell that is used by the virus as a receptor. After ingestion, a virion must first be freed from its envelopes before replication. The genetic material of the virus, its nucleic acid, is then replicated in the host cell, and the envelope proteins and possibly other components of the virions are also synthesized by the host cell using the genes of the viral genome. In this way, new viruses can be formed in the cell and released as virions, either by dissolving the cell membrane or by being shed, taking parts of the host cell membrane with them as part of the viral envelope. Human pathogenic viruses are transmitted, for example, through the air by droplet infection or through contaminated surfaces by smear infection. Coronaviruses (Orthocoronavirinae) are a group of related RNA viruses which cause respiratory tract infections.

Severe acute respiratory syndrome coronavirus type 2 (SARS-COV-2) is a strain of the subgenus sarbecovirus, of the genus of betacoronaviruses and was responsible for the global pandemic outbreak in 2019 and closely related to SARS-COV-1 virus which was responsible for the 2002-2004 SARS outbreak. These outbreaks lead to immense loss of human life and economical damage.

The virus still poses a drastic challenge to healthcare worldwide, as new and more contagious variants emerge due to mutation of the original Wuhan wild type (Hu-1). Some particularly worrisome strains are tracked by the World Health Organization (WHO) as variants of concern (VOC) such as the Alpha (B.1.1.7), Beta (B.1.351), Gamma (P. 1), Delta (B.1.617.2) and Omicron Variant (B.1.1.529). These variants commonly feature mutations especially on the spike(S) glycoprotein, also known as spike protein, which is targeted by many vaccines developed against the wild type and thus impeding immunization efforts worldwide.

The immune system continuously monitors the body for the presence of viral infections and degenerate or infected cells by checking whether cells present endogenous or foreign proteins, and for the presence of the MHC-(I-III) major histocompatibility complexes. MHC protein complexes are endogenous proteins that bind endogenous or exogenous peptides that are approximately 8-10 (MHC I) or 13-18 amino acids (MHC II) in length. Antigens on the surface of body cells serve to identify the body's own cells. This is the origin of the name HLA system (Human Leucocyte Antigen) for the regulatory system of human immune defense, whose most important component is the MHC. The HLA system in other species is known as the major histocompatibility complex (MHC) and is found in all vertebrates.

There are two types of MHCs, class I MHC molecules (herein also “class I MHC complexes” or “class I HLA complexes”) and class II MHC molecules (herein also “class II MHC complexes” or “class II HLA complexes”). Both show up on the cell surface of all nucleated cells in the bodies of gnathostomata. Class I MHC molecules, with (viral) antigens (peptides) presented on them, are expressed on the cell surface of all nucleated cells and are recognized by CD8+ T cells (also called killer T cells or cytotoxic T cells). The non-presenting HLA class I molecule (MHC-I) is recognized by NK cells. A missing MHC-I complex on the surface of an (e.g. infected) cell causes activation of the NK cell and killing of the (infected) cell. The immune system continuously monitors the body for the presence of viral infections and degenerate (infected) cells by checking whether cells present endogenous or foreign proteins and for the presence of MHC-I protein complexes. The peptides presented reflect the proteins synthesized in the cells. Cytotoxic T lymphocytes (CD8+ T cells) are selected so that they generally do not bind with their T cell receptor (TCR) to cells presenting a peptide derived from an endogenous protein. MHC class II (MHC-II) molecules are mainly exposed on the cell surface of antigen-presenting cells (including macrophages) and recognized by CD4+ T cells (also known as T helper cells), which function as a co-receptor of the T cell receptor (TCR) and play an important role in the antigen detection by the latter.

With the identification of the genomes of pathogens, for example Coronaviruses, such as SARS-COV-2 (a single-stranded RNA virus), and/or Orthomyxoviridae, a virus family containing Alphainfluenzavirus, Betainfluenzavirus, Gammainfluenzavirus, Deltainfluenzavirus, Isavirus, Thogotovirus, and Quaranjavirus (negative-sense RNA viruses) and/or poliovirus (from the family of Picornaviridae with single-stranded positive-sense RNA), and/or Rotavirus (from the family of Reoviridae with double stranded RNA viruses), and/or tick-borne encephalitis virus (TBEV, from the family of Flaviviruses with a positive-strand RNA), and/or human papillomavirus (HPV, from the Papillomaviridae family of DNA viruses), their protein repertoire can be determined by DNA/RNA sequencing. This involves the use of bioinformatics processes that recognize regions for epitopes in these proteins. Since T and B cells identify these regions, vaccines can be developed on this basis.

There are existing combination immunization strategies against multiple viral infectious diseases as single preparation vaccinations, such as Comvax (which combines Hib and Hep B), Twinrix (which combines Hep A and Hep B), and Pediarix (which combines DTaP, Hep B, and IPV). This reduces the number of needed injections, which especially for children is very desirable, but also lowers the per shot cost of vaccination. These are usually the individual vaccines that could be given individually combined in a combination shot.

All existing and approved vaccines against SARS-COV-2 target only one protein complex of this virus: the S1 part of the spike structural protein, which contains the receptor binding domain (RBD) with which the virus docks to the ACE2 receptor of the host cell. The formation of antibodies against this protein complex is intended to block this attachment site and thus prevent the virion from entering the host cell.

Vaccine developments deviating from this focus on further points of attack in the virus such as the nucleocapsid protein. For example, in 2020, Herst et al. demonstrated a nine amino acid epitope located in the conserved region of the nucleocapsid protein, which is a target for vaccine development against Ebola virus. Immunity is achieved in the mouse model through epitope-mediated stimulation of cytotoxic T leukocytes (CTL), which is why the vaccine is called a CTL peptide vaccine. The authors suggest that a similar approach may be possible for the development of SARS-COV-2 immunity.

Slathia et al. (2020) used immunoinformatic methods to predict T- and B-cell epitopes present in the proteins of SARS-COV-2, where it is stated that the T-cell epitopes could be used in vaccine design and the B-cell epitopes could be used in the development diagnostic kits and vaccine design. They reported a population coverage analysis of the distribution of HLA alleles among different population groups with the goal to provide immunity to populations worldwide.

Ahmed et al. (2020) identified B-cell and T-cell epitopes of SARS-COV that are identical between SARS-COV and SARS-COV-2 using experimentally determined B-cell and T-cell epitopes with bioinformatics methods, providing a potential basis for therapy development.

A study by Chen et al. (2020) identified potential peptide epitopes for vaccine development. Here, surface accessibility, antigenicity, and other features were characterized for immunogen potential assessment. Potential B-cell epitopes on the spike protein and T-cell epitopes within the nucleocapsid protein were identified that could potentially elicit a CD4+ as well as a CD8+-dependent immune response and could be used for vaccine development.

In order to effectively repel pathogens, a vaccine is needed that simultaneously prevents the virion from entering the host cell and also prepares the immune system for intracellular defense mechanisms. However, during vaccine development, infection-enhancing antibodies (antibody-dependent enhancement, ADE) may occur that bind to the surface of viruses but do not neutralize them. This leads to enhanced uptake of the virus into a host cell and thus favors the spread and multiplication of the viruses within the host. The result is a more difficult course of disease in the event of a secondary infection.

Current coronavirus vaccines, especially those for SARS-COV-2, focus on rapid development of immunization platforms and do not consider the risk of ADE. For example, this problem was already encountered in vaccine development against the coronaviruses FIPV2,3, MERS4,5 and SARS, which is why no vaccines against these viruses have been developed and licensed to date. For SARS-COV-1, for example, an aggravated disease course was demonstrated after immunization of macaques, with non-neutralizing antibodies involving Fc receptors (FcRs) leading to viral infection of monocytes, macrophages and B cells.

In vaccine development, the problem of ADE can be overcome by a combined and balanced activation of T and B cells leading to a specific immune response throughout the viral life cycle. Thus, high technologies in bioinformatics, mathematical modeling, and artificial intelligence (AI) can predict different peptide epitopes that enable specific antibody properties or lead to balanced T-cell activation.

### Objection of the Invention/Technical Problem

Based on the state of the art, existing pharmaceutics and/or preparations for immunization against viral infectious diseases are faced with the problem that their subject range is not broad enough (one fits all approach) therefore, an objection of the present invention is to provide a pharmaceutical active combination preparation that reaches as broad a subject group as possible and at the same time is very specific response in the individual subject by means discussed further below in detail.

Based on the state of the art, single HLA antigen-based pharmaceutics face the problem that not every HLA allele that should/could be expressed according to the genotype is exposed on the cells, thus an objection of the present invention is to provide a pharmaceutical active combination which can target different HLA alleles simultaneously by means disclosed further below in detail.

In addition, known HLA antigen-based pharmaceuticals against viral infectious diseases face the problem that HLA antigen peptides are not stable in vivo over extended periods of time. Therefore, an objective of the present invention is to provide a pharmaceutically active combination preparation comprising at least one HLA antigen peptide, as disclosed in detail below, that is stable over an extended period of time in the subject and means for administering the preparation.

In the prior art, current immunizing agents struggle to combat the ever-evolving and mutating viral strains of viral infectious diseases, such as very recently coronaviruses, especially SARS-CoV-2, which vaccines with only one point of attack can more easily overcome, and therefore it is a concern of the present invention to provide a pharmaceutical composition for a combination preparation that targets at least two points of attack.

In some embodiments of the invention, it may provide pharmacologically active agents, as well as pharmaceutical compositions comprising such active agents, which can be used for the prophylaxis and/or treatment of viral infectious diseases; and to provide methods for the prevention and/or treatment of such viral infectious diseases, which include the administration and/or use of such active agents and compositions.

A severe shortcoming of known combination immunization strategies is that individually usable vaccines are combined in a combination vaccination, without modifying the vaccines for use in this application. In some embodiments of the invention, it may provide synthetic tandem and/or combination and/or overlapping antigen polypeptides matching epitopes from different viral infectious diseases, again matching different HLA A and HLA B antigen peptides and with a broad HLA allele coverage in order to immunize a broad portion of the worldwide population a single combination preparation against multiple viral infectious diseases.

### Solution

To overcome the technical problems of the prior art, a preferred embodiment of the present invention comprises of pharmacologically active agents (so called “antigen polypeptide” or “antigen polypeptide sequences”), pharmaceutical compositions and/or methods which have advantages over the active agents, compositions and/or methods currently in use and/or known in the prior art. These advantages result from the following further description.

A more preferred embodiment of the present invention is to provide therapeutically active synthetic polypeptides matching HLA antigen peptides that may be used as pharmacologically active synthetic polypeptides matching HLA antigen peptides and pharmacologically active agents, respectively, and to provide pharmaceutical compositions containing same, for the prevention and/or treatment of viral infectious disease and to provide methods for the prevention and/or therapeutic treatment of such infections and disorders involving the administration and/or use of such therapeutically active polypeptides matching HLA antigen peptides and compositions.

In a particular preferred embodiment of the invention, such synthetic polypeptides matching HLA antigen peptides are provided which are especially suitable for prophylactic and/or therapeutic use in warm-blooded animals, more particularly in mammals and most particularly in humans.

According to the invention, these objections are solved by a pharmaceutical composition for use in the therapeutic and/or prophylactic treatment of at least one viral infection, comprising a pharmacologically effective amount of at least one, preferably at least 2, more preferably 3, such as at least 4, at least 5, at least 6, at least 7, in particular at least 8, more particularly at least 10 and most particularly up to 12 antigen polypeptides comprising, inter alia, HLA antigen peptides, characterized in that exclusive targets are chosen, which correspond to viral epitopes from at least one viral infectious disease and are preferably chosen from different regions of the virus transcriptome, which are preferably expressed in the viral proteome.

According to a particular preferred embodiment of the invention, the these objections are solved by a pharmaceutical composition for use in the therapeutic and/or prophylactic treatment of coronavirus infections, comprising a pharmacologically effective amount of at least one, preferably at least 2, more preferably 3, such as at least 4, at least 5, at least 6, at least 7, in particular at least 8, more particularly at least 10 and most particularly up to 12 antigen polypeptides comprising, inter alia, HLA antigen peptides, characterized in that exclusive targets are chosen, such as preferably a coronavirus, more preferably a betacoronavirus, most preferably a sarbecovirus, such as a SARS-COV-2 spike glycoprotein (also called spike protein or S protein), nucleocapsid phosphoprotein (also called nucleocapsid protein or N protein), envelope protein (also called E protein), surface glycoprotein and ORF1ab polyprotein, wherein said antigen polypeptides particularly comprise or consist of sequences represented in the sequences SEQ ID NO: 38-68, more preferably 39-58 (as defined in more detail below).

Furthermore, these objections are solved by a pharmaceutical composition for use as a medicament, in particular for use in the therapeutic or prophylactic treatment of a viral infectious disease, in particular in a subject or group of subjects suffering from or at risk of suffering from a viral infectious disease, comprising of a pharmacologically effective amount consisting of at least 1, preferably at least 2, more preferably 3, such as at least 4, at least 5, at least 6, at least 7, in particular at least 8, more particularly at least 10 and most particularly up to 12 antigen polypeptide sequence(s) comprising at least one HLA-A and/or HLA-B antigen peptide, wherein the HLA antigen peptide corresponds, preferably is identical to at least one amino acid sequence in preferably at least one virus, more preferably at least two viruses, particularly preferably at least three viruses, most preferably at least four viruses while the amino acid sequence(s) are preferably expressed on the virus surface proteins, characterized in that the antigen polypeptide sequence in accordance with the present invention comprises or consists of the following scaffold amino acid sequence:
(a) a long peptide, i.e., a compound, construct, or polypeptide, wherein the amino acid sequence of which comprises one “HLA-A antigen peptide” or “HLA-B antigen peptide” as defined herein, preferably one native “HLA-A antigen peptide” or “HLA-B antigen peptide”, wherein the amino acid sequence comprises, in addition to the “HLA-A antigen peptide” or “HLA-B antigen peptide”, up to (but not more than) 1 to 30, more preferably 1 to 20, most preferably 1 to 15 amino acids; and/or
(b) a similarity peptide, i.e., a compound, construct, or polypeptide, wherein the amino acid sequence of which is as defined in item (a), wherein the “HLA-A antigen peptide” or “HLA-B antigen peptide” as defined herein having less than 100% sequence identity or similarity to the native HLA antigen peptide, such as having at least 85%, more preferably at least 90%, sequence identity; and/or
(c) a substitution peptide, i.e., a compound, construct, or polypeptide, wherein the amino acid sequence of which is as defined in item (a), wherein the “HLA-A antigen peptide” or “HLA-B antigen peptide” as defined herein having an amino acid sequence comprising or consisting essentially of only one amino acid substitution relative to the amino acid sequence of the native HLA antigen peptide; and/or
(d) a tandem peptide, i.e. a compound, construct, or polypeptide, wherein the amino acid sequence of which is as defined in item (a), comprising or consisting of at least 2, more preferably 3, such as at least 4 identical or different HLA-A and/or HLA-B antigen peptide sequences, both as defined in items (a) to (c), in which the HLA antigen peptides are optionally connected to each other by suitable linkers, more preferably by a suitable amino acid linker (so-called oligopeptides); and/or
(e) an overlapping tandem peptide, i.e. a compound, construct, or polypeptide, wherein the amino acid sequence of which is as defined in item (a), comprising or consisting of at least 2, more preferably 3, such as at least 4 identical or different HLA-A and/or HLA-B antigen peptide sequences, both as defined in items (a) to (c), in which the at least two HLA antigen peptides overlap in their amino acid sequence, and/or
(f) a tandem and/or overlapping tandem peptide, i.e. a compound, construct, or polypeptide, wherein the amino acid sequence of which is as defined in item (a), comprising or consisting of at least 2 identical or different HLA-A and/or HLA-B antigen peptide sequences, both as defined in items (a) to (c), in which the at least two HLA antigen peptides overlap in their amino acid sequence where the amino acid sequences match viral epitopes from at least two, preferably of at least three, alternatively four different viral infectious diseases.

The objection of the invention is particularly preferably solved by a pharmaceutical composition for use as a medicament, in particular for use in the therapeutic or prophylactic treatment of a coronavirus infectious disease, comprising a pharmacologically effective amount consisting of at least 2 and up to 12 antigen polypeptide sequence(s), each comprising at least one HLA-A and/or HLA-B antigen peptide, wherein the HLA antigen peptide corresponds to at least one amino acid sequence in a virus, wherein at least one of the antigen polypeptide sequence(s) comprises the following scaffold amino acid sequence: an overlapping tandem peptide, i.e. a compound, construct, or polypeptide, comprising or consisting of at least 2 identical or different HLA-A and/or HLA-B antigen peptide sequences, wherein at least one of the amino acid sequences comprises, in addition to the HLA-A antigen peptide or HLA-B antigen peptide, up to 1 to 30 amino acids; and/or wherein at least one of the amino acid sequences comprises an HLA-A antigen peptide or HLA-B antigen peptide with at least 90% sequence identity similarity to the native HLA-A or HLA-B antigen peptide, and/or wherein at least one of the amino acid sequences comprises an HLA-A antigen peptide or HLA-B antigen peptide as defined herein having an amino acid sequence comprising or consisting essentially of only one amino acid substitution relative to the amino acid sequence of the native HLA antigen peptide, in which at least two HLA antigen peptides overlap in their amino acid sequence and where the amino acid sequences match viral epitopes from at least two, preferably of at least three, alternatively four different viral infectious diseases.

The combination of different peptide epitopes, in accordance with the present invention, enables comprehensive targeting of a virus throughout the infection chain by allowing multiple points of attack. Thus, a virus can be repelled, its entry into the host cell prevented by antibodies, and its replication in the host cell prevented by activated Th1 defense cells. This is made possible by the combination of HLA antigens targeting different points of attack. Furthermore, in a particularly preferred embodiment of the current invention, the antigen polypeptides in accordance with the current invention match viral epitopes from at least two different viral infectious diseases, more preferably at least three different viral infectious diseases, most preferably at least four different viral infectious diseases with the technical effect of parallel immunization against different diseases with one combination preparation consisting of the aforementioned antigen polypeptides.

In an alternative embodiment of the current invention, the HLA antigen peptides used in the combination preparation are coronavirus exclusive, in particular wherein the HLA antigen peptides comprise sequences represented in the sequences SEQ ID NO: 1-37, 59-79, more preferably 2-37, 59-79 (as defined in more detail below).

Two or more HLA antigen peptides can also be present in an antigen polypeptide sequence in the form of a tandem peptide. Two or more HLA antigen peptides can also be present in the form of an overlapping tandem peptide.

Some examples (but not limited to) for particularly preferred antigen polypeptides, which are especially related to coronavirus infectious diseases, are listed in the sequences SEQ ID NOs.: 38-58 and 80-90, more preferably 39-58 and 80-90.

Further advantageous embodiments are given in the dependent claims.

### DESCRIPTION

### Advantages and Description of the Pharmaceutical Combination

In one embodiment, the pharmaceutical use comprises inducing an immune response against viral infectious disease in a subject. In one embodiment, the pharmaceutical use comprises a therapeutic or prophylactic treatment of a viral infectious disease. In one embodiment, the pharmaceutical composition or medical preparation described herein is for administration to a human.

In one embodiment, the virus is an RNA virus. In one embodiment, the viral infectious disease is a single stranded RNA virus. In one embodiment, the virus is a negative-strand RNA virus (−ssRNA virus). In one embodiment, the virus is a positive-strand RNA virus (−ssRNA virus). In one embodiment, the virus is a negative-strand RNA virus (−ssRNA virus). In one embodiment, the virus is an DNA virus. In one embodiment, the virus is an reverse transcribing virus (retrovirus). In one embodiment, the viral infectious disease is a coronavirus. In one embodiment, the viral infectious disease is a betacoronavirus. In one embodiment, the viral infectious disease is sarbecovirus. In one embodiment, the viral infectious disease is SARS-CoV-2, especially a wild-type (WT) or mutant SARS-COV-2, such as the Alpha (B.1.1.7), or Beta (B.1.351), or Gamma (P. 1), or Delta (B.1.617.2) and/or Omicron variant (B.1.1.529).

The present invention relates to a pharmaceutical composition, in particular a combination agent, to effect prophylactic protection against infection with a viral infectious disease and to prevent severe disease progression in the event that an infection has occurred. In addition to B-cell-mediated antibody generation, the main focus of the current invention is on the T-cell response. Preferably, administration of the combination agent induces more than 80%, more preferably 85% and most preferably 90% neutralizing antibodies against viral infectious disease, with the proportion of non-neutralizing antibodies preferably being less than 10%, particularly preferably less than 5%, and most preferably less than 2%. Advantageously, antibody dependent enhancement (ADE) is thus reduced. As described herein, but without limitation to any explanation, mechanism of action or hypothesis in the present invention, up to 12 different targets of amino acid sequences of the invention have been determined based on their ability and interaction with specific virus structures. These structures of amino acid sequences of the invention are (as described below).

A preferred embodiment of the present invention relates to a pharmaceutical combination composition, in particular a combination agent, to effect prophylactic protection against infection with preferably at least two different viral infectious diseases, more preferably against three different viral infectious diseases and to prevent severe disease progression in the event that an infection has occurred. In addition to B-cell-mediated antibody generation, the main focus of the current invention is on the T-cell response. Preferably, administration of the combination agent induces more than 80%, more preferably 85% and most preferably 90% neutralizing antibodies against at least one, preferably against two, most preferably at least three of the viral infectious diseases the combination composition is intended to immunize against, with the proportion of non-neutralizing antibodies preferably being less than 10%, particularly preferably less than 5%, and most preferably less than 2%. Advantageously, antibody dependent enhancement (ADE) is thus reduced. As described herein, but without limitation to any explanation, mechanism of action or hypothesis in the present invention, preferably at least two, more preferably up to 12 different targets of amino acid sequences (also referred to as synthetic antigen polypeptides) of the invention have been determined based on their ability and interaction with specific virus structures. These structures of amino acid sequences of the invention are (as described below).

This has the advantage that the immunization against at least two different viral infectious diseases can be archived with only one preparation consisting of synthetic polypeptides matching different parts of the virus epitopes, therefore reducing the amounts of injections needed in total while also requiring less active ingredients than classic combination vaccinations (which are made up of multiple individual vaccines).

In one embodiment, HLA antigen peptides in accordance with the present invention are corresponding to MHC class I and/or II complexes.

In one embodiment, HLA antigen peptides in accordance with the present invention are corresponding to MHC class I and/or II complexes, particularly for the immunization and/or treatment of at least one viral infectious disease.

One aspect of the invention discloses HLA antigen peptides in accordance with the present invention are corresponding to MHC class I and/or II complexes, particularly for the immunization and/or treatment of coronaviruses, such as SARS-COV-2 (see particularly preferred examples in Tables 1-8).

In a particularly preferred embodiment of the invention, HLA antigen peptides in accordance with the present invention are corresponding to MHC class I and/or II complexes and are matching the epitope of a viral infectious disease. In a most preferred embodiment of the invention, the HLA antigen peptides in accordance with the present invention are corresponding to MHC class I and/or II complexes while matching the epitope of at least two viral infectious diseases, more preferably at least three viral infectious diseases, particularly preferably at least three

Advantageously, the use/application of the pharmaceutical composition according to the invention or the specific combination of antigen peptides contained therein corresponding to the MHC class I complexes with at least one identical HLA allele is not merely a passive immunization (as in the case of treatment with antibodies, e.g. Herceptin) but an active immunization (i.e. specific activation of the T cells or B helper cells via information carriers).

For binding to a T cell receptor, an HLA antigen peptide of the invention typically has in its amino acid sequence one or more amino acid residues or one or more segments of amino acid residues (i.e., with each “segment” comprising two or more amino acid residues located adjacent to or in close proximity to each other, viz. in the primary or tertiary structure of the amino acid sequence) through which the amino acid sequence of the invention can bind to a T cell receptor (particularly a binding pocket thereof), the amino acid residues or portions of the amino acid residues thus forming the “anchor” for binding to a T cell receptor (also referred to herein as “anchor amino acids”). Determination of this “anchor” can be determined, for example, by in silico methods (e.g., the artificial neural network NNAlign used in the publicly available NetMHC-4.0 server).

The HLA antigen peptides provided by the present invention are preferably present substantially as a mixture of at least one HLA antigen peptide of the invention having a sequence described herein (as defined herein) or form part of a protein or polypeptide which may comprise or consist essentially of one or more HLA antigen peptides of the invention, and which may optionally comprise one or more pharmaceutically active HLA antigen peptides (wherein the HLA antigen peptides in a so-called oligopeptide are all optionally connected via one or more linkers).

For example, and without limitation, the antigen peptides of the invention may be used as a binding moiety in such a protein or antigen polypeptide, which may optionally include one or more additional amino acid sequences that may serve as a binding moiety (i.e., against one or more targets other than a T cell receptor) to provide a monovalent, multivalent, or multispecific antigen polypeptide of the invention as described herein, respectively. Such a protein or antigen polypeptide may also be present in substantially as a mixture of at least one protein or polypeptide having at least one sequence described herein (as defined herein). It has been shown that a pharmaceutical composition comprising a specific combination of antigen polypeptides comprising one or more HLA antigen peptides corresponding to MHC class I complexes and MHC class II complexes disclosed herein is particularly advantageous in that it is capable of specifically activating T cells as well as specifically activating B cells.

Classical methods for the treatment of infectious diseases of the invention are, for example, the injection of vaccines (also: vaccines). Comprehensively, a biologically or genetically engineered antigen consists mostly of protein or DNA or RNA segments or killed or attenuated pathogens. A vaccine can consist of an antigen from a single pathogen, as well as a mixture of several antigens from different pathogens or strains of pathogens and possibly other additives (adjuvants). In the context of a vaccination, the vaccine serves to specifically activate the immune system with regard to a specific pathogen or group of pathogens. For this purpose, the reaction of the immune system is exploited, which provokes an immune response in the presence of pathogens or their specific parts (e.g. surface proteins as antigens). This leads to the formation of antibodies and specialized T-helper cells, which then provide a long-lasting protection against the respective pathogen, which, depending on the pathogen, can last between a few years and lifelong. Classic known vaccine types include, for example, conjugated vaccines, DNA vaccines, RNA vaccines, T-cell vaccination (TCV).

In one aspect of the current invention, the inventors disclose the significant achievement to have found that for effective treatment with the antigen polypeptides used in accordance with the invention, with different target sequences of the different peptides of SARS-COV-2, “Antibody Dependent Enhancement” (ADE) can be avoided in the individual to be treated. ADE has occurred in the development of vaccines against the coronaviruses FIPV2,3, MERS4,5, and SARS. The objective of overcoming the ADE problem led to the development of the vaccine of the present invention, which causes combined and balanced T- and B-cell activation, leading to multiple specific immune responses throughout the viral replication cycle. The development of high technologies in bioinformatics, mathematical modeling and artificial intelligence (AI) allows for the first time to specifically predict epitopes that elicit specific antibody properties or lead to balanced T- and B-cell activation.

It is also an outstanding achievement of the inventors to have discovered that a pharmaceutical composition based on antigen polypeptides is particularly effective when both T lymphocytes (T cells for short) and B lymphocytes (B cells for short) are activated by them. T and B cells belong to the lymphocyte cell group and play an important role in the human immune system. T cells recognize antigens via a specific receptor, the so-called T cell receptor (TCR). However, for this to occur, the antigen must be offered to the T cell by an antigen-presenting cell (APC) via a class I or class II HLA. Stable binding of the T cell to the APC requires the participation of so-called auxiliary proteins. These include CD4 and CD8 (CD=“Cluster of Differentiation”), with the presence of such a cluster denoted by a “+” (CD4+, CD8+).

T cell lymphocytes contain the CD4+ and the CD8+ positive T cells. T cells that carry the CD4 feature are also called CD4-positive T cells or T helper cells. In normal adult blood, CD4+ T cells account for 27-57% of lymphocytes, or approximately 310-1,570 cells/μl. The group of CD8-positive (CD8+) T cells is also called cytotoxic or killer T cells. They play a special role in killing the body's own cells that are infected by pathogens, such as viruses.

In contrast, B cells are the only cells capable of producing antibodies and, together with T cells, make up the critical component of the adaptive immune system. While T cells are involved in the cell-mediated immune response, B cells are the carriers of the humoral immune response (and are responsible for the formation of antibodies). It has been shown in experiments that pharmaceutical compositions containing an absolute concentration per antigen polypeptide of preferably more than 100 μg and particularly preferably more than 500 μg are quite preferred, as this greatly intensifies the information and thus the activation and/or training of the immune system. In one embodiment, the concentration of a peptide is 600 μg. Accordingly, 600 μg×12=7,200 μg are contained in the pharmaceutical composition (per vial). It is highly advantageous if the pharmaceutical composition comprises the adjuvants already mentioned above, which, when the composition is applied to a patient, are capable of intensifying the body's immune response.

Comprehensive of a balanced Th1/Th2 based robust immune response, CpG (oligonucleotide of cytidine and guanosine) is a Toll-like receptor 9 (TLR-9) agonist and is, for example, a component of the market-approved prophylactic HBV vaccine Heplisav-B (Dynavax, USA). The letter C stands for the nucleotide cytosine, the p for phosphate and the G for the nucleotide guanine. The targeted activation of TLR9 by CpG oligonucleotides opens up a wide range of possibilities for using these compounds, for example, to improve the efficacy of vaccines. In the context of prophylactic HBV vaccination, the use of CpG leads to a shortening of the vaccination schedule from three (all other approved HBV vaccines) to two vaccinations. In addition, CpG as an adjuvant in Heplisav-B also results in a better response in type 2 diabetics, smokers, the obese, and the elderly, who generally show poorer response rates.

Furthermore, it is an outstanding achievement of the inventors to have discovered a pharmaceutical composition of up to 12 amino acid sequences used in accordance with the present invention which can take advantage of highly preserved parts of a virus, thus enabling a vaccination more effective against new mutations.

Furthermore, in an aspect of the current invention, it is an outstanding achievement of the inventors to have discovered a pharmaceutical composition of up to 12 amino acid sequences used in accordance with the present invention which can take advantage of highly preserved parts of a selection of at least two viral infectious diseases by comparing them to different mutants, related viruses, wild-types and other related viruses, thus enabling a vaccination more effective against new mutations.

In one embodiment of the invention relating to coronaviruses, the spike glycoprotein consists of trimers that form large structures, referred to as spikes or peplomers, which project from the surface of the virion. The spike protein is divided into two parts, the S1 and S2 regions. The S1 subunit is responsible for the interaction with the cells receptor proteins and docks to the angiotensin converting enzyme 2 (ACE2) membrane protein. The S2 region consist of the fusion peptide, which function is to destabilize the host cell membrane and is mostly made up of hydrophobic amino acids. The S2 region is more conserved in coronaviruses compared to the S1 region. In a preferred embodiment of the invention, at least one amino acid sequence is selected to target the S2 region of the coronavirus specifically, since for effective entry of a virion into a host cell, both functions are required: a) docking to the ACE2 receptor via S1 and b) fusion of the host cell and virion membranes primed by the serine 2 transmembrane protease TMPRSS2 which is able to cleave S1/S2, therefore blocking the fusion protein S2 is equally effective as blocking the receptor docking mechanism (S1) as the virus requires booth S1 and S2 motives for effective virion penetration into the host cell.

In a alternative embodiment of the present invention at least one of the antigen polypeptide sequences of the pharmaceutical composition comprises epitopes of the SARS-COV-2 spike glycoprotein, in a more preferred embodiment, at least one of the antigen polypeptide sequences of the pharmaceutical composition comprises epitopes of the of the S2 membrane fusion subunit of the spike protein specifically rather than the S1 receptor binding domain (RBD). In this respect, blocking the S2 fusion protein by antibodies is expected to advantageously show the same efficacy as blocking the S1-mediated receptor docking mechanism. In contrast to the S1 subunit, the S2 subunit is highly conserved and shows only a low mutation rate. Advantageously, the use of S2 specific epitopes results in improved protection against different SARS-COV-2 strains.

It is a serious shortcoming of existing vaccines that only small parts of the epitope of a virus are targeted. In the case of coronavirus, for example, vaccines target only the spike protein, which is subject to strong mutations and therefore easily bypassed by new viral variants such as the alpha (B.1.1.7) or beta (B.1.351) or gamma (P. 1) or delta (B.1.617.2) and/or omicron (B.1.1.529) variants.

Therefore, it is an outstanding achievement of the inventors that in a preferred embodiment of the invention, at least two amino acid sequences are selected to target the virus in all reproductive steps, not just the spike glycoprotein, nucleocapsid phosphoprotein, envelope protein, surface glycoprotein, and ORF1ab polyprotein required for membrane fusion, wherein the peptides particularly comprise sequences shown in SEQ ID NOs: 38-58, more preferably 39-58.

Another serious shortcoming of existing combination vaccines is that they consist of single active ingredients against a specific virus or viral infectious disease, whereas antigen polypeptides according an aspect of the present invention may contain parts of different viral epitopes in the same amino acid sequence, and a combination of antigen polypeptides as a combination preparation may combine different viral epitopes to provide a multilayered and more robust immunization against multiple viral infectious diseases.

It is a further outstanding achievement of the inventors to have discovered that a combination preparation containing at least two antigen polypeptides, preferably selected from SEQ ID NOs: 38-58, more preferably 39-58, preferably match a total of at least 20, more preferably at least 25, in a particular embodiment at least 30 HLA alleles, advantageously allowing more than 95% of the world's population to be immunized with the pharmaceutical composition of the invention. This allows a worldwide use of the vaccination without need for adaptation for different populations. In an alternative embodiment, if the combination preparation in accordance with the present invention contains the 12 antigen polypeptide sequences SEQ ID NOs.: 38-49, a total of 37 HLA alleles are matched, allowing more than 95% of the world population to be immunized.

In a preferred embodiment of the invention, where at least two amino acid sequences, preferably at least two HLA antigen polypeptides are selected for the pharmaceutical composition in accordance with the present invention, these two sequences are preferably selected to target at least two different parts of a viral epitope. In a particularly preferred embodiment, at least three sequences are selected to match at least three different regions, in a most preferred embodiment at least four regions corresponding to different parts of the virus are chosen. The benefit of this embodiment is to target different steps in reproductive cycle of the virus, thus reducing the possibility of mutation caused resistance.

In an alternate embodiment of the invention, where at least two amino acid sequences, preferably at least two HLA antigen peptides are selected for the pharmaceutical composition in accordance with the present invention, these two sequences are preferably selected to target at least two different parts of the viral epitope, chosen for example from the spike glycoprotein, nucleocapsid phosphoprotein, envelope protein, surface glycoprotein and ORF1 ab polyprotein of the SARS-COV-2 virus. In a particularly preferred embodiment, at least three sequences are selected to match at least three different regions, in a most preferred embodiment at least four regions corresponding to different parts of the virus are chosen. The benefit of this embodiment is to target different steps in reproductive cycle of the virus, thus reducing the possibility of mutation caused resistance.

In a preferred aspect of the current invention the pharmaceutical composition contains at least two antigen polypeptides which are selected to match viral epitopes of the at least one viral infectious disease in such manner to match enough HLA alleles to archive prophylactic protection for at least 95% of the world population and to match different viral proteins, which are highly conserved and thus be more resistant against mutations.

In a preferred aspect of the current invention the pharmaceutical composition contains at least two antigen polypeptides which are selected to match viral epitopes of the coronavirus, particularly SARS-COV-2, particularly preferably selected from SEQ ID NOs: 38-58, most preferably 39-58 in such manner to match enough HLA alleles to archive prophylactic protection for at least 95% of the world population and to match different viral proteins, which are highly conserved and thus be more resistant against mutations.

Bis-(3′,5′)-cyclic-adenosine monophosphate is a bacterial messenger involved in the control of cell wall metabolism, osmotic stress responses and sporulation. C-di-AMP is a STING agonist that also induces a balanced Th1/Th2 immune response. STING (STimulator of INterferon Genes) is a protein expressed by various cell types (including T cells, macrophages, and dendritic cells) that acts as a sensor of cytosolic, pathogen DNA and induces type I interferon production as part of the innate immune response.

Aluminum salts (usually a mixture of aluminum sulfate, potassium or sodium hydroxide, and phosphate) have been used as adjuvants for more than 100 years and are adjuvant components of a variety of market-approved vaccines. Aluminum-containing adjuvants adsorb antigens most commonly due to electrostatic interactions. The exact mechanism of how the immune response is stimulated is still the subject of research. A depot mechanism is assumed, that the adjuvant gradually releases the adsorbed antigen at the site of injection. This mechanism is supported by the observation that antigens that bind more strongly to the adjuvant cause a stronger immune response. In particular, alum-dependent T cell differentiation to CD4+ Th2 cells is an important effect in prophylactic vaccines for targeting an antibody-based immune response. Alum can further promote antigen uptake in antigen-presenting cells (APCs) and enhance MHC-II gene cluster expression.

The lipopeptide Pam3Cys-GDPKHPKSF is a TLR-2/TLR-1 ligand with the precise designation N-palmitoyl-S-[2,3-bis(palmitoyloxy)-(2R)-propyl]-(R)-cysteinyl-GDPKHPKSF. This water-soluble adjuvant induces potent responses from CD8 and CD4 positive T cells.

MF59 and AS03 are squalene-based oil in water emulsion adjuvants which were successfully used in a variety of market approved human vaccines. They can lead to an improved and more balanced immunity, possibly by stimulating the CD4+ and CD8+ T-cell immune responses. MF59 is currently used as an adjuvant in the United States Canada and Europe.

Poly-IC is a TLR3/MDA5 agonist. Poly I:C binds to and activates TLR3 in animals, subsequently activating the innate immune response. TLR3 is produced by B cells, macrophages and dendritic cells. Poly I:C has been studied as an adjuvant in vaccines and for the induction of apoptosis in cancer cells.

The physiological pH-value of the blood of a healthy adult human is between 7,35-7,45, thus for better physiological compatibility the pH of the pharmaceutical composition is considered, the total charge of the amino acids in the pharmaceutical composition should be mostly basic or only slightly acidic. In a preferred embodiment of the present invention single peptides have a maximum excess of acidic amino acids of 5, more preferred of 4, and most preferred of 3. The preferred excess of basic amino acids is up to 10 amino acids, more preferred is an excess of 8, and most preferred is an excess of 6 basic amino acids. Amino acid excess is hereby defined as absolute value of the difference of basic and acid amino acids of the sequence.

In one aspect of the invention related to a coronavirus, the S2 region of the corona spike protein contains the fusion peptide, a stretch of mainly hydrophobic amino acids, thus in a preferred embodiment of the invention, the amino acid sequence (as set forth in the Examples SEQ ID NOs: 38-58, more preferably 39-58) is mainly compromised of hydrophobic amino acids, preferably the share of hydrophobic amino acids is between 20-90% of the sequence, more preferably between 35%-80% of the sequence, while having a preferred share of hydrophilic amino acids between 0%-60% of the total amino acids of the sequence bearing hydrophobic amino groups, more preferred a share of hydrophilic amino acids between 10%-50% of the total amino acids of the sequence.

In a preferred embodiment of the invention, the antigen polypeptides used in the pharmaceutical composition have an amino acid length between 9 and 40, more preferred between 9 and 35 and most preferred between 9 and 30 amino acids. In an especially preferable embodiment of the invention, the length of the antigen polypeptides is between 9 and 20, most preferably between 9 and 15 amino acids in length. In an alternative embodiment of the invention the amino acid chain is linear (not cyclic).

In an alternative embodiment of the invention, the antigen polypeptides can be further modified at the C or N-terminus. In a preferred embodiment the modification of the C-terminus can reduce excess positive charge to align to the physiological pH-value of the subjects, while the modification of the N terminus can reduce the basic character of the peptide. The means to modify peptides in this way are known to the person skilled in the art, for example using the Merrifield peptide synthesis.

In one embodiment, an “antigen polypeptide”, preferably for use as a medicament, in particular for use in the therapeutic or prophylactic treatment of a coronavirus infectious disease, in particular in a subject or group of subjects suffering from or at risk of suffering from a viral infectious disease, is preferred corresponding to at least one HLA antigen peptide corresponding to MHC class I comprising the following scaffold amino acid sequence:
(a) a long peptide, i.e., a compound, construct, or polypeptide, wherein the amino acid sequence of which comprises one “HLA-A antigen peptide” or “HLA-B antigen peptide” as defined herein, preferably one native “HLA-A antigen peptide” or “HLA-B antigen peptide”, most preferably one “HLA-A antigen peptide” or “HLA-B antigen peptide” selected from the group consisting of SEQ ID NOs: 1-37 and 59-79, wherein when the amino acid sequence comprises an aforementioned HLA antigen peptide, the amino acid sequence comprises, in addition to the “HLA-A antigen peptide” or “HLA-B antigen peptide”, up to (but not more than) 1 to 30, more preferably 1 to 20, most preferably 1 to 15 amino acids; and/or
(b) a similarity peptide, i.e., a compound, construct, or polypeptide, wherein the amino acid sequence of which is as defined in item (a), wherein the “HLA-A antigen peptide” or “HLA-B antigen peptide” as defined in item (a) having less than 100% sequence identity or similarity to the native HLA antigen peptide, such as having at least 85%, more preferably at least 90%, sequence identity; and/or
(c) a substitution peptide, i.e., a compound, construct, or polypeptide, wherein the amino acid sequence of which is as defined in item (a), wherein the “HLA-A antigen peptide” or “HLA-B antigen peptide” as defined in item (a) having an amino acid sequence comprising or consisting essentially of only one amino acid substitution relative to the amino acid sequence of the native HLA antigen peptide; and/or
(d) a tandem peptide, i.e. a compound, construct, or polypeptide, wherein the amino acid sequence of which is as defined in item (a), comprising or consisting of at least 2, more preferably 3, such as at least 4 identical or different HLA-A and/or HLA-B antigen peptide sequences, both as defined in items (a) to (c), in which the HLA antigen peptides are optionally connected to each other by suitable linkers, more preferably by a suitable amino acid linker (so-called oligopeptides); and/or
(e) an overlapping tandem peptide, i.e. a compound, construct, or polypeptide, wherein the amino acid sequence of which is as defined in item (a), comprising or consisting of at least 2, more preferably 3, such as at least 4 identical or different HLA-A and/or HLA-B antigen peptide sequences, both as defined in items (a) to (c), in which the at least two HLA antigen peptides overlap in their amino acid sequence; and/or
(f) a tandem and/or overlapping tandem peptide, i.e. a compound, construct, or polypeptide, wherein the amino acid sequence of which is as defined in item (a), comprising or consisting of at least 2 identical or different HLA-A and/or HLA-B antigen peptide sequences, both as defined in items (a) to (c), in which the at least two HLA antigen peptides overlap in their amino acid sequence where the amino acid sequences match viral epitopes from at least two, preferably of at least three, alternatively four different viral infectious diseases.

According to one preferred embodiment, one of the aforementioned amino acid sequences of the antigen polypeptide, in particular as defined in items (a) to (e), may consist in total of up to (but not more than) 10 to 40, more preferably up to 10 to 30, most preferably up to 10 to 25 amino acids, which has the advantage that the stability of the incorporated HLA-A antigen peptides and/or HLA-B antigen peptides is increased thereby. In particular, the addition of amino acids at the C- and/or N-terminus increases the stability (depending on the method of administration e.g., in the bloodstream, in muscle tissue, in skin tissue) after administration to a subject, as they may be undergoing partial degradation from the C- and/or N-terminus after administration.

In an especially preferred embodiment of the current invention a pharmaceutical composition for use as a medicament is provided, in particular for use in the therapeutic or prophylactic treatment of a coronavirus infectious disease. The composition comprises a pharmacologically effective amount consisting of at least 2 and up to 12 antigen polypeptide sequence(s), each comprising at least one HLA-A and/or HLA-B antigen peptide, wherein the HLA antigen peptide corresponds to at least one amino acid sequence in a virus, wherein at least one of the antigen polypeptide sequence(s) comprises the following scaffold amino acid sequence: an overlapping tandem peptide, i.e. a compound, construct, or polypeptide, comprising or consisting of at least 2 identical or different HLA-A and/or HLA-B antigen peptide sequences, wherein at least one of the amino acid sequences comprises, in addition to the HLA-A antigen peptide or HLA-B antigen peptide, up to 1 to 30 amino acids; and/or wherein at least one of the amino acid sequences comprises an HLA-A antigen peptide or HLA-B antigen peptide with at least 90% sequence identity similarity to the native HLA-A or HLA-B antigen peptide, and/or wherein at least one of the amino acid sequences comprises an HLA-A antigen peptide or HLA-B antigen peptide as defined herein having an amino acid sequence comprising or consisting essentially of only one amino acid substitution relative to the amino acid sequence of the native HLA antigen peptide, in which at least two HLA antigen peptides overlap in their amino acid sequence and where the amino acid sequences match viral epitopes from at least two, preferably of at least three, alternatively four different viral infectious diseases.

This has the technical advantage of allowing for a broader immunization with less peptides then previously, simultaneously allowing for immunization against multiple viral infectious diseases.

In a preferred embodiment, the pharmaceutical comprises a pharmacologically effective amount consisting of at least 2 and up to 12 antigen polypeptides.

In a preferred embodiment, the pharmaceutical consist of a pharmacologically effective amount consisting of at least 2 and up to 12 antigen polypeptides.

In a preferred embodiment, the pharmaceutical comprises a pharmacologically effective amount comprises at least 2 and up to 12 antigen polypeptides.

In a preferred embodiment, the pharmaceutical consist of a pharmacologically effective amount comprising of at least 2 and up to 12 antigen polypeptides.

In a preferred embodiment, the C-terminus and/or N-terminus of the antigen polypeptide comprises so called “capping groups”. A preferred capping group at the N-terminus can be an acetyl group. A preferred capping group at the C-terminus can be an amide group. These capping groups not only increase metabolic stability but may also increase in vitro binding affinities.

According to one preferred embodiment, one of the aforementioned amino acid sequences of an antigen polypeptide, in particular as defined in items (a) to (e), that may consist of up to (but not more than) 10 to 40, more preferably up to 10 to 30, most preferably up to 10 to 25 amino acids, additionally comprises so called “anchor amino acids” or “anchor residues” that strengthen the linkage to an HLA allele. For examples the antigen polypeptide as defined herein comprises one or more HLA-A antigen peptides and/or HLA-B antigen peptides flanked by an anchor amino acid in C-terminal and/or N-terminal position. Examples for anchor amino acids are acidic amino acids like D and/or E at the C-terminus and/or N-Terminus of the long HLA antigen peptide and/or hydrophobic amino acids like A, F, G, I, L, M, P, V and/or W in the center of the long HLA antigen peptide. Furthermore, those anchor residues include phosphorylated peptides, which can be recognized by T cells.

According to one preferred embodiment, the pharmaceutical composition comprises at least one tandem antigen polypeptide as defined in item (d) and/or one overlapping tandem antigen polypeptide as defined in item (e) that comprises at least two different HLA antigen peptides, such as (i) one HLA-A antigen peptide and at least one HLA-B antigen peptide or (ii) at least two HLA-A antigen peptide or (iii) at least two HLA-B antigen peptides. This has the advantage that multiple of targeting multiple HLA alleles. Thus, the inventors have found that although certain genotypes encode specific HLA alleles, these are not expressed and/or presented on the cells and are therefore not accessible. Furthermore, this allows different genotypes to be addressed by the administration of a single “antigen polypeptide” (as defined herein).

In addition, a tandem antigen polypeptide as defined in item (d) or an overlapping tandem antigen polypeptide as defined in item (e) can be tailored in such a way that one antigen polypeptide comprises at least two different HLA-A and/or HLA-B antigen peptides, wherein
(i) each one of the HLA-A and/or HLA-B antigen peptide located within the tandem antigen polypeptide or the overlapping tandem antigen polypeptide corresponds to an amino acid sequence located within a region of at least one virus related to a viral infectious disease, wherein the amino acid sequence of the first HLA-A and/or HLA-B antigen peptide differs from the amino acid sequence of the second or the other HLA-A and/or HLA-B antigen peptide(s) of the antigen polypeptide, for example corresponds to and thus “target” a different region within a specific virus sub-species. As a result, the tandem antigen polypeptide or the overlapping tandem antigen polypeptide according to the invention can be used to “target” a specific/single region of a virus, especially a specific/single region of a virus sub-species (such as only the spike S1 domain, S2 domain, nucleocapsid protein, envelope protein or ORF1ab polyprotein), but moreover for the use in broad targeting, preferably for use in the “targeting” of multiple regions. and/or
(ii) each one of the HLA-A and/or HLA-B antigen peptide located within the tandem antigen polypeptide or the overlapping tandem antigen polypeptide corresponds to an amino acid sequence located within a region of a different virus related to a viral infectious disease, wherein the amino acid sequence of the first HLA-A and/or HLA-B antigen peptide differs from the amino acid sequence of the second or the other HLA-A and/or HLA-B antigen peptide(s) of the antigen polypeptide in this way that first HLA-A and/or HLA-B antigen peptide corresponds to and “target” a first region in a first virus, especially first virus sub-species and the second or the other HLA-A and/or HLA-B antigen peptide(s) corresponds to and “target” a region in a second virus, especially second virus sub-species. As a result, the tandem antigen polypeptide or the overlapping tandem antigen polypeptide according to the invention can be used not only for the treatment of a specific/single viral infectious disease, especially specific/single virus sub-species, but moreover for the use in the broad-spectrum immunization, preferably for use in the therapeutic or prophylactic treatment of a family-spanning, preferably at least subfamily-spanning, particularly preferably genus-spanning, especially subgenus-spanning viral infectious disease.
(iii) each one of the HLA-A and/or HLA-B antigen peptide located within the tandem antigen polypeptide or the overlapping tandem antigen polypeptide corresponds to an amino acid sequence located within a region of a different virus related to a viral infectious disease, wherein the amino acid sequence of the first HLA-A and/or HLA-B antigen peptide differs from the amino acid sequence of the second or the other HLA-A and/or HLA-B antigen peptide(s) of the antigen polypeptide in this way that first HLA-A and/or HLA-B antigen peptide corresponds to and “target” a first region in a first virus and the second or the other HLA-A and/or HLA-B antigen peptide(s) corresponds to and “target” a region in a second virus, especially if the second virus is from another viral infectious disease, such as preferably another viral realm, more preferably from another viral kingdom, especially from another viral phyla, such as another subphyla, more especially preferably from another viral class, alternatively from another viral order, preferably such as a viral suborder, most preferably from another family, such as a subfamily, most especially preferably from another viral genera, such as another subgenera, and most alternatively preferably from another species. As a result, the tandem antigen polypeptide, or the overlapping tandem antigen polypeptide according to the invention can be used not only for the treatment of a specific/single viral infectious disease, especially specific/single virus sub-species, but moreover for the use in the broad-spectrum immunization, preferably for use in the therapeutic or prophylactic treatment of at least two different, preferably not closely related viral infectious diseases.

In a preferred embodiment of the current invention, the antigen polypeptides of (iii) correspond to the epitopes of at least two different viral diseases, preferably from different species, alternatively from different families, especially preferably from different viral realms.

In a more preferred embodiment of the current invention, the antigen polypeptides of (iii) correspond to the epitopes of at preferably at least two, more preferably at least three, most preferably at least four different viral diseases, most especially preferably up to five viruses from different species, alternatively from different families, especially preferably from different viral realms.

In an alternative embodiment of the invention related to a coronavirus, a tandem antigen polypeptide as defined in item (d) or an overlapping tandem antigen polypeptide as defined in item (e) can be tailored in such a way that one antigen polypeptide comprises at least two different HLA-A and/or HLA-B antigen peptides, wherein
(i) each one of the HLA-A and/or HLA-B antigen peptide located within the tandem antigen polypeptide or the overlapping tandem antigen polypeptide corresponds to an amino acid sequence located within a region of a coronavirus, such as within the spike S1 domain, S2 domain, nucleocapsid protein, envelope protein and/or ORF1ab polyprotein, wherein the amino acid sequence of the first HLA-A and/or HLA-B antigen peptide differs from the amino acid sequence of the second or the other HLA-A and/or HLA-B antigen peptide(s) of the antigen polypeptide, for example corresponds to and thus “target” a different region within a specific virus sub-species (such SARS-COV 1, SARS-COV 2, MERS-COV). As a result, the tandem antigen polypeptide or the overlapping tandem antigen polypeptide according to the invention can be used to “target” a specific/single region of a coronavirus, especially a specific/single region of a virus sub-species (such as only the spike S1 domain, S2 domain, nucleocapsid protein, envelope protein or ORF1ab polyprotein), but moreover for the use in broad targeting, preferably for use in the “targeting” of multiple regions. and/or
(ii) each one of the HLA-A and/or HLA-B antigen peptide located within the tandem antigen polypeptide or the overlapping tandem antigen polypeptide corresponds to an amino acid sequence located within a region of a different coronavirus, such as within the spike S1 domain, S2 domain, nucleocapsid protein, envelope protein and/or ORF1ab polyprotein, wherein the amino acid sequence of the first HLA-A and/or HLA-B antigen peptide differs from the amino acid sequence of the second or the other HLA-A and/or HLA-B antigen peptide(s) of the antigen polypeptide in this way that first HLA-A and/or HLA-B antigen peptide corresponds to and “target” a first region in a first coronavirus, especially first virus sub-species (such SARS-CoV 1, SARS-COV 2, MERS-COV) and the second or the other HLA-A and/or HLA-B antigen peptide(s) corresponds to and “target” a region in a second coronavirus, especially second virus sub-species. As a result, the tandem antigen polypeptide or the overlapping tandem antigen polypeptide according to the invention can be used not only for the treatment of a specific/single coronavirus, especially specific/single virus sub-species (such as SARS-COV 1, SARS-COV 2, MERS-CoV), but moreover for the use in the broad-spectrum immunization, preferably for use in the therapeutic or prophylactic treatment of a family-spanning, preferably at least subfamily-spanning, particularly preferably genus-spanning, especially subgenus-spanning coronavirus infectious disease.

In one embodiment especially related to a coronavirus, the “antigen polypeptide” corresponding to at least one HLA antigen peptide corresponding to MHC class I comprises or consists of an amino acid sequence according to the following sequences:
(a) an amino acid sequence comprising or consisting of an amino acid sequence selected from the group consisting of SEQ ID NOs: 38-58, more preferably of SEQ ID NO: 43, 44, 50, 51, 53, 54 and/or 58, most preferably an amino acid sequence of SEQ ID NO: 43, 44, 50, 54 and/or 58, and/or
(b) an amino acid sequence having less than 100% sequence identity or similarity to the amino acid sequence according to item (a), such as at least 85%, more preferably at least 90%, sequence identity (as defined herein) to an amino acid sequence of SEQ ID NOs: 38-58, more preferably of SEQ ID NO: 43, 44, 50, 51, 53, 54 and/or 58, most preferably to an amino acid sequence of SEQ ID NO: 43, 44, 50, 54 and/or 58; and/or
(c) an amino acid sequence, wherein the amino acid sequence of which is as defined in item (a), comprising or consisting essentially of only one amino acid substitution relative to the amino acid sequence(s) selected from the group consisting of SEQ ID NOs: 38-58, more preferably of SEQ ID NO: 43, 44, 50, 51, 53, 54 and/or 58, most preferably an amino acid sequence of SEQ ID NO: 43, 44, 50, 54 and/or 58; and/or
(d) a tandem peptide, i.e., a compound, construct, or polypeptide comprising or consisting of at least two identical or different peptide sequences of at least one sequence as defined in items (a) to (c) above, in which the amino acid sequences are optionally linked to each other by suitable linkers (so-called oligopeptides); and/or
(e) an overlapping tandem peptide comprising or consisting of at least 2, more preferably 3, such as at least 4 identical or different HLA-A and/or HLA-B antigen peptide sequences (as defined herein), encoding for at least two, different HLA-A and/or HLA-B antigen peptides (as defined herein), wherein the at least two HLA antigen peptides overlap in their amino acid sequence.

In one preferred embodiment related to a coronavirus, the pharmaceutical composition comprises at least one amino acid sequence according to the following sequences:
(a) an amino acid sequence comprising or consisting of an amino acid sequence selected from the group consisting of SEQ ID NOs: 38-58, more preferably 39-58; and/or
(b) an amino acid sequence having less than 100% sequence identity or similarity to the amino acid sequence according to item (a), such as having at least 85%, more preferably at least 90%, sequence identity (as defined herein) to an amino acid sequence selected from the group consisting of SEQ ID NOs: 38-58, more preferably 39-58; and/or
(c) an amino acid sequence, wherein the amino acid sequence of which is as defined in item (a), comprising or consisting essentially of only one amino acid substitution relative to the amino acid sequence(s) selected from the group consisting of SEQ ID NOs: 38-58, more preferably 39-58; and/or
(d) a tandem peptide, i.e., a compound, construct, or polypeptide comprising or consisting of at least two identical or different peptide sequences of at least one sequence as defined in items (a) to (c) above, in which the amino acid sequences are optionally linked to each other by suitable linkers (so-called oligopeptides); and/or
(e) an overlapping tandem peptide comprising or consisting of at least 2, more preferably 3, such as at least 4 identical or different HLA-A and/or HLA-B antigen peptide sequences (as defined herein), encoding for at least two, different HLA-A and/or HLA-B antigen peptides (as defined herein), wherein the at least two HLA antigen peptides overlap in their amino acid sequence.

In a particularly preferred embodiment, an antigen polypeptide according to the current invention, wherein the antigen polypeptide is an overlapping tandem polypeptide preferably containing between 2 to 10, more preferably containing between 2 to 8, most preferably containing between 2 to 6 different amino acid sequences (HLA antigen peptides) matching a viral epitope corresponding to MHC Class I and/or MHC Class II complexes. This has the technical effect of allowing more antigen targets to be concentrated on a small antigen polypeptide, preferably smaller than 25 amino acids, more preferably smaller than 20 amino acids in length, which drastically reduces the cost of the synthetic antigen polypeptides. Another advantage is that more specific antigens can be transferred with a single injection at the same polypeptide weight than with a non-overlapping antigen peptide, allowing a lower dose or higher immunization effect per injection.

In a preferred embodiment, more than one polypeptide can overlap with a single polypeptide in an overlapping tandem polypeptide, for example, two or more single antigen peptides matching MHC Class I complexes corresponding to viral epitopes can overlap with another single antigen peptide, thus creating an overlapping antigen polypeptide in the spirit of the current invention.

In one aspect of the current invention related to a coronavirus, the pharmaceutical composition comprises
(a) an amino acid sequence of SEQ ID NO: 43, 44, 50, 51, 53, 54 and/or 58, more preferably an amino acid sequence of SEQ ID NO: 43, 44, 50, 54 and/or 58, or the amino acid sequence having at least 85%, more preferably at least 90%, sequence identity (as defined herein) to an amino acid sequence of SEQ ID NO: 43, 44, 50, 51, 53, 54 and/or 58, more preferably an amino acid sequence of SEQ ID NO: 43, 44, 50, 54 and/or 58; and/or
(b) an amino acid sequence having less than 100% sequence identity or similarity to the amino acid sequence according to item (a), such as at least 85%, more preferably at least 90%, sequence identity (as defined herein) to an amino acid sequence of SEQ ID NO: 43, 44, 50, 51, 53, 54 and/or 58, more preferably to an amino acid sequence of SEQ ID NO: 43, 44, 50, 54 and/or 58; and/or
(c) an amino acid sequence, wherein the amino acid sequence of which is as defined in item (a), comprising or consisting essentially of only one amino acid substitution relative to the amino acid sequence(s) selected from the group consisting of SEQ ID NO: 43, 44, 50, 51, 53, 54 and/or 58, more preferably an amino acid sequence of SEQ ID NO: 43, 44, 50, 54 and/or 58; and/or
(d) a tandem peptide, i.e., a compound, construct, or polypeptide comprising or consisting of at least two identical or different peptide sequences of at least one sequence as defined in items (a) to (c) above, in which the amino acid sequences are optionally linked to each other by suitable linkers (so-called oligopeptides); and/or
(e) an overlapping tandem peptide comprising or consisting of at least 2, more preferably 3, such as at least 4 identical or different HLA-A and/or HLA-B antigen peptide sequences (as defined herein), encoding for at least two, different HLA-A and/or HLA-B antigen peptides (as defined herein), wherein the at least two HLA antigen peptides overlap in their amino acid sequence.

A preferred embodiment of the current invention are HLA antigen peptides corresponding to MHC class I complexes, in particular for use in the treatment or prophylaxis of a virus infectious disease, for example but not limited to Orthomyxoviridae, a virus family containing Alphainfluenzavirus, Betainfluenzavirus, Gammainfluenzavirus, Deltainfluenzavirus, Isavirus, Thogotovirus, and Quaranjavirus (negative-sense RNA viruses) and/or poliovirus (from the family of Picornaviridae with single-stranded positive-sense RNA), and/or Rotavirus (from the family of Reoviridae with double stranded RNA viruses), and/or tick-borne encephalitis virus (TBEV, from the family of Flaviviruses with a positive-strand RNA), and/or human papillomavirus (HPV, from the Papillomaviridae family of DNA viruses), in a person or group of persons who has contracted or is at risk of contracting such a aforementioned viral infectious disease and/or for a pharmaceutical composition according to the invention consisting of antigen polypeptides.

A preferred aspect of the present invention are HLA antigen peptides corresponding to MHC class I complexes, in particular for use in the treatment or prophylaxis of a coronavirus infectious disease such as SARS-COV-2 in a person or group of persons who has contracted or is at risk of contracting a coronavirus such as SARS-COV-2 and/or for a pharmaceutical composition according to the invention, wherein the HLA-A antigen peptides or HLA-B antigen peptides are preferably selected from the group consisting of the amino acid sequences shown in Table 1-6 or which have at least one mutation, preferably an amino acid substitution, with respect to one of these amino acid sequences.

Particularly preferred herein is the use of the foregoing HLA antigen peptides and/or antigen polypeptides for use in a method of treating at least one viral infectious disease in a patient or group of patients having at least one identical HLA allele, the method comprising administering/applying a treatment regimen to a subject or group of subjects such that a pharmacologically effective amount comprises at least one of the foregoing HLA antigen peptides.

Another aspect of the current invention is the use of the foregoing HLA antigen peptides and/or antigen polypeptides for use in a method of treating SARS-COV-2 in a patient or group of patients having at least one identical HLA allele, the method comprising administering/applying a treatment regimen to a subject or group of subjects such that a pharmacologically effective amount comprises at least one of the foregoing HLA antigen peptides.

In one embodiment of the invention related to coronaviruses, a pharmaceutical composition as defined herein is for use in therapeutic or prophylactic treatment of a coronavirus infectious disease in a living subject, wherein the therapeutic or prophylactic treatment comprises administering an effective amount of the pharmaceutical composition to a subject and/or group of subjects.

In one alternative embodiment of the invention related to coronaviruses, the HLA-A and/or HLA-B antigen peptide(s) corresponding to MHC class I complexes of the at least one antigen polypeptides, preferably as used in the pharmaceutical composition, is/are an amino acid sequence selected from the group consisting of SEQ ID NO: 1-37, 59-79, more preferably 2-37, 59-79.

In one embodiment, the antigen polypeptide(s), preferably as used in the pharmaceutical composition, is/are an amino acid sequence selected from the group consisting of SEQ ID NOs 38-58, more preferably 39-58.

In one embodiment, the pharmaceutical composition further comprises at least one amino acid sequence comprising or consisting of an HLA-A or HLA-B antigen peptide, wherein the HLA peptide corresponds, preferably is identical to at least one amino acid sequence in a virus causing a viral infectious disease, preferably from the transcriptome encoding the proteins of the virus presented on the surface of the virus, more preferably being part of the viral epitope. Preferably, the HLA-A or HLA-B antigen peptide comprises or consisting of an amino acid sequence as defined herein, more preferably, the amino acid sequence is selected from the group consisting of SEQ ID NO: 1-37, 59-79, more preferably 2-37, 59-79.

An “HLA-A antigen peptide corresponding to MHC class I complexes” is defined herein as an “HLA-A antigen peptide of the invention” (also called “HLA-A antigen peptide”) or “amino acid sequence of the invention” (as defined herein) comprising:
(a) an amino acid sequence preferably consisting of 6 to 12 amino acids, more preferably consisting of 7 to 10 amino acids, and most preferably consisting of 8 or 9 amino acids corresponding to MHC class I complexes which is similar or more preferably identical to a protein encoding RNA sequence in the genome of a viral infectious disease.
(b) preferably, the HLA-A antigen peptide is present in the pharmaceutical composition according to the invention at a concentration, as defined above, of at least 100 μg and particularly preferably at least 500 μg, alternatively preferably at an absolute concentration of at least 600 μg relative to the volume of the pharmaceutical composition to be applied.

Preferably, the HLA-A antigen peptides are selected as defined above under point (b).

According to a particularly preferred embodiment, an “HLA-A antigen peptide”, is preferred, comprising the following scaffold sequence:
(a) an amino acid sequence comprising or consisting of an amino acid sequence selected from the group consisting of SEQ ID NOs: 1, 3-7, 9, 16, 18-19, 21-23, 27, 29-31, 33-34, 36, wherein when the amino acid sequence comprises an aforementioned amino acid sequence, the amino acid sequence preferably further comprises up to (but not more than) 1 to 30, more preferably 1 to 20, most preferably 1 to 15 amino acids; and/or
(b) an antigen peptide comprising or consisting of an amino acid sequence having less than 100% sequence identity or similarity to the native HLA-A antigen peptide, such as having at least 85%, more preferably at least 90%, sequence identity (as defined herein) to an amino acid sequence selected from the group consisting of SEQ ID NOs: 1, 3-7, 9, 16, 18-19, 21-23, 27, 29-31, 33-34, 36, wherein when the amino acid sequence comprises an aforementioned amino acid sequence, the amino acid sequence preferably further comprises up to (but not more than) 1 to 30, more preferably 1 to 20, most preferably 1 to 15 amino acids; and/or
(c) an antigen peptide having an amino acid sequence comprising or consisting essentially of only one amino acid substitution relative to the amino acid sequence(s) selected from the group consisting of SEQ ID NOs: 1, 3-7, 9, 16, 18-19, 21-23, 27, 29-31, 33-34, 36, wherein when the amino acid sequence comprises an aforementioned amino acid sequence, the amino acid sequence preferably further comprises up to (but not more than) 1 to 30, more preferably 1 to 20, most preferably 1 to 15 amino acids; and/or
(d) a long peptide, i.e., a compound, construct, or polypeptide, the amino acid sequence of which comprises at least one antigen peptide as defined in items (a) to (c) above; and/or
(e) a tandem peptide, i.e., a compound, construct, or polypeptide comprising or consisting of at least two identical or different peptide sequences of at least one HLA-A antigen peptide and one HLA-A and/or HLA-B antigen peptide, preferably both as defined in items (a) to (c), in which the HLA antigen peptides are optionally linked to each other by suitable linkers (so-called oligopeptides); and/or

Very preferably, HLA-A antigen peptides are as defined in (b), (d) and (e), respectively. Also in the embodiment according to (e), the HLA-A antigen peptides are preferably defined as described under (a) or (d). In the case that the HLA-A antigen peptides as defined under (e) are linked to each other via a linker, suitable linkers are known to the skilled person from the prior art.

That one of the aforementioned amino acid sequences, in particular as defined in items (a) to (c), may further comprise up to (but not more than) 1 to 30, more preferably 1 to 20, most preferably 1 to 15 amino acids in addition to the HLA-A antigen peptide, has the advantage that the stability of the HLA-A antigen peptides is increased thereby. In particular, this increases the stability after administration to a subject, as they may be undergoing partial degradation from the C- and/or N-terminus after application.

In a particular embodiment of the present invention, one of the aforementioned amino acid sequences, in particular as defined in items (a) to (c), comprises of preferably up to (but not more than) 3 HLA-B antigen peptides, more preferably up to two HLA-B peptides, and further comprise up to (but not more than 1 to 20, more preferably 1 to 5 most preferably 1 to 10 amino acids. This has the particular advantage that more than one viral epitope can be targeted with a single amino acid sequence in accordance to the present invention, thus increasing a potential B/T cell response.

The use of compounds, constructs or polypeptides as defined under (e) according to the invention, which consist of at least two identical or different peptide sequences of HLA antigen peptides, has the further advantage that the longer amino acid sequences of the compounds, constructs, proteins or polypeptides result in a longer retention time in the tissue of the subject after application, whereby the compounds, constructs, proteins or polypeptides after application to the subject can, for example by endogenous enzymes into smaller fragments (pharmaceutically active form comprising at least 6 to 12 amino acids) which have a biologically desired function in the sense of the invention. That is, the individual fragments of the oligopeptide exhibit activity as HLA-A and/or HLA-B antigen peptides and thus contribute to the activation of T cells.

In a particular embodiment, any HLA-A peptide sequence may be a humanized and/or sequence optimized sequence as further described herein.

An “HLA-B antigen peptide corresponding to MHC class I complexes” is defined herein as an “HLA-B antigen peptide of the invention” (also called “HLA-B antigen peptide”) or “amino acid sequence of the invention” (as defined herein), which comprises the following:
(a) an amino acid sequence preferably consisting of 6 to 12 amino acids, more preferably consisting of 7 to 10 amino acids, and most preferably consisting of 8 or 9 amino acids corresponding to MHC class I complexes which is similar or more preferably identical to a protein encoding RNA and/or DNA sequence in the genome of a virus causing a viral infectious disease, preferably from the transcriptome encoding the proteins of the virus presented on the surface of the virus, more preferably being part of the viral epitope.
(b) preferably, the HLA-B antigen peptide is present in the pharmaceutical composition according to the invention at a concentration, as defined above for the HLA-A antigen peptide, of at least 100 μg, alternatively in absolute concentration of preferably at least 600 μg relative to the volume of the pharmaceutical composition to be applied.

Preferably, the pharmaceutical composition contains (per vial) 600 μg×12=7,200 μg of antigen peptides.

Preferably, the HLA-B antigen peptides are selected as defined above in point (b).

According to a particularly preferred embodiment, an “HLA-B antigen peptide”, preferably comprising the following scaffold sequence:
(a) an amino acid sequence comprising or consisting of an amino acid sequence selected from the group consisting of SEQ ID NOs: 2, 8, 10-15, 17, 20, 24-26, 28, 31-32, 35, 37, wherein when the amino acid sequence comprises an aforementioned amino acid sequence, the amino acid sequence preferably further comprises up to (but not more than) 1 to 30, more preferably 1 to 20, most preferably 1 to 15 amino acids; and/or
(b) an antigen peptide comprising or consisting of an amino acid sequence having less than 100% sequence identity or similarity to the native HLA-B antigen peptide, such as having at least 85%, more preferably at least 90%, sequence identity (as defined herein) to an amino acid sequence selected from the group consisting of SEQ ID NOs: 2, 8, 10-15, 17, 20, 24-26, 28, 31-32, 35, 37, wherein when the amino acid sequence comprises an aforementioned amino acid sequence, the amino acid sequence preferably further comprises up to (but not more than) 1 to 30, more preferably 1 to 20, most preferably 1 to 15 amino acids; and/or
(c) an antigen peptide having an amino acid sequence comprising or consisting essentially of only one amino acid substitution relative to the amino acid sequence(s) selected from the group consisting of SEQ ID NO: 2, 8, 10-15, 17, 20, 24-26, 28, 31-32, 35, 37, wherein when the amino acid sequence comprises an aforementioned amino acid sequence, the amino acid sequence preferably further comprises up to (but not more than) 1 to 30, more preferably 1 to 20, most preferably 1 to 15 amino acids; and/or
(d) a long peptide, i.e., a compound, construct, or polypeptide whose amino acid sequence comprises at least one antigen peptide as defined in items (a) through (c) above; and/or
(e) a tandem peptide, i.e. a compound, construct, or polypeptide comprising or consisting of at least two identical or different peptide sequences of at least one HLA-B antigen peptide and one HLA-A and/or HLA-B, preferably both as defined in items (a) to (c), in which the HLA antigen peptides are optionally connected to each other by suitable linkers (so-called oligopeptides).

Very preferably, HLA-B antigen peptides are as defined in (a), (b), (c) and (e), respectively. Also in the embodiment according to (e), the HLA-B antigen peptides are preferably defined as described in items (a) through (c). In the case that the HLA-B antigen peptides as defined under item (e) are linked to each other via a linker, suitable linkers are known to the person skilled in the art from the prior art.

That one of the aforementioned amino acid sequences, in particular as defined in items (a) to (e), may further comprise up to (but not more than) 1 to 30, more preferably 1 to 20, most preferably 1 to 15 amino acids in addition to the HLA-B antigen peptide, has the advantage that the stability of the HLA-B antigen peptides is increased thereby. In particular, this increases the stability after administration to a subject, as they may be undergoing partial degradation from the C- and/or N-terminus after application.

In a preferred embodiment of the current invention polypeptides with 1 to 20 amino acids in length are especially preferred as antigen polypeptides in the spirit of the current invention since they are more economic in the preparation and synthesis than longer peptides.

In an especially preferred embodiment of the current invention polypeptides with 9 to 15 amino acids in length are especially preferred as antigen polypeptides in the spirit of the current invention since they are more economic in the preparation and synthesis than longer peptides.

In a particular embodiment of the present invention, one of the aforementioned amino acid sequences, in particular as defined in items (a) to (c), comprises of preferably up to (but not more than) 3 HLA-B antigen peptides, more preferably up to two HLA-B peptides, and further comprise up to (but not more than 1 to 20, more preferably 1 to 15 most preferably 1 to 10 amino acids. This has the particular advantage that more than one viral epitope can be targeted with a single amino acid sequence in accordance with the present invention, thus increasing a potential B/T cell response. The use of compounds, constructs, proteins or polypeptides as defined under (e) according to the invention, which consist of at least two identical or different peptide sequences of HLA antigen peptides, has the further advantage that the longer amino acid sequences of the compounds, constructs, proteins or polypeptides result in a longer retention time in the tissue of the subject after application, whereby the compounds, constructs, proteins or polypeptides after application to the subject can, e.g. by endogenous enzymes into smaller fragments (pharmaceutically active form, comprising at least 6 to 17 amino acids), which have a biologically desired function in the sense of the invention, by macrophages and dendritic cells. That is, the individual fragments of the oligopeptide exhibit activity as HLA-A and/or HLA-B antigen peptides and thus contribute to the activation of T cells.

In a particular embodiment, any HLA-B peptide sequence may be a humanized and/or sequence optimized sequence as further described herein.

In a particularly preferred embodiment, the HLA-A and/or HLA-B antigen peptides of the present invention are selected in such a way, that they match or correspond to at least one viral infectious disease epitope, which are highly conserved across different virus variants, preferably in at least 2 different virus variants, more preferably in at least 4 different virus variants, most preferably in at least 6 different virus variants. This has the technical effect, that the combination preparation relating to the aforementioned antigen peptides is less susceptible against RNA and/or DNA mutations in new strains and variants of the viral infectious disease and thus allowing for a better (broadband) immunization. Correct sequence matching can be achieved by comparing the amino acid sequences of the different viral variants and identifying the regions, that are (i) identical to each other or (ii) having an amino acid sequence comprising or consisting essentially of only one amino acid substitution relative to each other or (iii) having at least 80%, 85%, 90%, 95%, 96%, 97%, 98%, or 99% identity to each other and screening these regions to identify appropriate HLA-A and/or HLA-B antigen sequences. It has been found to be particularly advantageous to compare not only different variants of a subspecies of the viral infectious diseases among themselves (e.g., different mutants the virus) but rather different variants within the family, and/or at least within the genus, but at least different mutants within a subgenus. This allows the HLA-A and/or HLA-B antigen peptides and/or antigen polypeptides according to the invention to be used not only for the treatment of a specific virus types, but moreover for the use in the broad-spectrum immunization, preferably for use in the therapeutic or prophylactic treatment of a family-spanning, preferably at least subfamily-spanning, particularly preferably genus-spanning, especially subgenus-spanning viral infectious disease.

In an aspect of the current invention which discloses a particularly preferred embodiment, the HLA-A and/or HLA-B antigen peptides of the present invention are selected in such a way, that they match or correspond to coronavirus, preferably betacoronaviruses, more preferably sarbecovirus, most preferably SARS-COV-2 epitope regions, preferably spike S1 domain, S2 domain, nucleocapsid protein, envelope protein and/or ORF1ab polyprotein, which are highly conserved across different virus variants, preferably in at least 2 different virus variants, more preferably in at least 4 different virus variants, most preferably in at least 6 different virus variants. This has the technical effect, that the combination preparation relating to the aforementioned antigen peptides is less susceptible against RNA mutations in new strains and variants of the SARS-COV-2 epitope, such as the Alpha (B.1.1.7), or Beta (B.1.351), or Gamma (P.1), or Delta (B.1.617.2) and/or Omicron variant (B.1.1.529) and thus allowing for a better (broadband) immunization. Correct sequence matching can be achieved by comparing the amino acid sequences of the different coronavirus variants and identifying the regions, preferably with respect to the spike S1 domain, S2 domain, nucleocapsid protein, envelope protein and/or ORF1ab polyprotein, that are (i) identical to each other or (ii) having an amino acid sequence comprising or consisting essentially of only one amino acid substitution relative to each other or (iii) having at least 80%, 85%, 90%, 95%, 96%, 97%, 98%, or 99% identity to each other and screening these regions to identify appropriate HLA-A and/or HLA-B antigen sequences. It has been found to be particularly advantageous to compare not only different variants of a subspecies of coronavirus among themselves (e.g., different mutants of SARS-CoV 2) but rather different variants within the family (coronavirus), at least within the genus (betacoronaviruses), but at least different mutants within a subgenus (sarbecovirus). This allows the HLA-A and/or HLA-B antigen peptides and/or antigen polypeptides according to the invention to be used not only for the treatment of a specific virus types (such as SARS-COV 1, SARS-COV 2, MERS-COV), but moreover for the use in the broad-spectrum immunization, preferably for use in the therapeutic or prophylactic treatment of a family-spanning, preferably at least subfamily-spanning, particularly preferably genus-spanning, especially subgenus-spanning coronavirus infectious disease.

In another aspect of the current invention which discloses a particularly preferred embodiment, the nucleocapsid protein, for example of the SARS-COV 2 was aligned against 5 different coronaviruses within the coronavirus family, preferably with a (multiple) sequence alignment tool, such as COBALT tool (Human coronavirus 229E, UNIPROT reference: P15130; Human coronavirus OC43, UNIPROT reference: P33469, Human coronavirus HKU1 (isolate N5), UNIPROT reference: QOZME3; Human coronavirus HKU1 (isolate N1), UNIPROT reference: Q5MQC6; and severe acute respiratory syndrome coronavirus 2, UNIPROT reference: Q6S8E1) according to a conservation algorithm based on the relative entropy threshold of the residue. SARS-COV-2 nucleocapsid protein positions 59-84, 86-96, 99-123, 129-142, 155-170, 284-320, 321-336, 342-360 are highly conserved. These regions are highly preferable as target for HLA-A and/or HLA-B antigen peptide, preferably as described herein, corresponding to MHC class I complexes in accordance with the present invention.

In an alternative embodiment of the invention, the SARS-COV-2 spike protein was aligned against 8 different coronaviruses within the coronavirus family (Human coronavirus 229E, UNIPROT reference: P15423; Human coronavirus OC43, UNIPROT reference: P36334, Human coronavirus HKU1 (isolate N5), UNIPROT reference: QOZME7; Human coronavirus HKU1 (isolate N1), UNIPROT reference: Q5MQD0; Severe acute respiratory syndrome coronavirus, UNIPROT reference: P59594; Human Coronavirus NL63, UNIPROT reference: Q6Q1S2; MERS-COV, UNIPROT reference: K9N5Q8; and Human coronavirus HKU1 (isolate N2), UNIPROT reference: Q14EB0). SARS-COV-2 spike protein positions 152-211, 470-503, 534-570, 769-847, 939-1063 and 1176-1273 are highly conserved. These regions are highly preferable as target for HLA-A and/or HLA-B antigen peptide, preferably as described herein, corresponding to MHC class I complexes in accordance with the present invention.

Preferably, a monovalent amino acid sequence (or a polypeptide comprising only an amino acid sequence of the invention) used in the invention binds to a T cell receptor of endogenous T cells with a 3-fold increased specific affinity compared to the corresponding wild-type HLA peptide sequence. It should be noted that “can specifically bind to” and “specifically binds to” are used synonymously herein and refer to the ability to bind specifically to the corresponding specified entity.

It is possible to combine amino acid sequences belonging to different classes used in the invention into a single polypeptide of the invention. In particular, it has been demonstrated that the combination of HLA-A and/or HLA-B antigen peptides in a single polypeptide of the invention have unique binding properties (cf. FIG. 3).

By one skilled in the art, the specific activity (KD) of the polypeptides of the invention comprising more than one component of the amino acid sequence of the HLA-A and/or HLA-B antigen peptides corresponding to the class I MHC complexes can be determined according to one of the detection methods described above/below, wherein the compounds, constructs, proteins or polypeptides of the invention preferably have a specific activity similar to the specific activity of each of their components; i.e., a specific activity similar to the specific activity of each of the (individual components of the) amino acid sequences of MHC class I contained in the compounds, constructs, or polypeptides of the invention. Some specific, but not limiting, examples of the above preferred compounds, constructs, or polypeptides are compounds, constructs, or polypeptides that either.
i) a wild-type HLA antigen peptide corresponding to class I MHC complexes (HLA-A and/or HLA-B or HLA-C peptide, respectively); or
ii) an antigen corresponding to class I MHC complexes (HLA-A and/or HLA-B antigen) and having an amino acid sequence selected from the group consisting of SEQ ID NOs: 1-37; 59-79 or
iii) an antigen corresponding to class I MHC complexes (HLA-A and/or HLA-B antigen) and/or class II MHC complexes having an amino acid sequence having at least 80% sequence identity (as defined herein) with an amino acid sequence selected from the group consisting of SEQ ID NOs: 1-37, 59-79, more preferably 2-37, 59-79; or
iv) an amino acid sequence comprising or consisting essentially of only one amino acid substitution relative to the amino acid sequence selected from the group of SEQ ID NOs: 1-37, 59-79, more preferably 2-37, 59-79; or
(v) compounds, constructs, proteins or polypeptides consisting of at least two identical or different HLA antigen peptide sequences in which the HLA peptides are optionally linked together by suitable linkers;
   or a suitable combination thereof.

It should be noted that the last preceding paragraphs also generally apply to all amino acid sequences of the invention comprising one or more amino acid sequences for the HLA antigen peptides defined above.

In one embodiment of the current invention, all the above HLA antigen peptides corresponding to Class I and Class II MHC complexes can be used and are effective for treating a coronavirus infectious disease as disclosed herein, particularly for treating SARS-COV-2.

Some specific, but not limiting, examples of such compounds, constructs, proteins or polypeptides of the invention are set forth, for example, in Tables 1-4 or are apparent to those skilled in the art based on the present disclosure.

According to another preferred embodiment of the present invention, the HLA antigen peptide is present (in each case) as a single-membered amino acid sequence, i.e., not as an element of compounds, constructs, or polypeptides consisting of at least two identical or different amino acid sequences for HLA antigen peptides, in which the HLA antigen peptides are connected to each other by suitable linkers.

It is also part of the present invention that an amino acid sequence of the invention can bind to two or more class I MHC complexes, epitopes, components, domains or subunits of a class I MHC complex. In such a case, the MHC complexes, epitopes, components, domains or subunits of an MHC complex to which the amino acid sequences and/or polypeptides of the invention bind may be substantially the same or different (and in the latter case, the amino acid sequences and polypeptides of the invention may bind to such different complexes, epitopes, components, domains or subunits of a class I MHC complex, including combinations thereof, with an affinity and/or specificity that may be the same or different).

It is also expected that the polypeptides of the invention will generally bind to all naturally occurring or synthetic analogs, variants, mutants, components and fragments of a class I MHC complex. Also in such a case, the amino acid sequences and polypeptides of the invention may bind to such analogs, variants, mutants, components and fragments with an affinity and/or specificity equal to or different from the affinity and specificity with which the amino acid sequences of the invention bind to the wild-type of the class I MHC complex.

It is also within the scope of the present invention that the amino acid sequences and polypeptides of the invention bind to some analogs, variants, or mutants of a class I MHC complex, but not to other.

### Formulation of the Pharmaceutical Combination

According to a particularly preferred embodiment of the present invention, the pharmaceutical composition consists exclusively of a carrier liquid (preferably water, a pharmaceutically acceptable saline solution and/or pharmaceutical DMSO. Such compositions of carrier fluids are known to those skilled in the art and are, for example, in the range of preferably 5-30% DMSO and 70-95% water. Particularly preferably, the percentage of DMSO is 25% and that of water 75%. A pharmacologically effective amount of preferably at least one and particularly up to 12 peptide complexes is dissolved or suspended in the carrier liquid and mixed with an adjuvant selected from the following 5 adjuvants. An adjuvant preferably comprises CpG (oligonucleotide of cytidine and guanosine), c-di-AMP (bis-(3′,5′)-cyclic-adenosine), alum (usually a mixture of aluminum sulfate, potassium or sodium hydroxide, and phosphate), Pam3Cys-GDPKHPKSF N-palmitoyl-S-[2,3-bis(palmitoyloxy)-(2R)-propyl]-(R)-cysteinyl-GDPKHPKSF), and poly-IC. Other suitable adjuvants and compositions of carrier fluids are known to the skilled person from the prior art.

The pharmaceutical composition and suitable dosage forms for application of the pharmaceutically active antigen polypeptides and/or HLA antigen peptides are prepared according to standard procedures known in the prior art and are readily applicable to any new or improved process for their preparation.

Another object of the present invention is the (medicament) formulation in their dosage form containing the combination of active ingredients according to the invention and optionally further active ingredients and/or excipients.

Preferred drug formulations are transdermal therapeutic systems, solutions, injections, emulsions, suspensions, easily reconstitutable dry preparations, powders, or sprays. Particularly preferred drug formulations are injections or solutions.

Alternatively, the drug formulation is present in a suitable application device, preferably as a lyophilizate in a syringe, which allows in situ reconstitution with a pharmaceutically acceptable solution (e.g., saline).

In one embodiment, the antigen polypeptide corresponding to at least one HLA antigen peptide corresponding to MHC class I complexes or the amino acids is formulated or is to be formulated as a liquid, a solid, or a combination thereof.

Preferably, the (medicament) pharmaceutical composition/formulation according to the invention is suitable for intravenous, intramuscular, subcutaneous, intrathecal, epidural, transdermal or nasal administration, with (medicament) formulations for subcutaneous or intravenous administration being particularly preferred. In a particularly preferred embodiment of the invention, the pharmaceutical composition is applicated subcutaneous. This has the technical advantage of reduced antigen peptide degradation and a depot forming effect and thus, the antigen polypeptide is slowly delivered to the subject.

In one embodiment, the HLA antigen peptide corresponding to MHC class I complexes and/or the antigen polypeptides are formulated or are to be formulated for injection.

In one embodiment, the HLA antigen peptide corresponding to MHC class I complexes and/or the antigen polypeptides are formulated or are to be formulated for intramuscular administration.

In one embodiment, the pharmaceutical composition/formulation is a medical composition. In one embodiment, the pharmaceutical composition/formulation is a vaccine.

In one embodiment, the HLA antigen peptide and/or the antigen polypeptide is formulated or is to be formulated as particles.

In one preferred embodiment, each single HLA antigen peptide described herein, preferably each antigen polypeptide, if it is part of the pharmaceutical composition, for the therapeutic or prophylactic treatment of a viral infectious disease, especially for the prophylactic treatment, may be administered at an absolute concentration per dose of the pharmaceutical composition (i.e., administration dose) in a range from 10 to 1,000 μg, preferably from 100 to 700 μg, preferably from 300 to 600 μg, such as up to 150 μg, up to 200 μg, up to 250 μg, up to 350 μg, up to 400 μg, up to 450 μg, up to 500 μg or up to 550 μg may be administered. In one embodiment, the invention relates to a single dose administration. In one embodiment, the invention relates to the administration of a priming dose followed by one or more booster doses. The booster dose or the first booster dose may be administered 7 to 28 days or 14 to 24 days following administration of the priming dose.

In one preferred embodiment, each single HLA antigen peptide described herein, preferably each antigen peptide, if it is part of the pharmaceutical composition, for the therapeutic or prophylactic treatment of a viral infectious disease, especially for the therapeutic treatment, may be administered at an absolute concentration per dose of the pharmaceutical composition (i.e., administration dose) of at least 100 μg and particularly preferably at least 500 μg, alternatively preferably at an absolute concentration of at least 600 μg, i.e., containing at least in the range of 700 to 1,200 μg, preferably from 800 to 1,200 μg, is particularly preferred, as this greatly intensifies the therapeutic effect. This is particularly advantageous if the immune system of the subject to be treated is already weakened by other pre-disease, e.g., cardiovascular disease, diabetes, chronic cardiopulmonary disease, hypertension, chronic renal disease, chronic pulmonary disease, AIDS/HIV, any malignancy, lymphoma or metastatic solid tumor and possibly associated pretreatment thereof, or if the subject already suffers from a viral infection. Irrespective of the above, an increased absolute concentration per HLA antigen peptide is also preferred, as this substantially minimizes the influence of degradation of the HLA antigen peptides (e.g., by ligases) after their administration to the subject.

In a preferred embodiment, each single HLA antigen peptide described herein, preferably each antigen peptide containing amino acid sequences corresponding to at least two viral infectious diseases, if it is part of the pharmaceutical composition, for the therapeutic or prophylactic treatment of a viral infectious disease, especially for the therapeutic treatment, may be administered at an absolute concentration per dose of the pharmaceutical composition (i.e., administration dose) of at least 100 μg and particularly preferably at least 500 μg, alternatively preferably at an absolute concentration of at least 600 μg, i.e., containing at least in the range of 700 to 1,200 μg, preferably from 800 to 1,200 μg, is particularly preferred, as this greatly intensifies the therapeutic effect and allows for simultaneous immunization against at least two different viral infectious diseases. In a particularly preferred embodiment, at least two different HLA antigen peptides matching different viral epitopes (preferably from different viral infectious diseases) as described herein, especially preferably antigen polypeptides are present in the pharmaceutical composition allowing for immunization against at least two different viral infectious diseases.

In one embodiment, administration of the pharmaceutical composition as described herein, more preferably administration of the pharmaceutical composition with an administration dose as described herein, most preferably after single dose administration, particularly after second dose administration, most particularly after third dose administration of the pharmaceutical composition with an administration dose as described herein results in an IFNγ activity in the blood serum of the subject in a range of at least 50 to 500, more preferably a range of at least 100 to 400, most preferably a range of 100 to 250, determined via IFNγ ELISpot analysis as described herein.

In one embodiment, administration of the pharmaceutical composition as described herein, more preferably administration of the pharmaceutical composition with an administration dose as described herein, most preferably after single dose administration, particularly after second dose administration, most particularly after third dose administration of the pharmaceutical composition with an administration dose as described herein results in an IFNγ cell count per 100.000 cells in the blood serum of the subject in a range of at least 25 to 800 cells, more preferably a range of at least 50 to 600 cells, most preferably a range of 50 to 500 cells, determined via IFNγ ELISpot analysis as described herein.

In one embodiment, the pharmaceutical composition/formulation is a kit. Thus, in one embodiment, the antigen peptide corresponding to MHC class I complexes or the antigen polypeptide and optionally the carrier liquid, a pharmaceutically acceptable adjuvant, carrier, diluent and/or excipient are in separate vials.

Methods known in the prior art for the preparation of pharmaceutical compositions or dosage forms are found in, for example, “Remington's Pharmaceutical Sciences”. Pharmaceutical compositions for parenteral administration may contain, for example, excipients, sterile water, or saline, polyalkylene glycols, such as polyethylene glycols, oils of plant, animal, or microbial origin, or hydrogenated naphthalenes. Biocompatible, biodegradable lactide polymer, lactide/glycolide copolymers, or polyoxyethylene-polyoxypropylene copolymers can be used to control the release of the compounds. Other potentially useful parenteral delivery systems for therapeutic anti-prion compounds, for example, include ethylene vinyl acetate copolymer particles, osmotic pumps, implantable infusion systems, and liposomes.

In other embodiments, the present invention also relates to a pharmaceutical composition, a kit (or parts thereof), a method for determining/identifying a pharmacologically active HLA antigen peptide corresponding to class I MHC complexes and/or antigen peptide, a method for preparing a formulation according to the invention, and the use of a formulation according to the invention for the preparation of a pharmaceutical composition for the treatment and prevention of a viral infectious disease.

Pharmaceutical compositions are prepared by dissolving or suspending the determined HLA antigen peptides and/or antigen polypeptides in a carrier fluid (i.e., a pharmacologically acceptable vehicle), optionally with the addition of other excipients such as wetting agents, dyes, permeation enhancers, resorption enhancers, preservatives, antioxidants, light stabilizers.

The carrier liquid is preferably selected from the group consisting of sodium chloride injection solution, Ringer's injection solution, isotonic dextrose, sterile water, dextrose solution, lactated Ringer's injection solution, distilled water or mixtures thereof, for local injection.

It is particularly advantageous for good solubility of the determined HLA antigen peptides if their amino acid sequence has the lowest possible number of hydrophobic amino acids.

Preferably, dimethyl sulfoxide (DMSO), Diethylene glycol diethyl ether, ethanol, phosphatidylcholines, propylene glycol dipelargonates (DPPG), or glycolyzed ethoxylated glycerides are suitable permeation promoters.

According to a preferred embodiment of the present invention, the pharmaceutical composition comprises water, a pharmaceutically acceptable saline solution, and/or DMSO. For example, pharmaceutical compositions comprising a mixture of, for example, 25% DMSO and 75% water are suitable for application.

### Process for the Determination of Pharmaceutically Active Peptides

Bioinformatic processes can be used to recognize and determine regions in epitopes which correspond to HLA antigen peptides for use in a pharmaceutical composition in accordance with the current invention. Thus, T and B cells identify these regions, and it is it is also an outstanding achievement of the inventors to have found a generalized way to determine such regions for the use in a pharmaceutical composition for use in a vaccine for viral infectious diseases.

In an alternative embodiment of the current invention a method which uses bioinformatic models to determine these aforementioned regions can be used to find parts in a virus epitope, preferably a coronavirus, more preferably a betacoronaviruses, especially preferably a sarbecovirus, most preferably SARS-COV-2 epitope regions.

Also encompassed by the present invention is a method for determining pharmaceutically active HLA antigen peptides and/or antigen polypeptides for use in the treatment or prophylaxis of at least one viral infectious disease or in a pharmaceutical composition according to the invention, said method comprising the following steps:

(a) Bioinformatic expert system characterization and prediction of immunogenic, ADE-avoiding and B/T cell response balancing viral epitope peptides. Bioinformatic analyses of mechanistic ADE hypotheses are used to clarify open questions about molecular mechanisms of ADE. Bioinformatic 3D modeling and binding prediction can test the effect of different antibodies on the conformational change of the of the viral epitopes and select peptides of the viral infectious disease that are less likely to trigger ADE. Artificial intelligence is used for information processing, and the mathematical modeling and biotechnological advancements based on it are used for experimental analysis on the causality of a pharmaceutical composition of the vaccine. This allows existing knowledge, including that within the existing viral infectious diseases epitope collections, to be extracted from the extensive literature and tested for gaps through modeling: Knowledge gaps can be narrowed by simulation-based hypothesis testing to the point where experimental testing of the remaining variants becomes feasible.

ADE and B/T cell response balancing viral epitope peptides was made using a variety of in silico methods, 3D modeling, and binding prediction test the effect of different antibodies on the conformational change of the viral epitopes and select peptides of the viral infectious disease that are less likely to trigger ADE. Artificial intelligence such as the neural network NNAlign used in the publicly available NetMHC-4.0 server is used for information processing, and the mathematical modeling and biotechnological advancements based on it are used for experimental analysis on the causality of a pharmaceutical composition of the vaccine. From the dataset of the viral infectious virus genome (in an aspect of the current invention, a virus epitope related to SARS-COV-2, especially Wuhan HU-1, Wuhan HU-1, nucleoprotein, UNIPROT Reference ID sequence: PODTC9, and spike protein, UNIPROT Reference ID sequence: PODTC2) nonameric variants involving amino acid exchange were determined with respect to 12 of the highest affinities (specific activity toward T cell receptor [nM]) based on the patient's alleles. Also, polymers of 17 amino acids (oligopeptides) were determined under the affinity criterion for this purpose. This selection criterion is used to algorithmically determine the probability of presenting the respective antigen peptide corresponding to the MHC complexes in vivo on the corresponding MHC complexes (a 15 second requirement for a possible cellular immune response).

b) Mathematical model for causality analysis of the relationship between vaccine assembly and ADE.

The likelihood of certain peptides being presented as epitopes on MHC-I and eliciting a T cell response can be predicted by various algorithms, each of which has low to moderate specificity. Similarly, sequence-based predictions of 3D protein structures with a few angstroms of resolution are possible if the respective sequence forms stable secondary structures and has at least sufficient homology to sequences with known structure. Bioinformatic predictability combines the evaluation of individual algorithms with the optimization of the parameters of the algorithms as well as the combination of algorithms. In addition, artificial intelligence optimizes the identification of significant predictive information and association.

(c) An AI and machine learning-based system for recognizing information links in scientific texts, integrating tables and figures during evaluation, and identifying relevant answers to specific questions.

Natural language processing (NLP) such as Natural Language Toolkit (NLTK) is used to extract further information to reinforce the predictions from Steps (a) and (b). The combination of artificial intelligence and machine learning methods, for example “Natural Language Processing” (NLP), structure recognition, deep learning etc., is used to learn patterns that allow information linkage to be identified or that allow tables and figures to be included in the evaluation. Thus, relevant information on molecular and cellular interaction networks between viral infectious diseases and the host can be extracted from the total amount of scientific literature and related to ADE.

Mathematical modeling approaches are nowadays successfully used to study cellular processes such as signal transduction, gene regulation and metabolism. Information on ADE collected using artificial intelligence is translated into a mathematical model that maps the molecular mechanisms.

(d) Biotechnology platforms for high-resolution, conformation-specific viral antibody characterization, cellular immune response characterization, and vaccine design optimization.

In the development of peptide immunizations, both B-cell activating and different T-cell activating peptides can be heuristically designed. Subsequently, screening with high-resolution analysis of antibodies induced by the vaccines is required to determine both neutralization capacity and epitope specificity.

In an alternative embodiment of the present invention related to coronaviruses, the amino acid sequences of the HLA antigen peptides of the SARS-COV-2 corresponding to the MHC complexes (nonamers) with the highest allelic affinities (specific activity towards T cell receptor [nM]) were first selected from the dataset of the SARS-COV-2 virus proteins (nucleoprotein, UNIPROT Reference ID sequence: PODTC9, and spike protein, UNIPROT Reference ID sequence: PODTC2) based on the amino acid sequences of the respective proteins. The highest conservated regions in relation with other human coronaviruses and non-variant mutations were selected from proteins with a combination of different bioinformatic methods to further analyze. To this scope, the Nucleocapsid protein was aligned against 5 coronaviruses with COBALT tool (Human coronavirus 229E, UNIPROT reference: P15130; Human coronavirus OC43, UNIPROT reference: P33469, Human coronavirus HKU1 (isolate N5), UNIPROT reference: QOZME3; Human coronavirus HKU1 (isolate N1), UNIPROT reference: Q5MQC6; and severe acute respiratory syndrome coronavirus 2, UNIPROT reference: Q6S8E1) according to a conservation algorithm based on the relative entropy threshold of the residue. SARS-COV-2 nucleocapsid protein positions 59-84, 86-96, 99-123, 129-142, 155-170, 284-320, 321-336, 342-360 are highly conserved. These regions are used for further binding affinity prediction. SARS-COV-2 spike protein was aligned against 8 coronaviruses (Human coronavirus 229E, UNIPROT reference: P15423; Human coronavirus OC43, UNIPROT reference: P36334, Human coronavirus HKU1 (isolate N5), UNIPROT reference: QOZME7; Human coronavirus HKU1 (isolate N1), UNIPROT reference: Q5MQD0; Severe acute respiratory syndrome coronavirus, UNIPROT reference: P59594; Human Coronavirus NL63, UNIPROT reference: Q6Q1S2; MERS-COV, UNIPROT reference: K9N5Q8; and Human coronavirus HKU1 (isolate N2), UNIPROT reference: Q14EB0). SARS-CoV-2 spike protein positions 152-211, 470-503, 534-570, 769-847, 939-1063 and 1176-1273 are highly conserved. then, the selection criterion is used to algorithmically determine the probability with which the respective HLA antigen peptide is presented in vivo on the corresponding MHC complexes of the SARS-COV-2 virus (a first prerequisite for a possible T cell immune response). This has the advantage that regions with the highest conservated character have a lesser probability of mutations across new coronavirus, such as SARS-COV-2 virus, and thus allowing for an immunization against new variants.

According to a preferred embodiment of the present invention, a method for determining pharmaceutically active HLA antigenic peptides and/or antigenic polypeptides for use in the treatment or prophylaxis of a viral infectious disease, or in a pharmaceutical composition according to the invention is used, said method comprising the following steps (a)-(e):
(a) determining the amino acid sequence(s) from at least one viral genome, preferably for parts of the proteome expressed on the viral surface proteins, more preferably being a part of the viral epitope(s),
(b) comparing the amino acid sequence(s), preferably those determined according to (a), with those of at least one other related virus (preferably of the same family, more preferably of the same subfamily, especially preferably from the same genus, most preferably from the same species) and determining the conserved regions in the viral genomes,
(c) determining amino acid sequence(s) corresponding to MHC class I and/or class II complexes and/or matching to B-cells, preferably belonging to conserved viral epitope region(s), more preferably those determined according to (b),
(d) developing synthetic antigen polypeptide(s) containing the amino acid sequences, preferably selected according to (c) and determining the physiological and physicochemical properties,
(e) creating a composition of at least one, more specific at least two, more preferably at least three, most preferably up to twelve antigen polypeptide(s), preferably according to (d), while considering HLA allele distribution of the worldwide population.

According to one especially preferred embodiment of the present invention, a method for determining pharmaceutically active HLA antigenic peptides and/or antigenic polypeptides for use in the treatment or prophylaxis of preferably at least two, more preferably at least three, especially preferably at least four, most preferably up to five different viral infectious disease(s), or in a pharmaceutical composition according to the invention is used, said method comprising the following steps (a)-(e):
(a) determining the amino acid sequence(s) from preferably at least two, more preferably at least three, especially preferably at least four, most preferably up to five different viral infectious disease(s) genome, preferably for parts of the proteome expressed on the viral surface proteins, more preferably being a part of the viral epitope(s),
(b) comparing the amino acid sequence(s), preferably those determined according to (a), with those of at least one other related virus per selected genome of (a) (preferably of the same family, more preferably of the same subfamily, especially preferably from the same genus, most preferably from the same species) and determining the conserved regions in the viral genomes,
(c) determining amino acid sequence(s) corresponding to MHC class I and/or class II complexes and/or matching to B-cells, preferably belonging to conserved viral epitope region(s), more preferably those determined according to (b),
(d) developing synthetic antigen polypeptide(s) containing the amino acid sequences, preferably selected according to (c) and determining the physiological and physicochemical properties,
(e) creating a composition of at least one, more specific at least two, more preferably at least three, most preferably up to twelve antigen polypeptide(s), preferably according to (d), while considering HLA allele distribution of the worldwide population.

In preferred embodiment of the current invention, application of the above method has the technical advantage to find combination preparations containing antigen polypeptides allowing for an immunization against multiple viral infectious diseases while also being more resistant against viral mutations due to the determination of the conserved regions.

According to an alternative embodiment of the present invention related to a coronavirus, a method for determining pharmaceutically active HLA antigenic peptides and/or antigenic polypeptides for use in the treatment or prophylaxis of a viral infectious disease, such as a coronavirus, preferably a betacoronavirus, especially preferably a sarbecovirus, most preferably the SARS-COV-2 virus, or in a pharmaceutical composition according to the invention is used, said method comprising the following steps (a)-(e):
(a) determining the amino acid sequence from at least one viral genome sequencing proteins containing viral epitopes presented on the viral surface, preferably from the SARS-COV-2 wild type, more preferably of a SARS-COV-2 mutant with risk of circumventing existing immunization efforts, for example from prior infections or immunization against a other strain of the virus,
(b) comparing the amino acid sequence(s) of (a) with those of at least one other related virus and determining the conserved regions in the viral genomes, more preferably with at least three, most preferably with at least five related viral epitopes, where related viruses are for example a wild type, a mutant, a variant, or another genetically related virus,
(c) determining amino acid sequence(s) corresponding to MHC class I and/or class II complexes and/or matching to B-cells, preferably belonging to a conserved viral epitope region(s) according to (b),
(d) developing synthetic antigen polypeptides containing the amino acid sequences from (c) and determining the physiological and physicochemical properties,
(e) creating a composition of at least one, more specific at least two, more preferably at least three, most preferably up to twelve artificial antigen polypeptides, preferably according to (d), while considering HLA allele distribution of the worldwide population.

a) In one embodiment of the current invention, the transcriptome of the viral sample is used to determine any DNA sequences transcribed into mRNA sequences and to quantify the mRNA sequences. Viral epitopes are identified.

Particularly preferably, determination of any viral protein which is part of the viral epitope is performed by ultra-high performance liquid chromatography 5 (UHPCL) in combination with ESI mass spectrometry (MS) and compared with libraries of known epitopes, for example the Immune Epitope Database (IEDB). Preferably, the generation of the transcriptome is performed by RT-PCR, followed by 10 DNA microarray or DNA sequencing.

b) In a preferred embodiment of the present invention, the conservative regions in the viral epitope are first determined by using a sequence comparison tool, for example MUSCLE (Multiple Sequence Comparison by Log-Expectation) or for example COBALT, or another suitable algorithm known to the skilled person. It is aligned according to a conservation algorithm based on the relative entropy threshold of the residue. The regions will be distinguished into highly conserved and less highly conserved based on the number of exchanges and substitutions in the amino acid sequence. In a preferred embodiment of the present invention, it is advantageous to use only highly conserved regions to make the immune response less susceptible to future mutations. In an alternative embodiment of the present invention, portions of less conserved regions are included to allow for longer amino acid sequences that can be matched to HLA antigen peptides and B cells, thereby allowing for broader HLA coverage and a stronger B cell and T cell immune response.

In an alternative embodiment, the portion of the target viral epitope, for example a viral nucleocapsid protein and/or a S1 and/or S2 spike protein, preferably corresponding to a viral infectious disease, such as a coronavirus, more preferably a betacoronavirus, most preferably a sarbecovirus, especially preferably the SARS-COV-2 virus, is compared with at least one other viral epitope of a related viral infectious disease, such as a wild type, a mutant, a variant or another genetically related virus, preferably at least two, especially preferably at least four, most preferably at least six, alternatively at least nine different viral epitopes corresponding to mutants or variants, as previously described. The conserved regions found in this step can be used in B-cell and T-cell binding prediction, which may lead to more robust and mutation-resistant immunization of the combination preparation.

In a preferred embodiment of the invention related to coronaviruses, the amino acid sequence of the nucleocapsid protein (in FASTA Format) (ID: PODTC9) of Severe Acute Respiratory Syndrome Coronavirus 2 (SARS-COV-2, 2019-nCOV; amino acids 1-419) were preferentially compared with up to 5 other coronavirus strains, particularly preferentially comparing a selection of the following variants: Human Coronavirus OC43 (ID: P33469), Human SARS Coronavirus (SARS-COV) (ID: P59594), Human Coronavirus 229E (HCoV-229E) (ID: P15130), human coronavirus HKU1 (HCoV-HKU1, isolate N5) (ID: QOZME3), human coronavirus HKU1 (HCoV-HKU1, isolate N1) (ID: Q5MQC6), and severe acute respiratory syndrome coronavirus (ID: Q6S8E1).

In an especially preferred embodiment of the invention, it was found by the inventors that the conserved regions of the epitope are particularly well suited for the development of a therapeutic/prophylactic preparation because mutations of the virus are not as frequent and are therefore less susceptible to overcoming immunization.

In one embodiment of the current invention, particularly preferred regions of amino acid sequence found in the SARS-COV-2 nucleocapsid protein by application of this method are the amino acids at positions 152-211, 470-503, 534-542 (570), 769-847, 939-1063, and 1176-1273.

In a preferred embodiment of the invention, the amino acid sequence of the spike S protein (ID: PODTC2) of the Severe acute respiratory syndrome coronavirus 2 (SARS-COV-2, 2019-nCOV; amino acids 1-1723) where compared with up to 8 other coronavirus strains, especially preferably: Human coronavirus NL63 (HCoV-OC43) (ID: Q6Q1S2), human coronavirus OC43 (ID: P33469), human SARS coronavirus (SARS-COV) (ID: P59594), human coronavirus 229E (HCoV-229E) (ID: P15423), middle east respiratory syndrome-related coronavirus (ID: K9N5Q8), human coronavirus HKU1 (HCoV-HKU1, isolate N5) (ID: Q0ZME3), human coronavirus HKU1 (HCoV-HKU1, isolate N1) (ID: Q5MQC6), human coronavirus HKU1 (HCoV-HKU1, isolate N2) (ID: Q14EB0) and severe acute respiratory syndrome coronavirus (ID: Q6S8E1).

In an especially preferred embodiment of the current invention it was found by the inventors that the conserved regions of the nucleocapsid are especially suitable for the development of a treatment/prophylactic preparation because mutations of the virus are not as frequent and therefore less susceptible for overcoming an immunization. Particularly preferred regions, found by application of this method in the SARS-COV-2 nucleocapsid protein, are the amino acids at the positions 152-211, 470-503, 534-542 (570), 769-847, 939-1063 and 1176-1273.

In an especially preferred embodiment of the invention, at least two amino acid sequences that match HLA antigen peptides are selected to match conserved regions in the viral epitope that encode different proteins. In a particularly preferred embodiment, at least two of these amino acid sequences are combined in an antigen polypeptide such that the sequences contained encode different proteins in the virus.

In a preferred embodiment of the present invention, novel viral variants, such as a mutant SARS-COV-2 viral variant, for example the alpha (B.1.1.7) and/or beta (B.1.351) and/or gamma (P. 1) and/or delta (B.1.617.2) and/or omicron (B.1.1.529) variants can be analyzed. In this regard, the conserved regions used in the determination of antigen polypeptides matching HLA antigen peptides according to the present invention can be compared to predict whether mutations alter the amino acid sequence in the conserved regions. Thus, potential resistance to immunization can be predicted and evaluated in silico.

c) According to one embodiment of the current invention, B-cell and T-cell epitope are aligned to match the conserved regions of the viral epitope, especially preferably according to b). The conserved regions as identified, for example with means described in b), can be analyzed to predict a strong B cell response. A sequence from the viral epitope can elicit a B-cell response if it binds to an immunoglobulin or antibody that elicits a B-cell response. The prediction is linear because the binding is based on the sequence of the amino acid sequence. The sequences of the conserved viral epitope can be aligned using a suitable tool or algorithm. Any means known to the skilled person can be used, preferably the use of the online servers BepiPred (or BCell IEDB), SVMtrip, ABCPred and BCPreds. In a preferred embodiment, linear prediction is performed using at least two, preferably three, and most preferably four of the above programs for B-cell epitope prediction. This provides the best results, as binding affinities may vary between the different methods. In a preferred embodiment of the present invention, the threshold for a B-cell epitope matching candidate is above 0.5 (percent value of accuracy of the ML model in matching a peptide to the epitope). In an even more preferred embodiment, a portion of the conserved region with a score of 0.8 and higher is considered a B-cell epitope very good match.

In a particularly preferred embodiment of the present invention, different amino acid sequence matches with a score of at least 0.5 from linear affinity matching are combined to form an antigen polypeptide suitable for matching multiple B cell epitopes. This has the advantage of allowing sequences to match multiple B-cell epitopes simultaneously, resulting in a more sophisticated and robust immunogenic response. A longer combination peptide is also less affected by point mutations in the amino acid sequence. The prediction methods do not provide the same binding predictions.

In a especially preferred embodiment of the current invention, when a combination of the above tools is used in a combination polypeptide, a better in vivo docking response can be achieved by targeting more than one potential B-cell binding site. Therefore, first every predicted peptide with a score higher than 0.5 (0.5 means specificity and sensitivity are higher than 0.5) is preselected. In a preferred embodiment of the current invention, these sequences are further refined, if they have been found equal or partially, preferably with more than 70% of similarity, more preferably with more than 75% and most preferably with more than 80% by other predictions and given a calculated score (from 0-1 with 1 as highest similarity) considering the sequence repetition and the method score result for the epitope prediction, which is later used in the design of the tandem antigen polypeptide for the therapeutic/prophylactic preparation.

In an alternative embodiment of the present invention, the B-cell binding prediction is done with the entire amino acid sequence of the virus epitope without the preselection of the conserved regions between different virus regions as described in step b). The results are than compared with the conservative regions and filtered. This has the advantage, that overlapping tandem peptides of the combination preparation can contain different sequences matching B-cells thus, also only moderate conserved regions can still produce a good binding to some B-cells and led to an immunization.

According to one embodiment of the current invention, the cytotoxic T lymphocytes (CTL) immunogenic response (CD8) can be predicted with an epitope analysis of the conserved regions, preferably those as identified with means described in b).

Cytotoxic T lymphocytes (CTL) recognize the infected cells by using the MHC class I molecules, which is a sequence with a length of typically 9-10 amino acids, which can bind with certain CTL epitopes. A sequence from a viral epitope can elicit a T-cell CD8 response if it corresponds to an MHC class I molecule which binds to the CTL. The prediction is linear because the binding is based on the sequence of the amino acid sequence. The sequences of the conserved viral epitope as determined in b), can be aligned using a suitable tool or algorithm. Any means known to the skilled person can be used, preferably NetMHCpan 4.1 server from IEDB to predict the potential CTL epitopes. From the whole protein sequence 9-10 amino acid length epitopes that match MHC class I complexes are obtained. Preferably, the threshold (NetMHC threshold, default equal 2 or less) of the peptide rank is 4 or less, more preferably 3 or less, most preferably equal to 2 or less.

In one embodiment of the current invention, the helper T lymphocytes (HTL) immunogenic response (CD4) can be predicted with an epitope analysis of the conserved regions, preferably those as identified with means described in b).

Helper T lymphocytes (HTL) activate other immune cells and recognize an infection by docking to antibodies corresponding to MHC class II epitopes. The prediction is linear because the binding is based on the sequence of the amino acid sequence. The sequences of the conserved epitopes as determined in b), can be aligned using a suitable tool or algorithm. Any means known to the skilled person can be used, preferably NetMHCIIpan and consensus server from IEDB server to predict the potential CTL epitopes by comparing the conservative region with the most common HLA class II alleles (worldwide population, see table 9). From the whole protein sequence of preferably 10-20 amino acids in length, more preferably 12-18 amino acid length epitopes that match MHC class II complexes are obtained. Preferably, the threshold for a weak binding allele (NetMHC threshold, default equal 10 or less) the peptide rank is 14 or less, more preferably 12 or less, most preferably equal to 10 or less and for a strong binding, the binding affinity is preferably equal to two or less. In a preferred embodiment of the current invention, the virus epitope can be prolongated to cover more HTL alleles, the CTL scores are secondary in this case. This is done in order to ensure a broad coverage of the immunizable human population through a broader HTL coverage, aiming for at least 95% of the worldwide population with the combination tandem polypeptide in the spirit of the current invention.

In a particularly preferred embodiment of the present invention, prediction of HTL epitope match and CTL epitope match is performed in parallel with prediction of B-cell epitope match using NetMHC (CTL), NetMHCpan (HTL and CTL), and consensus (HTL) and a final comparison step using MHCPred. Preferably, highly conserved regions are used for prediction, but can be extended to moderately conserved regions if the inclusion of a longer amino acid sequence improves binding results or allows the inclusion of more HTL alleles. Candidates are expanded according to optimize the parallel result score with an optimization algorithm and/or neural network that maximizes binding affinities at all scores (HTL, CTL, and B-cell binding) by including longer sequences of the viral epitope.

According to an especially preferred embodiment of the current invention, the algorithm and/or neural network also optimizes the protein coverage of the viral epitope to bring together sequences from different parts of the virus into one polypeptide. Therefore, it is possible to improve the prediction threshold for HTL match to 5 or less and for CTL match to 1 or less. This has the technical effect of allowing a broader population to be immunized with the same tandem polypeptides that match B-cell, HTL, and CTL epitopes from different conserved parts of the virus epitope (analyzed by comparing different virus variants and mutations), resulting in a strong immunological response and high resistance to mutation.

d) In one embodiment of the invention, the physiological and physicochemical properties of the amino acid sequences are determined and evaluated. A selection of amino acid sequences, preferably those selected with means according to step a-c) are tested for antigenicity with compatibility algorithms. Preferred examples of such algorithms are ANTIGENpro, or preferably VaxiJen, since it was found by the inventors that the results for the amino acid sequences are not correlated with ANTIGENpro. The in-silico antigenicity determination has the advantageous effect to prevent allergic reactions in in vivo studies which would be a major setback in terms of time and finances.

In one embodiment, the preselected results from a-c) are then tested with allergenicity prediction algorithms, preferably AllerTOP, and/or AllergenFP. The In a more preferred embodiment of the invention, booth tools are used, and an amino acid sequence is considered not allergenic if the Tanimoto index is preferably below 0.8, more preferably below 0.7.

In an especially preferred embodiment of the current invention, the physicochemical properties of the amino acid sequences from a-c) are determined. The solubility of the peptides is determined using a program such as SolPro, which can be run with any length of amino acid, the toxicity potential is determined using a program such as ToxinPred, and other physicochemical properties such as hydrophobicity, pl, charge, molecular weight, which are important for the synthesis of these peptides, are determined using any means known to those skilled in the art. In the case of epitopes, it is important to consider the molecular weight, length, pl and charge at pH 5-6 and GRAVY, as it is advantageous to have intermediate weight and polar epitopes at subcutaneous pH (5-6) to avoid precipitation and preferences for hydrophobic peptides (positive GRAVY results). Hydrophobic properties allow absorption by the lipophilic subcutaneous environment while avoiding the formation of salts in the slightly acidic environment.

e) In one embodiment of the current invention, artificial tandem polypeptides are created matching B-cell and T-cells and HLA alleles coverage, using an in-house machine learning (ML) approach to predict the immunogenicity and verifying the results with in vivo feedback. In a preferred embodiment, the ML is thus continuously improved by feedback from in vivo immunogenicity studies such as elispot and ELISA. The immunogenic epitopes predicted based on an in-house ML model, since the binding affinity determined in accordance with the current invention as specified in the previous step b) is not accurate enough in itself to predict whether a polypeptide of the invention is immunogenic or not.

In one embodiment of the current invention, the prediction of the immunogenicity of an antigen polypeptide matching a viral epitope was done using an SVM-based model which was trained with the IEDB database (human binding epitopes tested by Elispot and ICS). Therefore, a trained ML model in accordance with the current invention preferably has an accuracy of at least 50%, more preferably at least 65%, and most preferably more than 70% accuracy in a polypeptide immunogenicity prediction.

In a particularly preferred embodiment, the number of false positives can be reduced compared to standard binding prediction methods by subsequently controlling the results with classical epitope binding affinity calculations, thereby achieving a preferred accuracy of at least 80%, more preferably with at least 90%. Furthermore, in a preferred embodiment, the model considers the physicochemical properties of the polypeptides such as hydrophobicity, pl, charge, molecular weight molecular weight, which are important for the synthesis of these peptides. For polypeptides, it is important to consider the molecular weight, pl and charge at pH 5-6 and GRAVY, since according to the invention, intermediate and polar polypeptides are used at subcutaneous pH (5-6) to avoid precipitation and as well as preferential weighting for hydrophobic peptides (positive GRAVY results). This has the technical effect of better simulating in vivo conditions and therefore more appropriate antigen peptide selection compared to binding affinity predictions alone, resulting in better immunization and shorter development times as less in vivo testing is required.

In a particularly preferred embodiment, calculated values related to an antigen polypeptide are considered for the ML model training, such as immunogenicity, using means known to a person skilled in the arts, for example IEDB, the TAP proteosome using a program such as NetCTLpan, and the cleavage sites using a program, such as NetChop.

In one embodiment of the current invention, the trained ML model correlation data between inputs and outputs can be quantified and is obtained in proportions of 0 to |1|. Therefore, it is possible to quantify the parameters which are particularly influential in the polypeptide selection, thus allowing for further, step by step improvement of the data.

One example of such a trained ML model and the correlation between input and output data is shown in FIG. 15, which does represent a fraction of the current invention and is not limited by it. In this particular embodiment of the current invention, the BA-rank result from NetMHCpan is the highest correlation result compared with the rest of the inputs.

It is therefore an outstanding achievement of the inventors to have found that additional factors can be considered using machine learning (ML) to predict immunogenicity in silico. The antigen polypeptides determined in the previous steps must be processed in the organism and presented to the T-cell receptors. This has the technical effect that the model generated by this approach resembles much more in vivo conditions, where chemistry, such as protonation reactions, precipitations, cleavage of the peptides occur, and thus pure binding affinity predictions correlate poorly with in vivo tests. Therefore, a much better predictions leads to better, safer, and quicker candidate selection and the feedback von vivo studies can directly be fed back into the ML algorithm to further improve the results.

In a particular embodiment of the current invention related to the ML model shown in FIG. 15, input and output parameters include MHCflurry inputs (mhcflurry_affinity percentile, mhcflurry_processing_score and mhcflurry presentation_score), number of amino acid cleavage points (Number), Mass-Spectrometry eluted ligands (EL) peptides (EL Rank) and corresponding quantitative binding affinity (BA) predictions (BA Rank), the physicochemical properties in the model are the isoelectric point (pl), net charge of the peptide at pH=5-6 (Charge), grand average of hydropathy (GRAVY), IEDB immunogenicity (Score), transporter associated with antigen processing (TAP) proteosome information (TAP), cleavage probability at the N-terminus (Cle), the combined score of Score, TAP and Cle (comb_score), and ranking of all (Rank_AL). Parameters especially preferred in accordance with the current invention which have a high correlation between input and output include EL_Rank, Ba_Rank, Rank_AL, mhcflurry_presentation_score, Comb_score.

In an alternative embodiment of the current invention, inputs may be excluded from the machine learning algorithm to improve the results. It is a discovery of the inventors, that charge input is the less influential in the database decision. In some embodiments of the invention entries were removed after checking the correlation between them and the output, as for example the Rank_AL (is very similar to comb_score), EL_Rank, mhcflurry_binding_affinity and Comb_score due to its similaty to the mhcflurry_presentation_score and its limitation in the prediction tool. This reduces the variables needed for training and reduces redundancies, thus allowing the ML algorithm to optimize more relevant parameters and to a more sophisticated ML model.

In a preferred embodiment, the amino acid sequences, also referred to as artificial tandem polypeptides, as determined in step a-d) are weighted by a weighting algorithm/neural network also referred to as ML model, which weights first HLA alleles coverage, T-cell and B-cell binding affinity scores and optimized across the three, while also considering the protein coverage and optimizing the distribution of the combination peptides across different proteins of the viral epitope, for example a viral nucleocapsid, or for example a viral spike protein. This has the advantage of a broader immunization spectrum against different mutants and/or viral variants and a more robust immune response and thus a better immunization against the specific viral strains.

In an especially preferred embodiment of the current invention, a pool of amino acid sequences is therefore created, comprising of at least one, more particularly at least two and most particularly up to 12 different synthetic peptide complexes (antigen polypeptides) corresponding to HLA antigen peptides matching viral epitopes, for immunization against a virus while avoiding infection-enhancing antibodies. Any amino acid sequences with allergenicity or antigenicity concerns, as well as unsuited physicochemical properties, such as poor solubility, too high molar mass, chemical instability, mismatched GRAVY value, physiological pH combability or toxicological properties. In peptides the instability, aliphatic index and half-life is not as important as for proteins, therefore these properties only have a low weight in the overall optimization.

In some embodiments of the current invention, the following thresholds have been introduced on the preselection of amino acids for the weighting, explicitly on allergenicity, solubility, human proteome alignment and toxicity. The use of such cutoff points represents a discovery by the inventors that has the technical effect of significantly improving the ML model used and each value is a discovery of its own by the inventors. The following cutoff points significantly improve the model training and are the result of a difficult optimization process.

In a preferred embodiment of the current invention, the following thresholds have been introduced on the preselection of amino acids for the weighting, explicitly on allergenicity, solubility, human proteome alignment and toxicity. Amino acid sequences where excluded which had antigenic predictions with a value below 0.4, toxic epitopes, peptides with higher similarity than 35% to human proteome and non-soluble peptides with a solubility value below 0.5. Only amino acid sequences with HTL matching below 5 and sequences matching alleles with CTL matching below 1 were included. The highest weighted factor is the binding affinity in the B-cell prediction (factor 1), then the coverage results for HTL 5 (factor 0.7) and number of alleles in CTL (factor 0.7). These preferred weighting factors represent a discovery by the inventors which improve the results of the in-silico immunogenicity prediction drastically which leads to a better modelling of immune response in a subject.

In an especially preferred embodiment of the current invention, amino acid sequences in the pool can be exchanged, if mutations alter the conservative regions of the virus epitope. In an especially preferred embodiment of the invention, the polypeptide pool is created in a redundant way, such as multiple overlapping polypeptides containing the same HLA peptides in different compositions while covering a broad selection of the virus proteins, in such a way that a single amino acid substitution in a new viral variant doesn't affect the combination preparation.

In a preferred embodiment of the invention the algorithm also considers the HLA coverage of the antigen polypeptides the most common alleles to cover preferably at more than 95%, more preferably more than 98% of the global population. Preferably, the most common HLA A and B alleles are chosen from at least 20 MHC Class I alleles from table 9, more preferably more than 24, most preferably at least 27 MHC Class I alleles from table 9. Therefore, a single combination preparation can be used to immunize nearly the worldwide human population. In an especially preferable embodiment, a single tandem antigen polypeptide matching HLA A and B alleles covers preferably more than 95% of the global population.

In an embodiment of the invention the HLA coverage value is automatically calculated, in some cases amino acid sequences with lower T-cell and/or B-cell scores can be included to improve the robustness and coverage of the combination preparation.

### Process for the Preparation of a Pharmaceutical Composition

The present invention further comprises a method for preparing a pharmaceutical composition or formulation according to the invention, said method comprising the following steps:
(a) determining at least one HLA antigen peptides corresponding to MHC class I complexes and/or at least one antigen polypeptide exposed on the cell surface of cells of the viral infectious disease(s) of the patient or group of patients to be treated having the same haplotype, using the determination method according to the invention as described above;
(b) synthesizing the HLA antigen peptide(s) determined in step (a) according to the MHC class I complexes and/or at least one antigen polypeptide, wherein the definition of each HLA antigen peptide is the same as defined above; and
(c) preparing the pharmaceutical composition according to the invention comprising at least the HLA antigen peptides synthesized in step (b) corresponding to the MHC class I complexes and/or at least one antigen polypeptide and an adjuvant as defined herein.

Also encompassed by the invention is a pharmaceutical composition comprising an antigen peptide as defined herein and a pharmaceutically acceptable excipient.

In a more preferred embodiment, the invention comprises a method for preparing a pharmaceutical composition comprising the following steps:
(a) determining at least one, preferably at least two, more preferably at least three, particularly preferably at least five, most preferably at least 7, alternatively up to twelve antigen polypeptide corresponding to MHC class I complexes matching at least two viral epitopes, preferably of different viral diseases according to the method as disclosed herein, which are matching preferably at least 95%, more preferably at least 98% of the most common HLA alleles distributed in the worldwide population;
(b) synthesizing the antigen polypeptide(s) corresponding to MHC class | complexes determined in step (a);
(c) preparing the pharmaceutical composition according to the invention comprising at least one antigen polypeptide(s) corresponding to MHC class I complexes matching at least one viral epitope, preferably in a suitable pharmaceutical formulation.

The method allows simultaneous treatment or prophylaxis of at least two viral infectious diseases using the same amount of antigen peptides as a single vaccination, reducing potential side effects while providing broader and stronger immunization.

The invention also relates to an HLA antigen peptide and/or antigen polypeptide of the invention for use in the preparation of a formulation (such as, without limitation, a pharmaceutical formulation as further described herein) for the treatment of viral infectious disease, either in vitro (e.g. in an invitro or cellular detection method) or in vivo (such as in a unicellular or multicellular organism and more particularly in a mammal and more particularly in a human being, such as a human being at risk of contracting or suffering from viral infectious disease according to the current inventions).

### Detailed Description and Definitions

In the present description and claims, the following terms are defined as follows:

In the context of the present invention, the features of the invention defined by “comprising” are intended to include the more limited description of “consisting of” or “consisting essentially of” the same features of the present invention.

The term “and/or” is used to specifically disclose the two features or components together or separately. Therefore, the term “and/or” as used, for example, in the phrase “I and/or II” in the present disclosure includes “I and II”, “I or II”, “I” and “II”.

For purposes of the present invention, an HLA antigen peptide is one if it is a at least one viral infectious disease exclusive HLA antigen peptide.

An immunogenic HLA antigen peptide is also referred to herein as an “epitope.”

A pharmaceutical composition is herein to be understood as a so-called informatic that can be administered to a person or a group of persons having at least one identical HLA allele and that contains the combination of HLA antigen peptides according to the invention in the concentration disclosed herein, wherein an HLA antigen peptide represents an information carrier. This means that a sequence-specific activation of the immune system, in particular of T cells (by HLA antigen peptides corresponding to MHC class I complexes) and/or B cells (by HLA antigen peptides corresponding to MHC class II complexes) is induced by the arrangement of amino acids in the amino acid sequence of the HLA antigen peptides (“code”).

The primary objective of the present invention is to induce targeted activation and training of the immune system, in particular of T cells and B cells against viral infectious diseases. For this reason, the pharmaceutical composition according to the invention is preferably applied intradermally. This has the particular advantage that the immune system or the T cells, which recognize the applied HLA antigen peptides, process this information applied in the form of antigen peptides and consequently specifically recognize and lyse body cells affected by viruses which present these HLA antigen peptides on their surface. At least one HLA antigen peptide within the scope of the present invention is particularly formulated for intradermal administration. The expression/term “composition” therefore refers to the provision of at least one HLA antigen peptide and an adjuvant in a pharmaceutical formulation that allows good applicability and includes solutions, in particular injection solutions, concentrates for the preparation of injection preparations, or powders for the preparation of injection preparations.

As used herein, the term “class I HLA antigen peptide (corresponding to MHC complexes)” means a peptide sequence that is bound to or immunogenic for the class I MHC complex (in humans, the HLA complex). The class I HLA protein complex is used for antigen presentation on the cell surface and comprises a heavy chain with 3 domains (α1, α2, and α3) and the β2-microglobulin (β2M).

As such, the polypeptides and pharmaceutical compositions of the present invention (as defined herein) may be used for use in the prevention and treatment of a virus (also referred to herein as “viral infectious disease”, “infectious disease”). In general, the “infectious diseases of the invention” may be defined as diseases and disorders that can be appropriately prevented and/or treated by appropriate administration of either an HLA antigen peptide or a pharmaceutical composition of the invention (and, more particularly, a pharmaceutically effective amount thereof) to a subject (i.e., a person having the disease or disorder, or at least one symptom thereof, and/or who is at risk of acquiring or developing such disease or disorder).

For purposes of the present invention, a “peptide sequence” (e.g., an HLA antigen peptide) having a “native sequence” comprises a peptide sequence having the same (i.e., unmodified) amino acid sequence as a naturally occurring peptide sequence in the subject. Such a peptide sequence with a “native sequence” can be isolated from nature or produced recombinantly or synthetically. In particular, the term peptide sequence having a “native sequence” includes naturally occurring truncated or secreted forms of the peptide sequence (e.g., an extracellular domain sequence), naturally occurring variants (e.g., alternatively spliced forms), and naturally occurring allelic variants of the peptide sequence.

In this context, the pharmaceutical composition is preferably applied subcutaneously (“under the skin”), or intradermally (“into the skin”), or intramuscularly (“into the muscle”). The application may be intramuscular as an injection into the thigh (vastus lateralis muscle), preferably into the upper arm (deltoid muscle).

As further described herein, the amino acid sequences used in the invention are single variable HLA antigen peptide domains (“HLAs” or “HLA complex”). A single variable HLA antigen peptide domain is (as further defined herein) a region within the amino acid sequence of a protein that can be distinguished from its surrounding sequence based on defined characteristics.

Amino acid sequences, or regions within the amino acid sequence of a protein of the invention, that are HLAs are also referred to herein as “HLAs of the invention.” Some preferred examples of single variable HLA antigen peptide domains suitable for use in the invention are apparent from the further description herein, and particularly include HLA-A and/or HLA-B antigen peptides corresponding to MHC class I complexes.

The following terms are used to describe sequence relationships between two or more amino acid sequences or polypeptide sequences: “reference sequence,” “amino acid exchange,” “sequence identity,” “percentage of sequence identity,” and “substantial identity.”

In the context of the present invention, the term “coronavirus” is used preferably to describe a orthocoronavirinae, more preferably to describe betacoronaviruses, particularly preferably to describe a sarbecovirus, most preferably SARS-COV-2, especially a wild-type (WT) or mutant SARS-COV-2, such as the Alpha (B.1.1.7), or Beta (B.1.351), or Gamma (P. 1), or Delta (B.1.617.2) and/or Omicron variant (B.1.1.529).

In the context of the present invention, an infectious viral disease can be any disease caused by a virus (pathogen or infectious agent) transmitted between subjects. The subjects are preferably mammals, more preferably humans. The subjects can alternatively be any host suitably for transmitting the viral disease to a subject, preferably where the subject is a mammal, more preferably where the subject is a human.

In the context of the present invention, a “virus” can be any DNA virus and/or RNA virus and/or retrovirus, for example but not limited to Coronaviruses, such as SARS-COV-2 (a single-stranded RNA virus), and/or Orthomyxoviridae, a virus family containing Alphainfluenzavirus, Betainfluenzavirus, Gammainfluenzavirus, Deltainfluenzavirus, Isavirus, Thogotovirus, and Quaranjavirus (negative-sense RNA viruses) and/or poliovirus (from the family of Picornaviridae with single-stranded positive-sense RNA), and/or Rotavirus (from the family of Reoviridae with double stranded RNA viruses), and/or tick-borne encephalitis virus (TBEV, from the family of Flaviviruses with a positive-strand RNA), and/or human papillomavirus (HPV, from the Papillomaviridae family of DNA viruses).

In the context of the present invention, the term “amino acid exchange” refers to the exchange of one amino acid for another amino acid within the amino acid sequence of the HLA antigen peptide to be synthesized relative to the wild-type of that HLA antigen peptide (i.e., native HLA antigen peptide). “Sequence identity,” “percentage of sequence identity,” or identity or similarity with respect to said amino acid sequence is defined herein as the percentage of amino acid residues in the amino acid sequence of the polypeptide that is identical (i.e., same residue) or similar (i.e., amino acid residue from the same group based on common side chain characteristics, see below) to the amino acid sequence of the wild type.

According to a preferred embodiment of the present invention, the amino acid substitution for the HLA antigen peptide corresponding to the class I and/or class II MHC complexes comprises at least one substitution at any position within the amino acid sequence relative to the wild type of that HLA peptide.

For the purpose of comparing two or more amino acid sequences, the percentage of “sequence identity” between a first amino acid sequence and a second amino acid sequence may be calculated or determined by dividing the number of amino acids in the first amino acid sequence that are identical to amino acids at corresponding positions in the second amino acid sequence, by [the total number of amino acids in the first amino acid sequence] and multiplying by [100%], wherein each deletion, insertion, substitution or addition of an amino acid in the second amino acid sequence-compared to the first amino acid sequence—is considered as a difference to a single amino acid (position).

The term “disease” refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

In the present context, the term “treatment” or “treating” relates to the management and care of a subject or an individual for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The amino acids used herein are abbreviated according to the generally accepted 15 one-letter code of the IUPAC Nomenclature Commission. Where two amino acids are separated by a hyphen (/), this indicates that at a specific amino acid position in the amino acid sequence concerned, the wild-type amino acid (left side of the hyphen) has been replaced by another amino acid (right side of the hyphen). In the present invention, the amino acids (IUPAC one-letter code) S, T, C, N, Q, Y are considered hydrophilic acids; the amino acids (IUPAC one-letter code) A, F, G, I, L, M, P, V, W are considered hydrophobic acids; the amino acids (IUPAC one-letter code) D, E, are considered acidic; the amino acids (IUPAC one-letter code) R, K, H are considered basic acids.

The terms “immunization” or “vaccination” describe the process of administering an antigen to an individual with the purpose of inducing an immune response, for example, for therapeutic or prophylactic reasons.

The term “individual” (also referred to herein as “person” or “subject” or “patient”) is used interchangeably with the term “subject” to mean any mammal (e.g. mouse, rat, rabbit, dog, cat, cattle, swine, sheep, horse or primate) that is being treated for an abnormal physiological condition or has been diagnosed with a disease caused by a virus.

The terms “individual” and “subject” as used in the present invention include mammals, such as a rodent, a cloven-hoofed animal, an odd-toed ungulate, or a primate. In particularly preferred embodiments, the subject is a human. Unless otherwise stated, the terms “individual” and “subject” do not denote a particular age, and thus encompass adults, elderlies, children, and newborns. In some embodiments, the term “subject” includes humans of age of at least 50, at least 55, at least 60, at least 65, at least 70, or older. In some embodiments, the term “subject” includes humans of age of at least 65, such as 65 to 80, 65 to 75, or 65 to 70.

For the purposes of the invention, the totality of all HLA antigen peptides presented on the cell surface via MHC molecules is referred to as the (HLA) ligandome. It is believed that more than 105 HLA molecules are expressed on the cell surface and the number of identical HLA proteins presented can vary from a few to up to 10,000 copies per cell. Consequently, approximately 10,000 different HLA proteins are presented on a cell in varying proportions.

The ligandome is influenced by various physiological, intrinsic as well as pathological factors such as cell type or tissue type, infection or transformation of the cell, or simply the current state of the cell, which depends on nutrient situation or external stress factors, resulting in changes in the HLA proteins presented.

At the beginning of the analysis of the HLA ligandome, for example, Edman degradation can be used to gain initial insights into the presented peptides. On the one hand, it is possible in this way to determine the general peptide motif of an allele via pool sequencing, and on the other hand, individual peptide sequences can already be determined via the analysis of individual reversed phase high performance liquid chromatography (RP-HPLC) fractions.

Alternatively, or complementarily, the analysis of the ligandome can be performed by using modern mass spectrometers in proteomics, with which it is possible to unambiguously determine the sequences of many individual ligands. Two methods are used for the ionization of the peptides or proteins required for this purpose: Electrospray ionization (ESI) and matrix-assisted laser desorption/ionization (MALDI). In ESI, coupling with an RP-HPLC system is common. However, as mass spectrometers have become more sensitive, capillary electrophoresis (CE) has also been used as an analytical separation method.

In order to achieve a higher sample throughput and increased sensitivity in ligandome analysis, the so-called UHPLC systems (ultra-high performance liquid chromatography) can be used. These HPLC systems use only 2.2 to 1.7 μm diameter materials as packing material for separation columns, resulting in improved speed, efficiency, and chromatographic separation.

In ESI mass spectrometry, the direct coupling of HPLC and ESI interface allows on-line separation of the sample, which, in combination with an autosampler, enables a fully automated measurement procedure. Because of the continuous solvent flow from the HPLC, samples can be measured in a relatively short time. ESI mass spectrometry uses a wide range of instruments for analysis such as quadrupole time-of-flight mass spectrometers, linear quadrupole ion traps, triple quadrupoles, or ion trap-orbitrap hybrid systems. This advantageously allows the identification of hundreds of HLA peptides in one measurement.

IFNγ ELISpot analysis (for protocol see A. Lalvani, R. Brookes, S. Hambleton, W. J. Britton, A. V. S. Hill, A. J. McMichael, J Exp Med 1997; 186 (6): 859-865) was performed ex vivo (without further in vitro culturing for expansion) using PBMCs depleted of CD4+ and enriched for CD8+ T cells (CD8+ effectors), or depleted of CD8+ and enriched for CD4+ T cells (CD4+ effectors). Tests were performed in duplicate and with a positive and negative control, Multiscreen filter plates (Merck Millipore) pre-coated with IFNγ-specific antibodies (ELISpotPro kit, Mabtech). 267.000 Cells were used per plate. Plates were scanned using an AID Classic Robot ELISPOT Reader and analyzed by AID ELISPOT 7.0 software (AID Autoimmun Diagnostika). Antigen Peptides (except negative and positive control group) were done in duplicates, spot counts were taken as mean values of each duplicate.

To provide antigen peptides, in principle, synthetic or isolated HLA antigen peptides derived from the cumulative ranking can be used for the preparation of the (medicament) formulations of the present invention and for the preparation of the pharmaceutical composition (as so-called informaticum).

The term “active ingredient enhancer/adjuvant” refers to an excipient that triggers and/or enhances the effect of the HLA peptides in the first place. In principle, all commonly used adjuvants known to the skilled person are suitable for the preparation of a formulation according to the invention.

### Advantages and Description of the Process

However, it is convenient to use synthetic HLA peptides. Processes for the synthetic preparation of peptides are known to those skilled in the art. Examples of such production methods are the Merrifield solid-phase peptide synthesis, the Bailey peptide synthesis, and the N-carboxylic anhydride method.

Machine Learning, also referred to as neural network, or machine learning model, for the purposes of this invention, means any algorithm that can use machine learning to build a model based on sample data, referred to as training data, to make predictions or decisions. This algorithm does not have to be explicitly programmed for these circumstances but builds its model exclusively on the training data set.

### BRIEF DESCRIPTION OF THE DRAWINGS

Herein shows:

FIG. 1: Schematic overview of the immunization scheme for CoV-WT-2

Four groups of wild-type mice, one reference group (no vac.), two groups (2,3) injected with a pharmaceutical composition in accordance with the present invention (SEQ IDs SEQ ID NOS: 38-49) and one group injected with an mRNA vaccine.

FIG. 2 Comparison of Group 1-4 INFγ CD8+ ICS

Intracellular cytokine staining (ICS) with ELISpot analysis, CD8 T-cell responses, activity IFNγ+CD8+. *-compared to no vac. reference group. Horizontal lines indicate median values.

FIG. 3 Comparison of Group 1-4 INFγ CD8+IL-2+ICS

Intracellular cytokine staining (ICS) with ELISpot analysis, CD8 T-cell responses, activity IFNγ+ TNFα+IL-2+CD8+. (total) *-compared to no vac. reference group. Horizontal lines indicate median values.

FIG. 4 Comparison of Group 1-4 INFγ CD4+ICS

Intracellular cytokine staining (ICS) with ELISpot analysis, CD8 T-cell responses, activity IFNγ+CD4+*-compared to no vac. reference group. Horizontal lines indicate median values.

FIG. 5 Single peptide-specific CD8 T-cell ICS (Pep X)

Pep X, Intracellular cytokine staining (ICS), Single peptide-specific CD8 T-cell responses corresponding to peptides from SEQ ID NOs: 38-49, OVA: unrelated peptide (*-compared to no vac. reference group)

FIG. 6 Single peptide-specific CD8 T-cell ICS (Pep C)

Pep C, Intracellular cytokine staining (ICS), Single peptide-specific CD8 T-cell responses corresponding to peptides from SEQ ID NOs: 38-49, OVA: unrelated peptide (*-compared to no vac. reference group)

FIG. 7: Schematic overview of the immunization scheme for CoV-WT-2

Three groups of wild-type mice, one reference group (no vac.), one group injected with a pharmaceutical composition (SEQ ID NOs: 43, 44, 46, 47, 50-58) in accordance with the present invention and one group injected with an mRNA vaccine.

FIG. 8 Single peptide-specific CD4 T-cell ICS

Intracellular cytokine staining (ICS), Single peptide-specific CD4 T-cell responses corresponding to peptides from SEQ ID NOs: 38-49, OVA: unrelated peptide (*-compared to no vac. reference group)

FIG. 9 Single peptide-specific CD8 T-cell ICS

Intracellular cytokine staining (ICS), Single peptide-specific CD4 T-cell responses corresponding to peptides from SEQ ID NOs: 43, 44, 46, 47, 50-58, OVA: unrelated peptide (*-compared to no vac. reference group)

FIG. 10 Single peptide-specific CD8 T-cell Human EliSpot

Intracellular cytokine staining (ICS), cells/100000 and INFγ activation, single peptide-specific CD8 T-cell responses corresponding to peptides from SEQ ID NOs: 43, 44, 46, 47, 50-58, neg=negative control.

FIG. 11 Single peptide-specific CD8 T-cell Human EliSpot

Intracellular cytokine staining (ICS), Total INFγ Activity, single peptide-specific CD8 T-cell responses corresponding to peptides from SEQ ID NOs: 43, 44, 46, 47, 50-58, neg=negative control.

FIG. 12: INFγ CD8+EliSpot responses of human PBMCs

ELISpot well plates corresponding to FIG. 10/11 results of peptide activation with peptides from SEQ ID NOs: 43, 44, 46, 47, 50-58, neg=negative control, pos=positive control.

FIG. 13: 3D Structure Prediction for antigen Polypeptide SEQ-ID NO.: 89 and 90 Three-dimensional peptide folding prediction (galaxy Refinement) for the synthetic antigen polypeptides SEQ-ID NO.: 89 (right) and SEQ-ID NO.: 90 (left) used for binding pocket prediction.

FIG. 14: Binding affinity prediction and pocket analysis in 3D model

The Binding affinity of the synthetic antigen polypeptide SEQ-ID NO.: 89 (left) and 90 (right) with the crystallized 6EIX ligand-receptor with polar interaction residues between ligand and receptor.

FIG. 15: Trained machine learning (ML) model with input/output correlation values

IEDB training data correlation plot between different entries (binding affinity results, TAP proteosome, cleavage, physicochemical properties, and immunogenicity (Qualitative_measure) in vitro tested via Elispot and ICS. The trained model correlation data between inputs and outputs (Qualitative_measure entry) is obtained in proportions of 0 to |1|.

### EXAMPLES

With reference to the following figures and embodiments, the present invention will be explained in more detail without limiting the invention thereto.

The following tables list HLA antigen peptides which are one embodiment of the current invention related to coronaviruses, all of which have been tested and are immunogenic. SEQ ID NOs: 38-58, more preferably 39-58 and/or 80-90 list some preferred, but not limiting, examples of amino acid sequences of antigen peptides of the invention for the use as vaccination against SARS-COV-2, each of which is another embodiment of the present invention.

### Tables

TABLE 1
SARS-CoV-2 Spike Protein

| | Subtype | | | |
|---|---|---|---|---|
| | Specific | | | |
| | Antigen- | HLA | Reaction | |
| HLA Isotype | Variant | Peptide | Value | Target Protein |
| HLA-A Antigen peptide | HLA-A0201 | LLFNK | 25.41 | Chain A, SARS-CoV-2 spike |
| (SEQ ID NO.: 1) | | VTLA | | glycoprotein (821-846) |
| HLA-B Antigen peptide | HLA-B0801 | FIKQY | 95.44 | Chain A, SARS-CoV-2 spike |
| (SEQ ID NO.: 2) | | GDCL | | glycoprotein (821-846) |
| HLA-A Antigen peptide | HLA-A0301 | TLADA | 198.58 | Chain A, SARS-CoV-2 spike |
| (SEQ ID NO.: 3) | | GFIK | | glycoprotein (821-846) |
| HLA-A Antigen peptide | HLA-A0301 | KCYGV | 152.62 | Chain A, SARS-CoV-2 spike |
| (SEQ ID NO.: 4) | | SPTK | | glycoprotein (378-396) |
| HLA-A Antigen peptide | HLA-A2402 | CYGVS | 1071.56 | Chain A, SARS-CoV-2 spike |
| (SEQ ID NO.: 5) | | PTKL | | glycoprotein (378-396) |
| HLA-A Antigen peptide | HLA-A0301 | RLFRK | 5.86 | Chain A, SARS-CoV-2 spike |
| (SEQ ID NO.: 6) | | SNLK | | glycoprotein (441-466) |
| HLA-A Antigen peptide | HLA-A2402 | NYNYL | 28.87 | Chain A, SARS-CoV-2 spike |
| (SEQ ID NO.: 7) | | YRLF | | glycoprotein (441-466) |
| HLA-B Antigen peptide | HLA-B2705/ | YRLFR | 66.99/ | Chain A, SARS-CoV-2 spike |
| (SEQ ID NO.: 8) | HLA-B0801 | KSNL | 317.1 | glycoprotein (441-466) |
| HLA-A Antigen peptide | HLA-A2402 | PYRVV | 152.38 | Chain A, SARS-CoV-2 spike |
| (SEQ ID NO.: 9) | | VLSF | | glycoprotein (497-521) |
| HLA-B Antigen peptide | HLA-B3901/ | YQPYR | 79.44/ | Chain A, SARS-CoV-2 spike |
| (SEQ ID NO.: 10) | HLA-B1501 | VVVL | 131.99 | glycoprotein (497-521) |
| HLA-B Antigen peptide | HLA-B5801 | RVVVL | 290.5 | Chain A, SARS-CoV-2 spike |
| (SEQ ID NO.: 11) | | SFEL | | glycoprotein (497-521) |
| HLA-B Antigen peptide | HLA-B2705 | YRFNG | 435.93 | Chain A, SARS-CoV-2 spike |
| (SEQ ID NO.: 12) | | IGVT | | glycoprotein (902-918) |

TABLE 2
SARS-CoV-2 Nucleocapsid Protein

| | Subtype | | | |
|---|---|---|---|---|
| | Specific | | | |
| | Antigen- | | Reaction | |
| HLA Isotype | Variant | HLA Peptide | Value | Target Protein |
| HLA-B Antigen peptide | HLA- | FPRGQGVPI | 3.82/ | Nucleocapsid |
| (SEQ ID NO.: 13) | B0702/ | | 1037.41 | phosphoprotein [Bat |
| | HLA- | | | coronavirus HKU9-3] (80-88) |
| | B3901 | | | |
| HLA-B Antigen peptide | HLA- | KPRQKRTAT | 4.42/ | Chain A, SARS-CoV-2 |
| (SEQ ID NO.: 14) | B0702/ | | 364.72 | nucleocapsid protein (7-37) |
| | HLA- | | | |
| | B0801 | | | |
| HLA-B Antigen peptide | HLA- | KAYNVTQAF | 12.51/ | Chain A, SARS-CoV-2 |
| (SEQ ID NO.: 15) | B5801/ | | 40.35 | nucleocapsid protein (7-37) |
| | HLA- | | | |
| | B1501 | | | |
| HLA-A Antigen peptide | HLA- | GMSRIGMEV | 50.61 | nucleocapsid protein N |
| (SEQ ID NO.: 16) | A0201 | | | [SARS coronavirus WH20] |
| | | | | (315-331) |
| HLA-B Antigen peptide | HLA- | MEVTPSGTW | 1256.27 | nucleocapsid protein N |
| (SEQ ID NO.: 17) | B5801 | | | [SARS coronavirus WH20] |
| | | | | (315-331) |
| HLA-A Antigen peptide | HLA- | ASAFFGMSR | 292.41 | nucleocapsid protein N |
| (SEQ ID NO.: 18) | A0301 | | | [SARS coronavirus WH20] |
| HLA-A Antigen peptide | HLA- | FAPSASAFF | 422.31/ | nucleocapsid protein N |
| (SEQ ID NO.: 19) | A2402/ | | 349.57 | [SARS coronavirus WH20] |
| | HLA- | | | |
| | A2601 | | | |
| HLA-B Antigen peptide | HLA- | AQFAPSASA | 62.45 | nucleocapsid protein N |
| (SEQ ID NO.: 20) | B1501 | | | [SARS coronavirus WH20] |

TABLE 3
SARS-CoV-2 Envelope Protein

| | Subtype | | | |
|---|---|---|---|---|
| | Specific | | | |
| | Antigen- | | Reaction | |
| HLA Isotype | Variant | HLA Peptide | Value | Target Protein |
| HLA-A Antigen | HLA-A0101 | VSLVKPSFY | 1007.63 | envelope protein |
| peptide (SEQ ID | | | | [SARS-CoV-2] (49- |
| NO.: 21) | | | | 75) |
| HLA-A Antigen | HLA-A0201 | SLVKPSFYV | 9.11 | envelope protein |
| peptide (SEQ ID | | | | [SARS-CoV-2] (49- |
| NO.: 22) | | | | 75) |
| HLA-A Antigen | HLA-A0301 | SFYVYSRVK | 646.15 | envelope protein |
| peptide (SEQ ID | | | | [SARS-CoV-2] (49- |
| NO.: 23) | | | | 75) |
| HLA-B Antigen | HLA-B1501 | LVKPSFYVY | 14.11 | envelope protein |
| peptide (SEQ ID | | | | [SARS-CoV-2] (49- |
| NO.: 24) | | | | 75) |
| HLA-B Antigen | HLA-B5801 | NSSRVPDLL | 311.64 | envelope protein |
| peptide (SEQ ID | | | | [SARS-CoV-2] (49- |
| NO.: 25) | | | | 75) |
| HLA-B Antigen | HLA-B0801 | YVYSRVKNL | 250.69 | envelope protein |
| peptide (SEQ ID | | | | [SARS-CoV-2] (49- |
| NO.: 26) | | | | 75) |

TABLE 4
SARS-CoV-2 Surface Protein

| | Subtype | | | |
|---|---|---|---|---|
| | Specific | | | |
| | Antigen- | HLA | Reaction | |
| HLA Isotype | Variant | Peptide | Value | Target Protein |
| HLA-A Antigen peptide | HLA- | VLNDILSR | 33.57 | Chain A, Surface |
| (SEQ ID NO.: 27) | A0201 | L | | Glycoprotein [SARS-CoV- |
| | | | | 2] (972-988) |
| HLA-B Antigen peptide | HLA- | ISSVLNDIL | 518.51 | Chain A, Surface |
| (SEQ ID NO.: 28) | B5801 | | | Glycoprotein [SARS-CoV- |
| | | | | 2] (972-988) |

TABLE 5
SARS-CoV-2 ORF1ab Protein

| | Subtype | | | |
|---|---|---|---|---|
| | Specific | | | |
| | Antigen- | | Reaction | |
| HLA Isotype | Variant | HLA Peptide | Value | Target Protein |
| HLA-A Antigen peptide | HLA- | FSASTSAFV | 1178 | ORF1ab polyprotein |
| (SEQ ID NO.: 29) | A0101 | | | (SARS-CoV-2] (474- |
| | | | | 488) |
| HLA-A Antigen peptide | HLA- | ILASFSAST | 85.19 | ORF1ab polyprotein |
| (SEQ ID NO.: 30) | A0201 | | | (SARS-CoV-2] (474- |
| | | | | 488) |
| HLA-A Antigen peptide | HLA- | SFSASTSAF | 695.27/? | ORF1ab polyprotein |
| (SEQ ID NO.: 31) | A2402/ | | | (SARS-CoV-2] (474- |
| | HLA- | | | 488) |
| | B1501 | | | |
| HLA-B Antigen peptide | HLA- | AARVVRSIF | 221 | ORF1ab polyprotein |
| (SEQ ID NO.: 32) | B0702 | | | (SARS-CoV-2] (535- |
| | | | | 549) |
| HLA-A Antigen peptide | HLA- | SLPFGWLIV | 219.12 | ORF1ab polyprotein |
| (SEQ ID NO.: 33) | A0201 | | | (SARS-CoV-2] |
| HLA-A Antigen peptide | HLA- | IQASLPFGW | 450.63 | ORF1ab polyprotein |
| (SEQ ID NO.: 34) | A2402 | | | (SARS-CoV-2] |
| HLA-B Antigen peptide | HLA- | ASLPFGWLI | 57.16 | ORF1ab polyprotein |
| (SEQ ID NO.: 35) | B5801 | | | (SARS-CoV-2] |
| HLA-A Antigen peptide | HLA- | RIAGHHLGR | 102.11 | ORF1ab polyprotein |
| (SEQ ID NO.: 36) | A0301 | | | (SARS-CoV-2] |
| HLA-B Antigen peptide | HLA- | HLRIAGHHL | 647.29/ | ORF1ab polyprotein |
| (SEQ ID NO.: 37) | B0702/ | | 494.02 | (SARS-CoV-2] |
| | HLA- | | | |
| | B0801 | | | |

TABLE 6
SARS-CoV-2 specific HLA antigen peptides

| HLA antigen peptide | Identification | Target Protein | Matching |
|---|---|---|---|
| related to (Pep NO) | NO. | SARS-CoV-2 | Epitope |
| AEIRASANL (21) | SEQ-ID NO.: 59 | S2 | B cell, HTL |
| ASANLAATK (21) | SEQ-ID NO.: 60 | S2 | B cell, HTL |
| AYRFNGIGV (7) | SEQ-ID NO.: 61 | S2 | CTL |
| DCLGDIAAR (14) | SEQ-ID NO.: 62 | S2 | B cell |
| DGYFKIYSK (17) | SEQ-ID NO.: 63 | S1 | CTL |
| DILSRLDKV (10) | SEQ-ID NO.: 64 | S2 | CTL |
| DLSPRWYFY (20) | SEQ-ID NO.: 65 | NP | HTL, CTL |
| EYVSQPFLM (13) | SEQ-ID NO.: 66 | S1 | HTL |
| FPRGQGVPI (4, 18) | SEQ-ID NO.: 67 | NP | B cell |
| FQPTNGVGY (6) | SEQ-ID NO.: 68 | S2 | HTL, CTL |
| GYFKIYSKH (17) | SEQ-ID NO.: 69 | S1 | CTL |
| GYQPYRVVV (6) | SEQ-ID NO.: 70 | S2 | HTL, CTL |
| IGVTQNVLY (7) | SEQ-ID NO.: 71 | S2 | CTL |
| KDLSPRWYF (20) | SEQ-ID NO.: 72 | NP | HTL, CTL |
| KHWPQIAQF (19) | SEQ-ID NO.: 73 | NP | B cell |
| KIYSKHTPI (17) | SEQ-ID NO.: 74 | S1 | CTL |
| LSPRWYFYY (20) | SEQ-ID NO.: 75 | NP | HTL, CTL |
| NGVGYQPYR (6) | SEQ-ID NO.: 76 | S2 | HTL, CTL |
| NLNESLIDL (16) | SEQ-ID NO.: 77 | S2 | B cell, CTL |
| PYRVVVLSF (6) | SEQ-ID NO.: 78 | S2 | HTL, CTL |
| QYIKWPWYI (15) | SEQ-ID NO.: 79 | S2 | B cell, HTL, CTL |

TABLE 7
SARS-CoV-2 Polypeptide Combination medication

| | | Identification | |
|---|---|---|---|
| No. | Amino Acid Complex | number | Pep No. |
| 1 | LLFNKVTLADAGFIKQYGDCLGDIAA | SEQ-ID NO.: 38 | Pep-1 |
| 2 | KCYGVSPTKLNDLCFTNVY | SEQ-ID NO.: 39 | Pep-2 |
| 3 | LDSKVGGNYNYLYRLFRKSNLKPFER | SEQ-ID NO.: 40 | Pep-3 |
| 4 | FPRGQGVPI | SEQ-ID NO.: 41 | Pep-4 |
| | | | (reference) |
| 5 | SAAEASKKPRQKRTATKAYNVTQAFGRRPE | SEQ-ID NO.: 42 | Pep-5 |
| 6 | KFQPTNGVGYQPYRVVVLSFELLHAP | SEQ-ID NO.: 43 | Pep-6 |
| 7 | MAYRFNGIGVTQNVLYE | SEQ-ID NO.: 44 | Pep-7 |
| 8 | VSLVKPSFYVYSRVKNLNSSRVPDLLV | SEQ-ID NO.: 45 | Pep-8 |
| 9 | AATKMSECVLGQSKRVD | SEQ-ID NO.: 46 | Pep-9 |
| 10 | AISSVLNDILSRLDKVE | SEQ-ID NO.: 47 | Pep-10 |
| 11 | FFGMSRIGMEVTPSGTW | SEQ-ID NO.: 48 | Pep-11 |
| 12 | AFASEAARVVRSIFS | SEQ-ID NO.: 49 | Pep-12 |
| 13 | TFEYVSQPFLMDLEGKQ | SEQ-ID NO.: 50 | Pep-13 |
| 14 | GDCLGDIAARDLIC | SEQ-ID NO.: 51 | Pep-14 |
| 15 | LGKYEQYIKWPWYIWLGFIAG | SEQ-ID NO.: 52 | Pep-15 |
| 16 | EVAKNLNESLIDLQELGKYE | SEQ-ID NO.: 53 | Pep-16 |
| 17 | DGYFKIYSKHTPINLV | SEQ-ID NO.: 54 | Pep-17 |
| 18 | GKEDLKFPRGQGVPINTNSS | SEQ-ID NO.: 55 | Pep-18 |
| 19 | RQGTDYKHWPQIAQF | SEQ-ID NO.: 56 | Pep-19 |
| 20 | KDLSPRWYFYYLGTG | SEQ-ID NO.: 57 | Pep-20 |
| 21 | IRAAEIRASANLAATKM | SEQ-ID NO.: 58 | Pep-21 |

TABLE 8
SARS-CoV-2 Polypeptide Candidates M1-M19 determined with Bioinformatic Method

| No. | Amino Acid Complex | Identification number | No. (Pep No.) |
|---|---|---|---|
| M1 | LGKYEQYIKWPWYIWLGFIAGLIAIVM | SEQ-ID NO.: 80 | PepC-1 |
| M2 | LGKYEQYIKWPWYIWLG | SEQ-ID NO.: 81 | PepC-2 |
| M3 | LGKYEQYIKWPWYIWLGFIAG | SEQ-ID NO.: 52 | PepC-3 (Pep-15) |
| M4 | EVAKNLNESLIDLQELGKYE | SEQ-ID NO.: 53 | PepC-4 (Pep-16) |
| M5 | GDCLGDIAARDLIC | SEQ-ID NO.: 51 | PepC-5 (Pep-14) |
| M6 | KIYSKHTPINLVRD | SEQ-ID NO.: 82 | PepC-6 |
| M7 | LSRLDKVEAEVQID | SEQ-ID NO.: 83 | PepC-7 |
| M8 | LGQSKRVDFCGK | SEQ-ID NO.: 84 | PepC-8 |
| M9 | GKEDLKFPRGQGVPINTNSS | SEQ-ID NO.: 55 | PepC-9 (Pep-18) |
| M10 | IQGTDYKHWPQIAQF | SEQ-ID NO.: 56 | PepC-10 (Pep-19) |
| M11 | TFEYVSQPFLMDLEGKQ | SEQ-ID NO.: 50 | PepC-11 (Pep-13) |
| M12 | IRAAEIRASANLAATKM | SEQ-ID NO.: 58 | PepC-12 (Pep-21) |
| M13 | DGYFKIYSKHTPINLV | SEQ-ID NO.: 54 | PepC-13 (Pep-17) |
| M14 | DGYFKIYSKHTPINLVRD | SEQ-ID NO.: 85 | PepC-14 |
| M15 | KDLSPRWYFYYLGTG | SEQ-ID NO.: 57 | PepC-15 (Pep-20) |
| M16 | QYIKWPWYIWLGFI | SEQ-ID NO.: 86 | PepC-16 |
| M17 | VKQLSSNFGAISSVLN | SEQ-ID NO.: 87 | PepC-17 |
| M18 | KQLSSNFGAISSVL | SEQ-ID NO.: 88 | PepC-18 |
| M19 | AEIRASANLAATKMSECVLG | SEQ-ID NO.: 89 | PepC-19 |
| | QSKRVDFCGKGYH | | |
| M20 | AEIRASANLAATKMSECVLG | SEQ-ID NO.: 90 | PepC-20 |
| | QSKRVDF | | |

TABLE 9
Selection from most relevant and common MHC Class I and II Alleles worldwide

| MHC Class I alleles | MHC Class Il alleles |
|---|---|
| HLA-A*01:01, HLA-A*01:01, HLA-A*02:01, | HLA-DRB1*01:01, HLA-DRB1*03:01, HLA-DRB1*04:01, |
| HLA-A*02:01, HLA-A*02:03, HLA-A*02:03, | HLADRB1*04:05, HLA-DRB1*07:01, HLA-DRB1*08:02, |
| HLA-A*02:06, HLA-A*02:06, HLA-A*03:01, | HLADRB1*09:01, HLA-DRB1*11:01, HLA-DRB1*12:01, |
| HLA-A*03:01, HLA-A*11:01, HLA-A*11:01, | HLADRB1*13:02, HLA-DRB1*15:01, HLA-DRB3*01:01, |
| HLA-A*23:01, HLA-A*23:01, HLA-A*24:02, | HLADRB3*02:02, HLA-DRB4*01:01, HLA-DRB5*01:01, |
| HLA-A*24:02, HLA-A*26:01, HLA-A*26:01, | HLADQA1*05:01/DQB1*02:01, HLA-DQA1*05:01/DQB1*03:01, |
| HLA-A*30:01, HLA-A*30:01, HLA-A*30:02, | HLA-DQA10301-DQB10302, HLA-DQA1*04:01-DQB1*04:02, |
| HLA-A*30:02, HLA-A*31:01, HLA-A*31:01, | HLA-DQA1*01:01/DQB1*05:01, HLA-DQA1*01:02/DQB1*06:02, |
| HLA-A*32:01, HLA-A*32:01, HLA-A*33:01, | HLA-DPA1*02:01/DPB1*01:01, HLA-DPA1*01:03/DPB1*02:01, |
| HLA-A*33:01, HLA-A*68:01, HLA-A*68:01, | HLA-DPA1*01:03/DPB1*04:01, HLA-DPA1*03:01/DPB1*04:02, |
| HLA-A*68:02, HLA-A*68:02, HLA-B*07:02, | HLA-DPA1*02:01/DPB1*05:01, HLA-DPA1*02:01/DPB1*14:01 |
| HLA-B*07:02, HLA-B*08:01, HLA-B*08:01, | |
| HLA-B*15:01, HLA-B*15:01, HLA-B*35:01, | |
| HLA-B*35:01, HLA-B*40:01, HLA-B*40:01, | |
| HLA-B*44:02, HLA-B*44:02, HLA-B*44:03, | |
| HLA-B*44:03, HLA-B*51:01, HLA-B*51:01, | |
| HLA-B*53:01, HLA-B*53:01, HLA-B*57:01, | |
| HLA-B*57:01, HLA-B*58:01, HLA-B*58:01 | |

### Example 1: Antigen Response in Mice First Trial

4 Groups of wild-type mice (C57BL/6) consisting of 5 mice (group 2-4) and 3 mice for the negative control (group 1) were administered different vaccine candidates (as following) for evaluation.

The Groups were administered the following compositions:
a) Group 1 (no vac.): no vaccine
b) Group 2 (Pep X): peptide mix (60 μg per single peptide, 12 peptides selected from SEQ ID NOs: 38-49) with Pam3Cys (10 μg/dose) adjuvant
c) Group 3 (Pep C): peptide mix (60 μg per single peptide, 12 peptides selected from SEQ ID NOs: 38-49) with c-di-AMP (10 μg/dose) adjuvant
d) Group 4 (mRNA): Comirnaty mRNA vaccine (5 μg/dose)

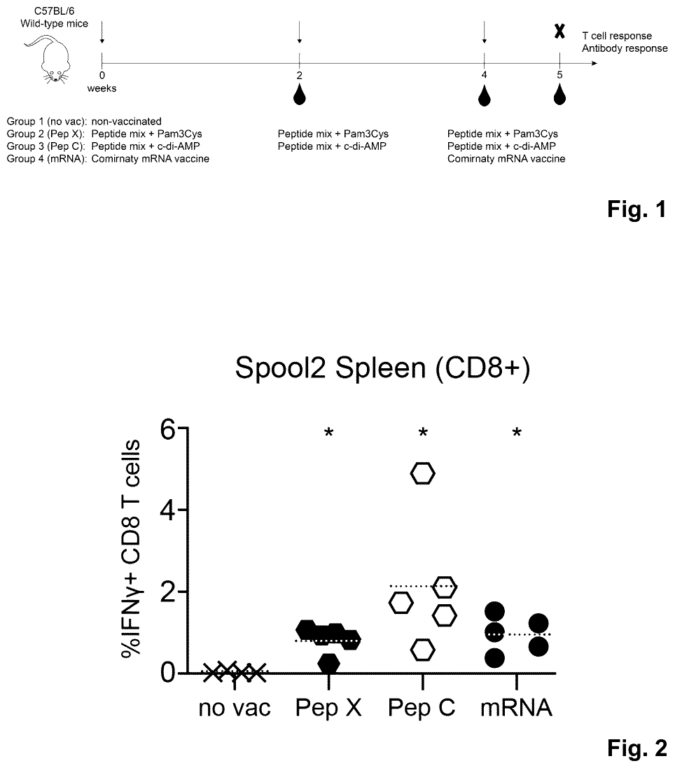

The antigen peptide formulations in accordance with the present invention (Groups 2 and 3) were administered subcutaneous (SC) three times in two-week intervals (see FIG. 1), the mRNA vaccine was administered intramuscular (IM) two times in a four-week interval (Group 4). After five weeks the immune response was determined, T cell CD4 and CD8 activity was examined by cytokines secreted in response to stimulation with the peptide antigens were determined by intracellular cytokine staining (ICS) with Intracellular cytokine staining with IFNγ, TNFα, and IL-2. Specific antibodies.

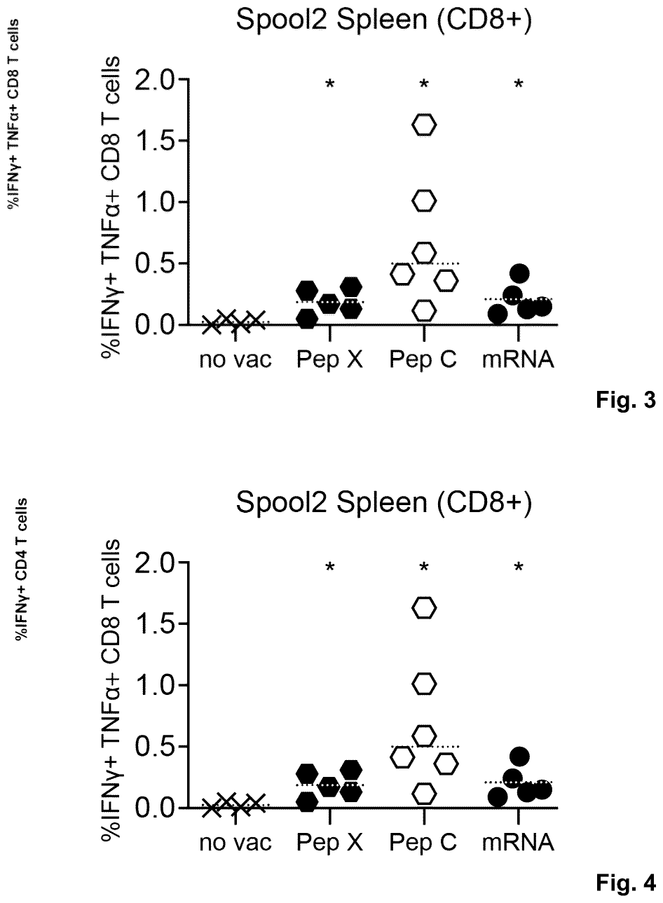

The immunization with a combination preparation in accordance with the present invention, particularly with Pep X and Pep C induced strong and multifunctional Spool2 and peptide mix specific CD8 T cell responses in comparison with the no vac. group 1 and even a greater response than the Comirnaty mRNA vaccine (FIG. 2 and FIG. 3). Immunization with Bis-(3′,5′)-cyclic-adenosine monophosphate (c-di-AMP) adjuvant (Pep C) induced a stronger CD8 T cell response than Pep X.

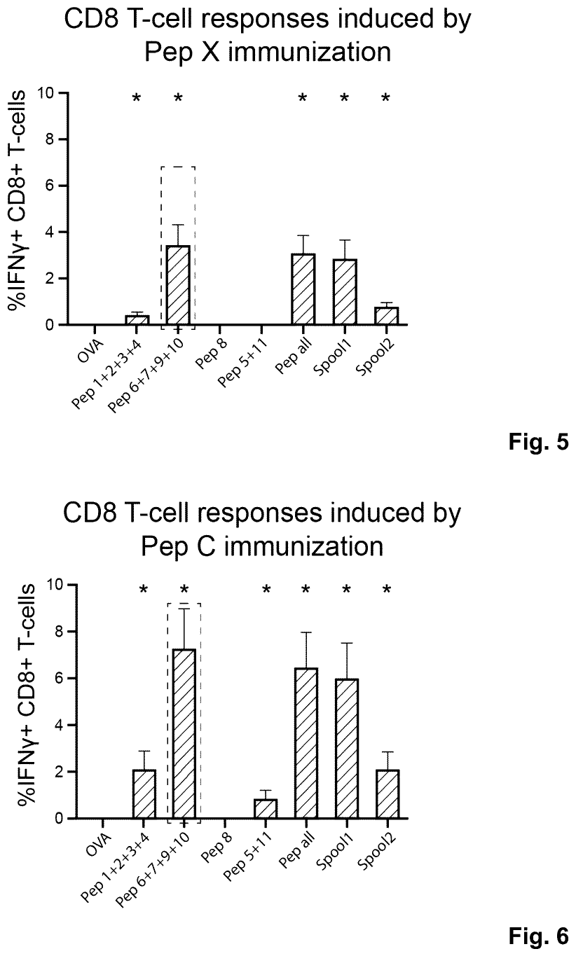

The immunization with a combination preparation in accordance with the present invention, particularly with Pep X and Pep C (Groups 2,3) also induced a stronger CD4 T cell response than the mRNA vaccination (Group 4) and the control group (FIG. 4). The CD8 T cell responses induced by the combination preparation, in particular comprising antigen peptide from SEQ ID NOs.: 38-49 were mostly spike protein S2 domain specific responses (FIG. 5,6). In the Pep X composition, the peptide combination of Pep 6+7+9+10 (SEQ ID NO.: 43, 44, 46, 47) induced a moderate CD8 T cell reaction (FIG. 5), while Pep 6+7+9+10 (SEQ ID NO: 43, 44, 46, 47) in the Pep C composition provoked a stronger response and Pep 1-4 (SEQ ID NO.: 38-41) and Pep-5+11 (SEQ ID NO.: 42, 48) showed some CD8+ activity (FIG. 6).

### Example 2: Antigen Response in Mice Second Trial

3 Groups of wild-type mice (C57BL/6) consisting of 5 mice (group 2,3) and 3 mice for the negative control (group 1) were administered different vaccine candidates (as following) for evaluation.

The Groups were administered the following compositions:
a) Group 1 (no vac.): no vaccine
b) Group 2 (Pep2 C): peptide mix (60 μg per single peptide, 12 peptides selected from SEQ ID NOs: 43, 44, 46, 47, 50-58) with c-di-AMP (10 mg/dose) adjuvant
c) Group 3 (mRNA): Comirnaty mRNA vaccine (5 μg/dose)

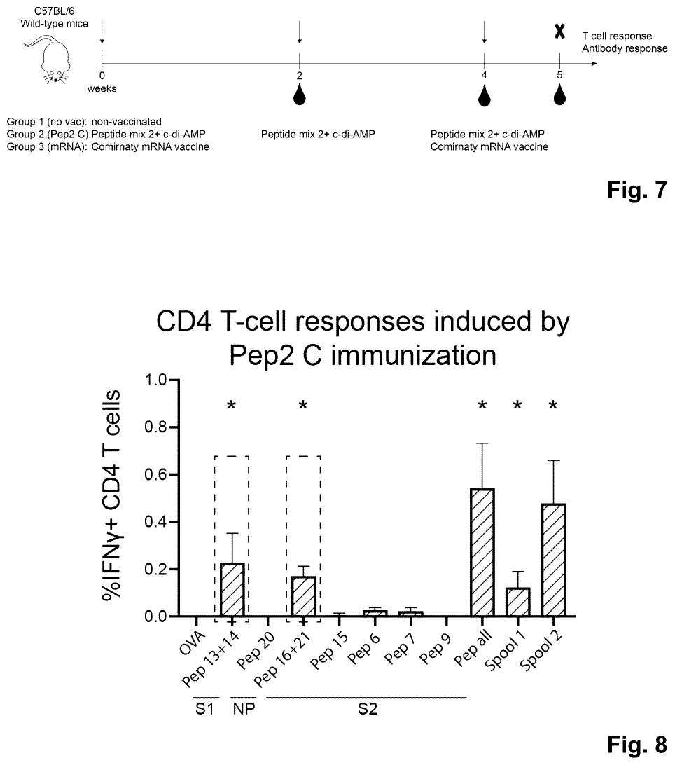

The antigen peptide formulations in accordance with the present invention were administered subcutaneous (SC) three times in two-week intervals (see FIG. 7), the mRNA vaccine was administered intramuscular (IM) two times in a four-week interval (Group 3). After five weeks the immune response was determined, T cell CD8 activity was examined by cytokines secreted in response to stimulation with the peptide antigens were determined by intracellular cytokine staining (ICS) with Intracellular cytokine staining with IFNγ and TNFγ. Specific antibodies.

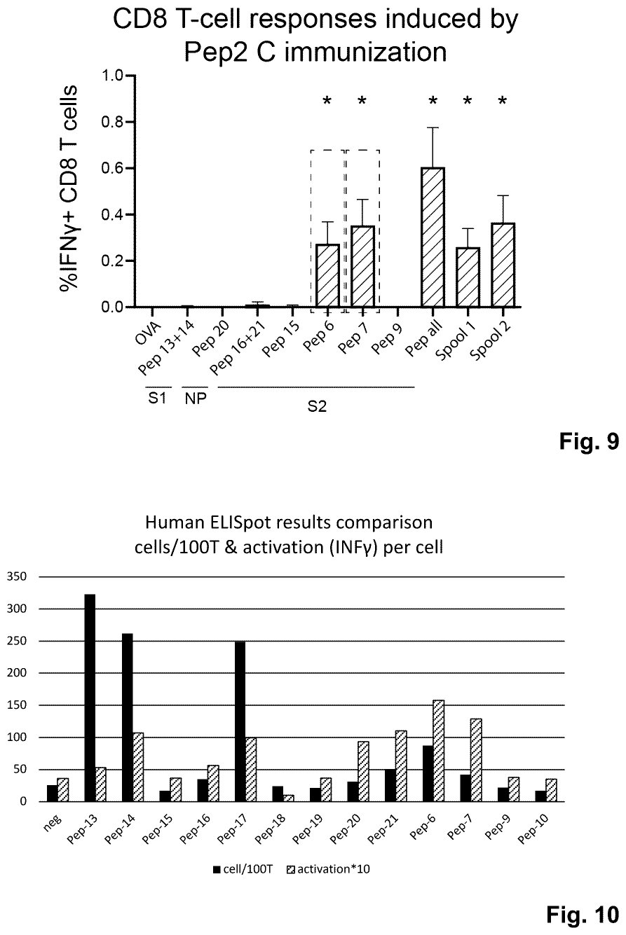

The CD4 T cell responses induced by the combination preparation, in particular comprising antigen peptide from SEQ ID NOs.: 43, 44, 46, 47, 50-58 were mostly spike protein S1 and S2 domain specific responses. The different peptides of the pharmaceutical composition show different CD4 and CD8 activation selectivity. While the peptides Pep 13+14 (SEQ ID NOs.: 50-51) and Pep 16+21 (SEQ ID NOs.: 50-51) caused a moderate CD4 T cell reaction. (FIG. 8), the peptides Pep 6 (SEQ ID NO.: 43) and Pep 7 (SEQ ID NO.: 44) caused a CD8 T cell reaction (FIG. 9). The CD8 T cell responses induced by the combination preparation, in particular comprising antigen peptide from SEQ ID NOs: 43, 44, 46, 47, 50-58 were mostly a spike protein S2 domain specific response.

### Example 3: IFNγ Activity of Antigen Peptides in Human Blood Serum

IFNγ ELISpot analysis was performed ex vivo (without further in vitro culturing for expansion) using PBMCs from a healthy adult human. The cells of the healthy donor were pre-stimulated with the pool of Covid peptides. Then the cells were stimulated in the ELISpot with the individual peptides, all peptide wells were prepared as duplicates, except for blind test and positive control which were performed as single wells. 267,000 cells per well were used.

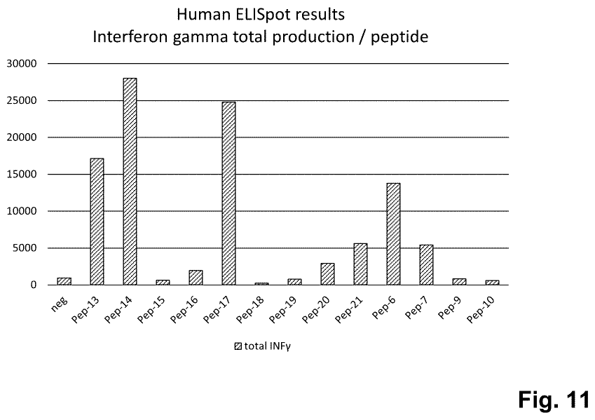

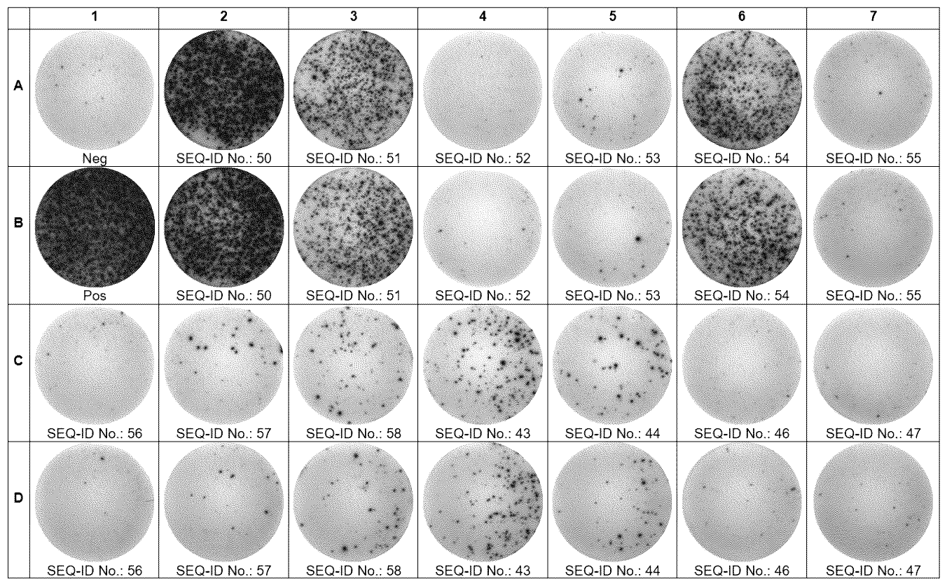

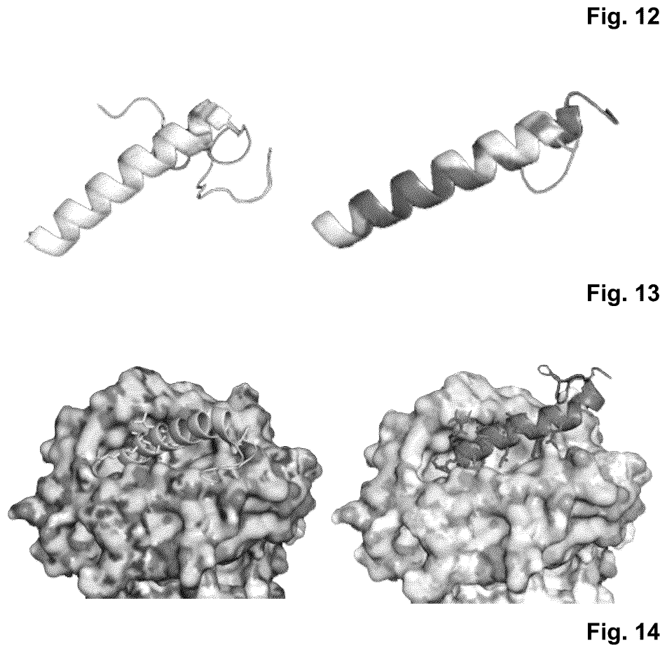

The positive counts per 100,000 cells and activation values times 10 for the antigen peptides as analyzed with ELISpot (FIG. 11) are noted below.

| Peptide | SEQ ID NO. | Cells/100,000 cells | Activation*10 |
|---|---|---|---|
| neg | — | 25.8 | 36.0 |
| Pep-13 | SEQ-ID NO.: 50 | 322.8 | 53.1 |
| Pep-14 | SEQ-ID NO.: 51 | 262.2 | 106.9 |
| Pep-15 | SEQ-ID NO.: 52 | 16.9 | 36.5 |
| Pep-16 | SEQ-ID NO.: 53 | 34.8 | 56.2 |
| Pep-17 | SEQ-ID NO.: 54 | 249.1 | 99.6 |
| Pep-18 | SEQ-ID NO.: 55 | 24.3 | 10.1 |
| Pep-19 | SEQ-ID NO.: 56 | 21.3 | 36.6 |
| Pep-20 | SEQ-ID NO.: 57 | 31.5 | 93.3 |
| Pep-21 | SEQ-ID NO.: 58 | 50.9 | 110.4 |
| Pep-6 | SEQ-ID NO.: 43 | 87.3 | 157.8 |
| Pep-7 | SEQ-ID NO.: 44 | 41.9 | 129.0 |
| Pep-9 | SEQ-ID NO.: 46 | 22.1 | 38.0 |
| Pep-10 | SEQ-ID NO.: 47 | 17.2 | 34.8 |

Peptides 13, 14 and 17 showed especially high counts per 100,000 cells, while the IFNγ activity was the highest for the antigen peptides Pep-6, Pep-7, Pep-21 and Pep-14 in descending order (FIG. 10), corresponding to the SEQ ID NOs.: 43, 44, 58, 51. The activation value is a measure of the cytokine activity and was calculated according to the following formula:

$${\sum }_{i = 1}^{n} \frac{( Size i * Intensity i )}{1000} .$$

The results are shown in FIG. 11, the effectiveness of immunization is (according to formula) the product of number of activated cells and activation formed, thus the most effective antigen peptides are (in descending order) Pep-14, Pep-17, Pep-13, Pep-6, Pep-21, and Pep-7, corresponding to the SEQ ID NOs.: 51, 54, 50, 43, 58, 44. All of these peptides have an activation over 75, which is the threshold (3 times negative control) for a positive immunization effect.

### Example 4: Vaccination of a Group of Healthy Patients with Risk of Contracting a Coronavirus

All subjects (hereinafter also referred to as patients) are adults, preferably human adults. The vaccine consisting of the pharmaceutical composition according to the invention is administered as described, preferably intramuscularly. The pharmaceutical composition comprises a pharmacologically effective amount (600 μg per peptide per vial) of the 12 antigen peptides (SEQ ID NO: 38-58) and the adjuvant c-di-AMP (bis-(3′,5′)-cyclic-adenosine dissolved or suspended in water: DMSO 75%: 25% (vol %: vol %). The administration is repeated two times in two-week intervals (FIG. 1), for a total of three administrations in four weeks. This ensures an optimal CD4 and CD8 T cell response.

### Example 5: Bioinformatic Determination of Pharmaceutically Active HLA Antigen Peptides and/or Antigen Polypeptides

Step (a): Bioinformatics expert system: In order to bioinformatically determining the amino acid sequences of the HLA antigen peptides of the SARS-COV-2 corresponding to the MHC complexes (nonamers) with the highest allelic affinities (specific activity towards T cell receptor [nM]) were first selected from the dataset of the SARS-COV-2 virus proteins (nucleoprotein, UNIPROT Reference ID sequence: PODTC9, and spike protein, UNIPROT Reference ID sequence: PODTC2) based on the amino acid sequences of the respective proteins. The highest conserved regions in relation with other human coronaviruses and non-variant mutations were selected from proteins with a combination of different bioinformatic methods to further analyze. To this scope, the Nucleocapsid protein was aligned against 5 coronaviruses with COBALT tool (Human coronavirus 229E, UNIPROT reference: P15130; Human coronavirus OC43, UNIPROT reference: P33469, Human coronavirus HKU1 (isolate N5), UNIPROT reference: QOZME3; Human coronavirus HKU1 (isolate N1), UNIPROT reference: Q5MQC6; and severe acute respiratory syndrome coronavirus 2, UNIPROT reference: Q6S8E1) according to a conservation algorithm based on the relative entropy threshold of the residue. SARS-COV-2 nucleocapsid protein positions 59-84, 86-96, 99-123, 129-142, 155-170, 284-320, 321-336, 342-360 are highly conserved. These regions are used for further binding affinity prediction. SARS-COV-2 spike protein was aligned against 8 coronaviruses (Human coronavirus 229E, UNIPROT reference: P15423; Human coronavirus OC43, UNIPROT reference: P36334, Human coronavirus HKU1 (isolate N5), UNIPROT reference: QOZME7; Human coronavirus HKU1 (isolate N1), UNIPROT reference: Q5MQD0; Severe acute respiratory syndrome coronavirus, UNIPROT reference: P59594; Human Coronavirus NL63, UNIPROT reference: Q6Q1S2; MERS-COV, UNIPROT reference: K9N5Q8; and Human coronavirus HKU1 (isolate N2), UNIPROT reference: Q14EB0). SARS-CoV-2 spike protein positions 152-211, 470-503, 534-570, 769-847, 939-1063 and 1176-1273 are highly conserved. Then, the selection criterion is used to algorithmically determine the probability with which the respective HLA antigen peptide is presented in vivo on the corresponding MHC complexes of the SARS-COV-2 virus (a first prerequisite for a possible T cell immune response). ADE and B/T cell response balancing SARS-COV-2 peptides was made using a variety of in silico methods, 3D modeling, and binding prediction test the effect of different antibodies on the conformational change of the SARS-COV-2 spike protein and select peptides that are less likely to trigger ADE. Artificial intelligence such as the neural network NNAlign used in the publicly available IEDB server is used for information processing, and the mathematical modeling and biotechnological advancements based on it are used for experimental analysis on the causality of a pharmaceutical composition of the vaccine. From the dataset of the SARS-COV-2 virus proteins (nucleoprotein, UNIPROT Reference ID sequence: PODTC9, and spike protein, UNIPROT Reference ID sequence: PODTC2) nonameric variants involving amino acid exchange were determined with respect to 12 of the highest affinities (specific activity toward T cell receptor [nM]) based on the patient's alleles. Also, polymers of 17 amino acids (oligopeptides) were determined under the affinity criterion for this purpose. This selection criterion is used to algorithmically determine the probability of presenting the respective antigen peptide corresponding to the MHC complexes in vivo on the corresponding MHC complexes (a 15 second requirement for a possible cellular immune response).

Step (b): Bioinformatics expert system: Based on the new data sets identified in step (a) with the addition of the HLA-restricted ligands (the data set of the ligandome), a selection of HLA antigen peptides corresponding to the MHC class I complexes and corresponding to the MHC class II complexes was made, which were the most promising epitope candidates for eliciting a cellular immune response, both individually and, most importantly, 12 in combination. This allows existing knowledge about ADE, including that within the existing COVID-19 Disease Map Collaboration, to be extracted from the extensive literature about FIPV2,3, MERS4,5, and SARS and tested for gaps through modeling: Knowledge gaps can be narrowed by simulation-based hypothesis testing to the point where experimental testing of the remaining variants becomes feasible.

Step (c): AI and machine learning: Natural language processing (NLP) such as Natural Language Toolkit (NLTK) is used to extract further information to reinforce the predictions from steps (a) and (b), by learning patterns that allow information linkage to be identified or that allow tables and figures to be included in the evaluation. To further improve on the mathematical model, relevant information on molecular and cellular interaction networks between coronaviruses and the host can be extracted from the total amount of scientific literature and related to ADE. Biological processes like signal transduction, gene regulation and metabolism are accounted and integrated into the ADE prevention by artificial intelligence by translation into a mathematical model that maps the molecular mechanisms.

Step (d): Biotechnology platforms: The effectiveness of the peptide immunizations is tested with high throughput biotechnological methods such as rapid peptide synthesis (Merrifield solid-phase peptide synthesis, Bailey peptide synthesis, N-carboxylic anhydride method) and rapid immunogenicity assays (Western Blot, Electrochemiluminescent immunoassay (ECL), ELISA techniques, intracellular cytokine staining (ICS), ELISPOT, AFM, MS, see FIG. 2-6). These techniques ensure that conformation-specific SARS-COV-2 antibody and a balanced B/T-cell reaction as predicted with in silico methods (a)-(c) are archived by the pharmaceutical composition comprising of at least one HLA antigen peptide in accordance with the present invention is found.

### Example 6: Bioinformatic Determination of Pharmaceutically Active HLA Antigen Peptides and/or Antigen Polypeptides for Prophylaxis or Treatment of a Virus Infectious Disease Such as SARS-COV-2

An example of a tandem polypeptide pool generation for the use in the prophylaxis and/or treatment of a virus infectious disease, the optimization was based on the SARS-COV-2 viral disease. A method according to one embodiment of the current invention was used, containing four steps a-e):

b) The conservative regions of the virus were determined with two sequence comparison tools, MUSCLE (Multiple Sequence Comparison by Log-Expectation) and COBALT. It is aligned according to a conservation algorithm based on the relative entropy threshold of the residue. The regions will be distinguished into highly conserved and less highly conserved based on the number of exchanges and substitutions in the amino acid sequence. In the next step first only highly conserved regions will be used. The amino acid sequence of the nucleocapsid protein (in FASTA Format) (ID: PODTC9) of the Severe Acute Respiratory Syndrome Coronavirus 2 (SARS-COV-2, 2019-nCOV; amino acids 1-419) were compared with 5 other coronavirus strains: Human Coronavirus OC43 (ID: P33469), Human SARS Coronavirus (SARS-COV) (ID: P59594), Human Coronavirus 229E (HCoV-229E) (ID: P15130), human coronavirus HKU1 (HCoV-HKU1, isolate N5) (ID: QOZME3), human coronavirus HKU1 (HCoV-HKU1, isolate N1) (ID: Q5MQC6), and severe acute respiratory syndrome coronavirus (ID: Q6S8E1). The following regions were determined as conserved between these virus strains: 152-211, 470-503, 534-542 (570), 769-847, 939-1063, and 1176-1273. The amino acid sequence of the spike S protein (ID: PODTC2) (in FASTA Format) of the Severe Acute Respiratory Syndrome Coronavirus 2 (SARS-COV-2, 2019-nCOV; amino acids 1-1723) were compared with 5 other coronavirus strains: Human coronavirus NL63 (HCoV-OC43) (ID: Q6Q1S2), human coronavirus OC43 (ID: P33469), human SARS coronavirus (SARS-COV) (ID: P59594), human coronavirus 229E (HCoV-229E) (ID: P15423), middle east respiratory syndrome-related coronavirus (ID: K9N5Q8), human coronavirus HKU1 (HCoV-HKU1, isolate N5) (ID: QOZME3), human coronavirus HKU1 (HCoV-HKU1, isolate N1) (ID: Q5MQC6), human coronavirus HKU1 (HCoV-HKU1, isolate N2) (ID: Q14EB0) and severe acute respiratory syndrome coronavirus (ID: Q6S8E1). The following regions were determined as conserved between these virus strains: 152-211, 470-503, 534-542 (570), 769-847, 939-1063 and 1176-1273. The recent SARS-COV-2 viral mutants alpha (B.1.1.7), beta (B.1.351), gamma (P. 1), delta (B.1.617.2) and omicron (B.1.1.529) were compared with these regions, therefore, the region 470-503 from the spike protein was removed from the list of conserved regions.

c) In the next step B-cell and T-cell epitope alignment was determined from the amino acid sequences from the conserved regions. The linear B-cell binding prediction was done with BepiPred (or BCell IEDB), SVMtrip, ABCPred and BCPreds, the threshold for a B-cell epitope matching candidate was equal or above 0.5 (percent value of accuracy of the ML model in matching a peptide to the epitope). Any portion of the conserved region with a score of 0.8 and higher is considered a B-cell epitope very good match. The amino acid sequences are then expanded algorithmically to match multiple B-cell epitopes, where possible, creating tandem polypeptides. The cytotoxic T lymphocytes (CTL, CD8) binding prediction is done with NetMHCpan 4.1 server from IEDB. From the whole protein sequence 9-10 amino acid length epitopes that match MHC class I complexes are obtained. The threshold (NetMHC threshold) for a positive hit is a peptide rank of 2 or less. Then the result amino acid chains are expanded to include more MHC class I matching sequences in the same amino acid chain, where possible. The helper T lymphocytes (HTL, CD4) binding prediction is done with NetMHCIIpan and consensus server from IEDB server to predict the potential CTL epitopes by comparing the conservative region with the most common HLA class II alleles (worldwide population, see table 9). From the whole protein sequence epitopes 12-18 amino acids in length that match MHC class II complexes are obtained. Preferably, the threshold for a weak binding allele (NetMHC threshold) is equal to 10 or less and for a strong binding, the binding affinity is equal to two or less. The virus epitope is than prolongated to cover more HTL alleles, the CTL scores are secondary in this case. This is done until the amino acid sequence pool targets at least 95% of the worldwide population. The prediction of the HTL epitope match and CTL epitope match is again performed in parallel with prediction of B-cell epitope match using NetMHC (CTL), NetMHCpan (HTL and CTL), and consensus (HTL) and a final comparison step using MHCPred. Preferably, highly conserved regions are used for prediction, but can be extended to moderately conserved regions if the inclusion of a longer amino acid sequence improves binding results or allows the inclusion of more HTL alleles. Candidates are expanded according to optimize the parallel result score with an optimization algorithm and/or neural network that maximizes binding affinities at all scores (HTL, CTL, and B-cell binding) by including longer sequences of the viral epitope. The algorithm and/or neural network also optimizes the protein coverage of the viral epitope to bring together sequences from different parts of the virus into one polypeptide. Therefore, it is possible to improve the prediction threshold for HTL match to 5 or less and for CTL match to 1 or less. The results of this optimization run are listed in Table E6-1 and E6-2.

d) Following the amino acid sequence generation in step c), the physiological and physicochemical properties are determined and evaluated. The preselected amino acid sequences from step a-c) are tested for antigenicity with the compatibility algorithms VaxiJen and/or ANTIGENpro, and are then tested with the allergenicity prediction algorithms AllerTOP, and/or AllergenFP, an amino acid sequence is considered not allergenic if the Tanimoto index is below 0.7. The physicochemical properties of the amino acid sequences from a-c) are determined. The solubility of the peptides is determined using SolPro, which can be run with any length of amino acid, the toxicity potential is determined using ToxinPred, and other physicochemical properties such as hydrophobicity, pl, charge, molecular weight, which are important for the synthesis of these peptides, are determined using any means known to those skilled in the art. The results are listed in Table E6-3.

e) Tandem polypeptide selection and in vivo feedback. The amino acid sequences, also referred to as antigen polypeptides, as determined in step a-d) are weighted by a in house machine learning (ML) model, which weights first HLA alleles coverage, T-cell and B-cell binding affinity scores and optimized across the three, while also considering the protein coverage and optimizing the distribution of the combination peptides across different proteins of a viral epitope, such as the nucleocapsid, spike S1 and S2 proteins. The in-house machine learning (ML) approach predicts the immunogenicity, which is verified by the results from in vivo feedback. In a preferred embodiment, the ML is thus continuously improved by feedback from in vivo immunogenicity studies such as elispot and ELISA. The immunogenic epitopes are predicted based on an in-house ML model, since the binding affinity determined in accordance with the current invention as specified in the previous step b) is not accurate enough in itself to predict whether a polypeptide of the invention is immunogenic or not.

The prediction of the immunogenicity of an antigen polypeptide matching a viral epitope was done using an SVM-based model which was trained with the IEDB database (human binding epitopes tested by Elispot and ICS). The accuracy of the prediction is more than 65% and is further improved by epitope binding affinity calculations as used in b) to remove false positives. The model considers the physicochemical properties of the polypeptides such as hydrophobicity, pl, charge, molecular weight molecular weight, which are important for the synthesis of these peptides. For the antigen polypeptides, it is important to consider the molecular weight, pl and charge at pH 5-6 and GRAVY, since according to the invention, intermediate and polar polypeptides are used at subcutaneous pH (5-6) to avoid precipitation and as well as preferential weighting for hydrophobic peptides (positive GRAVY results).

The immunogenicity is calculated using IEDB, the TAP proteosome determined with NetCTLpan, and the cleavage determined with NetChop. The trained ML model correlation data between inputs and outputs can be quantified and is obtained in proportions of 0 to |1|. Therefore, it is possible to quantify the parameters which are particularly influential in the polypeptide selection, thus allowing for further, step by step improvement of the data.

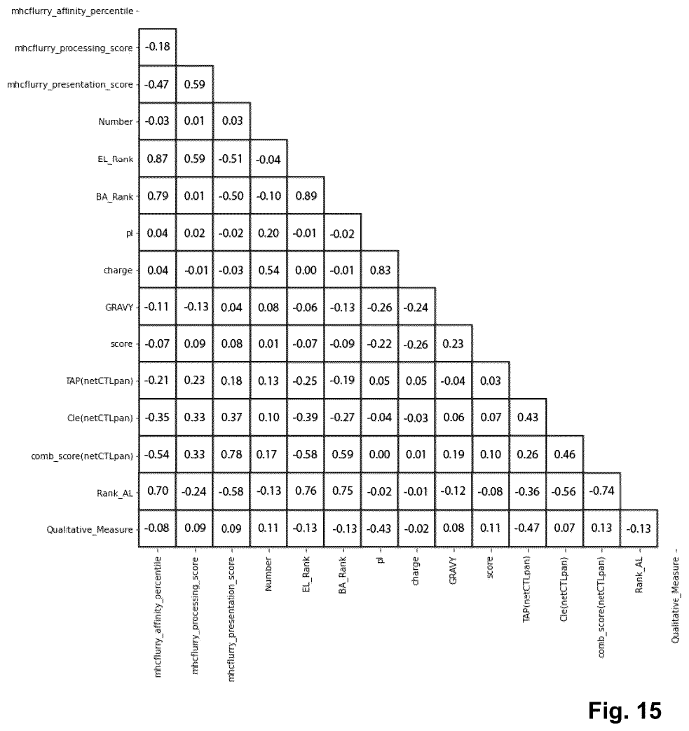

FIG. 15 shows the ML model trained with IEDB training data and the correlation plot of the input-output between different entries. The correlation is given in steps between-1 and 1. Correlations include the results of in silico binding affinity, TAP proteosome, cleavage, physicochemical properties, and immunogenicity (Qualitative_measure), as determined by Elispot and ICS tested in vitro. MHCflurry inputs are mhcflurry_affinity_percentile, mhcflurry_processing_score and mhcflurry_presentation_score. Number represents the number of cleavage points found. EL_Rank and BA_Rank are results from the NetMHCpan of mass spectrometry-eluted ligand (EL) and quantitative binding affinity prediction (BA) peptides. The model includes physicochemical properties such as pl, charge, and GRAVY. The score input is the result of the immunogenicity tool of IEDB validated only for 9-mer peptides. The TAP proteosome information is in the TAP entry, the probability that the peptide is cleaved in the N-terminal cle, comb_score is the result of cleavage, TAP and binding affinities from netMHCpan and the rank of all is Rank_AL.

The following thresholds have been introduced on the preselection of amino acids for the weighting, explicitly on allergenicity, solubility, human proteome alignment and toxicity. Amino acid sequences where excluded which had antigenic predictions with a value below 0.4, toxic epitopes, peptides with higher similarity than 35% to human proteome and non-soluble peptides with a solubility value below 0.5. Only amino acid sequences with HTL matching below 5 and sequences matching alleles with CTL matching below 1 were included. The highest weighted factor is the binding affinity in the B-cell prediction (factor 1), then the coverage results for HTL 5 (factor 0.7) and number of alleles in CTL (factor 0.7).

Using the bioinformatic method in accordance with the current invention, thus the sample pool of peptides and combination peptides corresponding to HLA antigen peptides were preselected in step a-d) (Table 8, PepC-1-PepC-18).

Through the optimization process in step e) based on information of d), the peptides M3-5, M9-13 and M15 were selected for in-vivo tests as described in Example 3. The selection has an HTL coverage of 98.65 of the worldwide population represented by the common MHC Class I and Class II alleles listed in Table 9.

TABLE E6-1
Chemical/Biochemical properties of the peptides M1-M18.

| No. | Protein | length | Bcell | Rep | HTL | CTL | Charge | pI | GRAVY | Mol Weight |
|---|---|---|---|---|---|---|---|---|---|---|
| M1 | S2 | 27 | >0.8 | 4 | 8 | 14 | 1 | 8.38 | 0.822 | 3272.98 |
| M2 | S2 | 17 | >0.8 | 4 | 2 | 12 | 1 | 8.38 | −0.424 | 2243.64 |
| M3 | S2 | 21 | >0.8 | 4 | 3 | 12 | 1 | 8.38 | 0.071 | 2632.1 |
| M4 | S2 | 20 | 0.565 | 1 | 1 | 11 | −3 | 4.25 | −0.63 | 2305.57 |
| M5 | S2 | 14 | 0.7 | 0.5 | 0 | 1 | −2 | 3.93 | 0.671 | 1434.65 |
| M6 | S1 | 14 | 0.64 | 0.5 | 1 | 11 | 2 | 9.7 | −0.707 | 1683.97 |
| M7 | S2 | 14 | 0.993 | 0.17 | 1 | 4 | −2 | 4.32 | −0.314 | 1614.82 |
| M8 | S2 | 12 | 0.58 | 0.33 | 0 | 0 | 2 | 9.31 | −0.633 | 1337.56 |
| M9 | NP | 20 | 0.977 | 0.5 | 0 | 0 | 1 | 8.6 | −1.06 | 2144.34 |
| M10 | NP | 14 | 0.86 | 0.63 | 0 | 0 | 1 | 6.74 | −1.21 | 1718.86 |
| M11 | S1 | 17 | 0 | — | 4 | 6 | −1 | 4.14 | −0.41 | 2032.27 |
| M12 | S2 | 18 | 0 | — | 8 | 9 | 2 | 10.8 | 0.24 | 1787.09 |
| M13 | S1 | 16 | 0 | — | 3 | 14 | 2 | 8.44 | −0.27 | 1895.16 |
| M14 | S1 | 18 | 0.64 | 0.5 | 3 | 14 | 1.5 | 8.44 | −0.682 | 2166.46 |
| M15 | NF | 15 | 0 | — | 2 | 13 | 1 | 8.43 | −0.68 | 1866.08 |
| M16 | S2 | 14 | 0 | — | 2 | 7 | 1 | 8.5 | 0.39 | 1913.26 |
| M17 | S2 | 16 | 0.67 | | 1 | 2 | 1 | 8.72 | 0.44 | 1663.87 |
| M18 | S2 | 14 | 0.67 | | 1 | 2 | 1 | 8.75 | 0.46 | 1450.64 |

TABLE E6-2
HTL coverage and corresponding alleles of the peptides M1-M18.

| No. | HTL coverage | HTL alleles |
|---|---|---|
| M1 | 93.97 | HLA-DQA1*01:01/DQB1*05:01, HLA-DQA1*05:01/DQB1*03:01, HLA- |
| | | DPA1*01:03/DPB1*02:01, HLA-DPA1*02:01/DPB1*01:01, HLA- |
| | | DPA1*01:03/DPB1*04:01, HLA-DRB1*12:01, HLA-DRB1*01:01, HLA- |
| | | DRB1*09:01 |
| M2 | 69.75 | HLA-DPA1*01:03/DPB1*04:01, HLA-DPA1*01:03/DPB1*02:01 |
| M3 | 84.81 | HLA-DPA1*01:03/DPB1*04:01, HLA-DPA1*01:03/DPB1*02:01, HLA- |
| | | DPA1*02:01/DPB1*01:01 |
| M4 | 26.43 | HLA-DQA1*03:01/DQB1*03:02 |
| M5 | 0 | — |
| M6 | 18.23 | HLA-DRB1*07:01 |
| M7 | 26.43 | HLA-DQA1*03:01/DQB1*03:02 |
| M8 | 0 | — |
| M9 | 0 | — |
| M10 | 0 | — |
| M11 | 88.62 | HLA-DPA1*01:03/DPB1*04:01, HLA-DPA1*01:03/DPB1*02:01, HLA- |
| | | DPA1*02:01/DPB1*01:01, HLA-DPA1*03:01/DPB1*04:02 |
| M12 | 75.68 | HLA-DPA1*02:01/DPB1*14:01, HLA-DQA1*01:02/DQB1*06:02, HLA- |
| | | DRB3*02:02, HLA-DRB1*13:02, HLA-DQA1*05:01/DQB1*03:01, HLA- |
| | | DRB1*04:01, HLA-DRB1*09:01, HLA-DRB1*08:02 |
| M13 | 42.33 | HLA-DRB1*08:02, HLA-DRB1*07:01, HLA-DRB1*08:02, HLA- |
| | | DPA1*02:01/DPB1*14:01 |
| M14 | 42.33 | HLA-DRB1*08:02, HLA-DRB1*07:01, HLA-DRB1*08:02, HLA- |
| | | DPA1*02:01/DPB1*14:01 |
| M15 | 75.36 | HLA-DQA1*01:01/DQB1*05:01, HLA-DPA1*01:03/DPB1*02:01 |
| M16 | 69.75 | HLA-DPA1*01:03/DPB1*04:01, HLA-DPA1*01:03/DPB1*02:01 |
| M17 | 0 | HLA-DRB3*02:02 |
| M18 | 0 | HLA-DRB3*02:02 |

TABLE E6-3
Biochemical/immunological properties of the peptides M1-M18.

| | | | Aller- | | | | | Antigen |
|---|---|---|---|---|---|---|---|---|
| No. | TH1 | TH2 | genicity | Blast | start | end | Vaxijen | Pro |
| M1 | − | ++ | 0/0 | 0.0 | 1192 | 1235 | 0.77 | — |
| M2 | + | + | 0/0 | 0.0 | 1192 | 1209 | 0.90 | — |
| M3 | + | + | 0/0 | 0.0 | 1192 | 1213 | 0.84 | — |
| M4 | + | − | 1/0 | 0.0 | 1176 | 1196 | 0.49 | 0.37 |
| M5 | − | − | 1/0 | 0.0 | 827 | 841 | 0.45 | 0.15 |
| M6 | − | ++ | 1/0 | 0.0 | 191 | 205 | 0.56 | 0.02 |
| M7 | − | + | 1/0 | 0.0 | 970 | 984 | 0.42 | 0.63 |
| M8 | + | + | 1/0 | 0.0 | 1023 | 1035 | 1.51 | 0.05 |
| M9 | + | + | 0/0 | 0.0 | 59 | 78 | 0.48 | 0.82 |
| M10 | + | − | 0/0.66 | 0.0 | 293 | 307 | 0.63 | 0.32 |
| M11 | + | ++ | 0/0.65 | 31.3 | 156 | 172 | 0.73 | 0.04 |
| M12 | + | ++ | 1/0.62 | 0.0 | 1002 | 1020 | 0.60 | 0.21 |
| M13 | + | ++ | 1/0 | 33.3 | 187 | 203 | 0.76 | 0.17 |
| M14 | + | ++ | 1/0 | 0.0 | 187 | 205 | 0.49 | — |
| M15 | − | ++ | 0/0 | 30 | 103 | 118 | 1.21 | 0.30 |
| M16 | ++ | No | 1/0 | 28.6 | — | — | 1.16 | — |
| M17 | ++ | No | 1/0 | 31.3 | — | — | 0.44 | — |
| M18 | ++ | No | 1/0 | 0 | — | — | 0.51 | — |

### Example 7: Intracellular Cytokine Staining (ICS) of Antigen Peptides and/or Polypeptides According to the Current Invention

An Intracellular Cytokine Staining (ICS) assay to assess the T-cell responses induced by an antigen polypeptide in the spirit of the current invention was carried out following this experimental procedure. The experimental procedure involved several sequential steps to prepare and analyze freshly harvested peripheral blood mononuclear cells (PBMCs) from a human donor.

PBMCs were cultured in RPMI-1640 medium supplemented with 10% fetal bovine serum, penicillin-streptomycin, and L-glutamine at 37° C. with 5% CO₂. After culture, the cells were divided into different experimental groups. The cells were then stimulated with the antigen peptide vaccine or a control peptide, along with appropriate cytokines and co-stimulatory molecules to facilitate T cell activation. The antigen peptide was designed to effectively stimulate both CD4 and CD8 T-cell responses.

To inhibit cytokine secretion and promote intracellular cytokine accumulation, brefeldin A was added to the stimulated cells at a concentration of 10 μg/mL, and the cells were incubated overnight. Subsequently, the cells were harvested, and staining was performed. Fluorescently labeled antibodies, including AlexaFluor 488-conjugated anti-IFNγ and APC-H7-conjugated anti-CD4, as well as Pacific Orange-conjugated anti-CD8 and a cell surface marker were added to the cells and incubated.

To facilitate antibody access to intracellular molecules, the cells were fixed and permeabilized using a fixation solution and permeabilization buffer. Intracellular cytokine staining was carried out by adding fluorescently labeled antibodies against IFNγ or other cytokines of interest. Following staining, flow cytometry analysis was performed by gating on CD4 and CD8 T cells to determine the percentage of cytokine-producing cells.

The obtained data were analyzed to compare the percentage of cytokine-producing CD4 and CD8 T cells stimulated with the antigen polypeptides to that of unstimulated CD4 and CD8 T cells or T cells stimulated with a control peptide. Statistical tests, such as t-tests or ANOVA, were used to assess significant differences between groups.

The following table (Table E7-1) lists antigen polypeptides, as single antigen polypeptides or as a pharmaceutical composition in accordance with the current invention, tested according to the above:

TABLE E7-1
Antigen polypeptides tested with ICS

| Group Nr. | Identifier | Peptides | Comment |
|---|---|---|---|
| 1 | OVA | OVA (blank) | unrelated Peptide, for Ref. |
| 2 | Pep all | Pep-1-Pep-12 | 12 antigen polypeptides, see |
| | | (SEQ-ID NOs.: 38-49) | FIG. 5 and FIG. 6 |
| 3 | Pep 1 + 2 + 3 + 4 | Pep-1-Pep-4 | containing 1 and 4 for reference, |
| | | (SEQ-ID NOs.: 38-41) | see FIG. 5 and FIG. 6 |
| 4 | Pep 6 + 7 + 9 + 10 | Pep-6, Pep-7-Pep-10 | 4 antigen polypeptides, see FIG. |
| | | (SEQ ID NOs.: 43, 44-47) | 5 and FIG. 6 |
| 5 | Pep 5 + 11 | Pep-5, Pep-11 | 4 antigen polypeptides, see FIG. |
| | | (SEQ-ID NOs.: 42, 48) | 5 and FIG. 6 |
| 6 | Pep 13 + 14 | Pep-13, Pep-14 | see FIG. 8 and FIG. 9 |
| | | (SEQ-ID NOs.: 50, 51) | |
| 7 | Pep 16 + 21 | Pep-16, Pep-21 | see FIG. 8 and FIG. 9 |
| | | (SEQ-ID NOs.: 53, 58) | |
| 8 | Pep 6 | Pep-6 (SEQ-ID NO.: 43) | see FIG. 8 and FIG. 9 |
| 9 | Pep 7 | Pep-7 (SEQ-ID NO.: 44) | see FIG. 8 and FIG. 9 |
| 10 | Pep 8 | Pep-8 (SEQ-ID NO.: 45) | see FIG. 5 and FIG. 6 |
| 11 | Pep 9 | Pep-9 (SEQ-ID NO.: 46) | see FIG. 8 and FIG. 9 |
| 12 | Pep 15 | Pep-15 (SEQ-ID NO.: 52) | see FIG. 8 and FIG. 9 |
| 13 | Pep 20 | Pep-20 (SEQ-ID NO.: 57) | see FIG. 8 and FIG. 9 |
| 14 | Pep all | SEQ ID NOs: 43, 44, 46, 47, | 12 polypeptides, see FIG. 8 and |
| | | 50-58 | FIG. 9 |

FIG. 5 shows the results of CD8 activities: a mixture containing 12 antigen polypeptides showed a large immune response, and the group containing Pep 6+7+9+10 showed an almost equivalent immune response. This proves the efficacy of the composition.

The efficacy of individual antigen polypeptides is shown in FIGS. 8 and 9, where the single peptides Pep-6 and -7 showed some CD4 response and a strong CD8+ response, while the two-component compositions Pep-13+14 and Pep-16+21 showed an intermediate CD4 response, while the composition (Pep all) containing 12 antigen polypeptides showed a good response (see FIG. 8).

Pep-6 and Pep-7 showed high individual activities in the ICS study. These peptides are overlapping tandem peptides, Pep-6 (SEQ ID NO.: 43, KFQPTNGVGYQPYRVVLSFELLHAP) with the HLA sequences linked to the SARS-COV-2 epitope FQPTNGVGY (SEQ ID NO.: 68), GYQPYRVV (SEQ ID NO . . . 70), PYRVVLSF (SEQ ID NO.: 78) and Pep-7 (SEQ ID NO.: 44, MAYRFNGIGVTQNVLYE) with the HLA sequences AYRFNGIGV (SEQ ID NO.: 61) and IGVTQNVLY (SEQ ID NO.: 71) matching SARS-COV-2. They are included in Group No. 4 (Table E7-1), thus demonstrating that an overlapping tandem antigen polypeptide according to the present invention can elicit an immunogenic response and that a combination of at least four antigen polypeptides (Group No. 4) containing overlapping tandem peptides is highly efficient in eliciting a robust CD8+ response in ICS experiments.

A combination of Pep-6 and Pep-7, two antigen polypeptides according to the present invention, would result in an effective immunogenic response and would therefore provide a pharmacological composition that would be very effective for treating subjects or individuals who have contracted a viral infection or could be used prophylactically in such subjects who are at risk of contracting such infection.

1-35. (canceled)

36. A pharmaceutical composition for use as a medicament in the treatment of a subject or group of subjects suffering from or at risk of suffering from a viral infectious disease, comprising
2 to 12 antigen polypeptide sequences each comprising two different HLA-A and/or HLA-B antigen peptides, wherein the HLA antigen peptide corresponds to at least one amino acid sequence of at least one virus transcriptome corresponding to a part of a viral epitope, in a pharmacologically effective amount, wherein the pharmacologically effective amount of each individual antigen polypeptide in the composition is in an absolute concentration ranging from 100 to 1,000 μg, wherein the antigen polypeptides have an amino acid length between 9 and 40,
characterized in that
the antigen polypeptide sequence comprises the following scaffold amino acid sequence:
an overlapping tandem peptide, i.e., a compound, construct, or polypeptide, wherein the amino acid sequence of which comprises one HLA-A antigen peptide or HLA-B antigen peptide as defined herein, wherein the amino acid sequence comprises, in addition to the HLA-A antigen peptide or HLA-B antigen peptide, up to 1 to 30 amino acids (long peptide), which comprises or consists of 2 different HLA-A and/or HLA-B antigen peptide sequences, wherein the amino acid sequence of both comprise one HLA-A antigen peptide or HLA-B antigen peptide as defined herein, wherein the amino acid sequence comprises, in addition to the HLA-A antigen peptide or HLA-B antigen peptide, up to 1 to 30 amino acids (long peptide); and/or, wherein the HLA-A antigen peptide or HLA-B antigen peptide as defined herein having at least 90% sequence identity or similarity to the native HLA antigen peptide (similarity peptide); and/or wherein the HLA-A antigen peptide or HLA-B antigen peptide as defined herein having an amino acid sequence consisting of only one amino acid substitution relative to the amino acid sequence of the native HLA antigen peptide (substitution peptide), in which two HLA antigen peptides overlap in their amino acid sequence.

37. The pharmaceutical composition for use as a medicament in the treatment of a subject or group of subjects according to claim 36, wherein the antigen polypeptide corresponding to MHC class I complexes is selected to match a sequence of the viral epitope of at least two different viral infectious diseases.

38. The pharmaceutical composition for use as a medicament in the treatment of a subject or group of subjects according to claim 36, where at least one antigen polypeptide of the pharmaceutical composition is an overlapping polypeptide consisting of two amino acid sequences from the epitopes of at least two different viral infectious diseases.

39. The pharmaceutical composition for use as a medicament in the treatment of a subject or group of subjects according to claim 36, wherein six up to twelve different pharmacological active antigen polypeptides comprising of HLA antigen peptides in accordance with the current invention and matching the epitope of at least one viral infectious disease.

40. The pharmaceutical composition for use as a medicament in the treatment of a subject or group of subjects according to claim 36, wherein the composition is used in the prophylaxis and/or treatment of a SARS-COV-2 infectious disease.

41. The pharmaceutical composition for use as a medicament in the treatment of a subject or group of subjects according to claim 40, wherein the HLA-A and/or HLA-B antigen peptide is an amino acid sequence selected from the group consisting of SEQ ID NO: 2-37, 59-79.

42. The pharmaceutical composition for use as a medicament in the treatment of a subject or group of subjects according to claim 40, wherein the antigen polypeptide is an amino acid sequence selected from the group consisting of SEQ ID NOs 39, 40, 42-58, and/or 80-90.

43. The pharmaceutical composition for use as a medicament in the treatment of a subject or group of subjects according to claim 36, wherein the pharmaceutical composition comprises a pharmaceutically acceptable carrier liquid and a pharmaceutically acceptable adjuvant, carrier, diluent and/or excipient.

44. The pharmaceutical composition for use as a medicament in the treatment of a subject or group of subjects according to claim 36, wherein the pharmaceutical composition is applied subcutaneously, intramuscularly or intradermally.

45. The pharmaceutical composition for use as a medicament in the treatment of a subject or group of subjects according to claim 44, wherein said subject is a human.

46. An antigen polypeptide in a pharmaceutical composition for use as a medicament in the treatment of a subject or group of subjects according to claim 36 comprising two different HLA-A and/or HLA-B antigen peptides, wherein the HLA antigen peptide corresponds to at least one amino acid sequence of at least one virus transcriptome corresponding to a part of a viral epitope, wherein the antigen polypeptides have an amino acid amino acid length between 9 and 40,
wherein the antigen polypeptide sequence comprises the following scaffold amino acid sequence:
an overlapping tandem peptide, i.e., a compound, construct, or polypeptide, wherein the amino acid sequence of which comprises one HLA-A antigen peptide or HLA-B antigen peptide as defined herein, wherein the amino acid sequence comprises, in addition to the HLA-A antigen peptide or HLA-B antigen peptide, up to 1 to 30 amino acids (long peptide), which comprises or consists of 2 different HLA-A and/or HLA-B antigen peptide sequences, wherein the amino acid sequence of both comprise one HLA-A antigen peptide or HLA-B antigen peptide as defined herein, wherein the amino acid sequence comprises, in addition to the HLA-A antigen peptide or HLA-B antigen peptide, up to 1 to 30 amino acids (long peptide); and/or wherein the HLA-A antigen peptide or HLA-B antigen peptide as defined herein having at least 90% sequence identity or similarity to the native HLA antigen peptide (similarity peptide); and/or wherein the HLA-A antigen peptide or HLA-B antigen peptide as defined herein having an amino acid sequence consisting of only one amino acid substitution relative to the amino acid sequence of the native HLA antigen peptide (substitution peptide), in which the at least two HLA antigen peptides overlap in their amino acid sequence; and/or
an overlapping tandem peptide, i.e. a compound, construct, or polypeptide, wherein the amino acid sequence of which is as defined in item (a), comprising at least 2 different HLA-A and/or HLA-B antigen peptide sequences, both as defined in item (a), wherein the amino acid sequences match viral epitopes from at least two different viral infectious diseases.

47. The antigen polypeptide for use as a medicament in the treatment of a subject or group of subjects according to claim 46, wherein the antigen polypeptide in accordance with the current invention is an overlapping polypeptide containing between 2 to 10 different amino acid sequences (HLA antigen peptides) matching a viral epitope corresponding to MHC Class I and/or MHC Class II complexes.

48. The antigen polypeptide for use as a medicament in the treatment of a subject or group of subjects according to claim 46, wherein the antigen polypeptide is selected from the group consisting of the amino acid sequences set forth in SEQ ID NOs. 39, 40, 42-58; 80-90.

49. The antigen polypeptide for use as a medicament in the treatment of a subject or group of subjects according to claim 46 for use as a medicament, wherein the method comprising administering to the subject a treatment regimen including a pharmacologically effective amount of the antigen polypeptide.

50. The antigen polypeptide for use as a medicament in the treatment of a subject or group of subjects according to claim 46 that are immunogenic in the patient or group of patients, determined by an immunogenicity assay.

51. An in vitro method for determining at least one antigen polypeptide according to claim 46, comprising the following steps:
a) determining the amino acid sequence(s) from at least one viral genome from virus sequencing proteins containing viral epitopes presented on the viral surface,
b) comparing the amino acid sequence(s) determined according to (a) with those of at least one other related virus and determining the conserved regions in the viral genomes,
c) determining amino acid sequence(s) corresponding to MHC class I and/or class II complexes and/or matching to B-cells belonging to conserved viral epitope region(s) determined according to (b),
d) developing synthetic antigen polypeptide(s) containing the amino acid sequences selected according to (c) and determining the physiological and physicochemical properties,
e) creating a composition of at least one and up to twelve antigen polypeptide(s) according to (d) while considering HLA allele distribution of the worldwide population.

52. The method of claim 51, whereas the viral epitopes are determined from at least two infectious disease(s).

53. The method of claim 51, where the synthetic polypeptides created in step (d) are antigen polypeptides consisting of overlapping antigen peptides corresponding to MHC I and/or MHC II complexes and matching B-cell and T-cell epitopes.

54. The method of the claim 51, where the antigen polypeptides of the combination preparation cover more than 95% of the HLA alleles of the worldwide human population.

55. A method for preparing a pharmaceutical composition for use as a medicament in the treatment of a subject or group of subjects according to claim 36, comprising the following steps:
a) determining at least two and up to twelve antigen polypeptide corresponding to MHC class I complexes matching at least two viral epitopes according to the method of claim 51, which are matching at least 98% of the most common HLA alleles distributed in the worldwide population;
b) synthesizing the antigen polypeptide(s) corresponding to MHC class I complexes determined in step (a);
c) preparing the pharmaceutical composition according to the invention comprising at least one antigen polypeptide(s) corresponding to MHC class I complexes matching at least one viral epitope in a suitable pharmaceutical formulation.


SEQUENCE LISTING (90 sequences)

SEQ ID NO: 1 - protein, 9 aa, Severe acute respiratory syndrome coronavirus 2
LLFNKVTLA

SEQ ID NO: 2 - protein, 9 aa, Severe acute respiratory syndrome coronavirus 2
FIKQYGDCL

SEQ ID NO: 3 - protein, 9 aa, Severe acute respiratory syndrome coronavirus 2
TLADAGFIK

SEQ ID NO: 4 - protein, 9 aa, Severe acute respiratory syndrome coronavirus 2
KCYGVSPTK

SEQ ID NO: 5 - protein, 9 aa, Severe acute respiratory syndrome coronavirus 2
CYGVSPTKL

SEQ ID NO: 6 - protein, 9 aa, Severe acute respiratory syndrome coronavirus 2
RLFRKSNLK

SEQ ID NO: 7 - protein, 9 aa, Severe acute respiratory syndrome coronavirus 2
NYNYLYRLF

SEQ ID NO: 8 - protein, 9 aa, Severe acute respiratory syndrome coronavirus 2
YRLFRKSNL

SEQ ID NO: 9 - protein, 9 aa, Severe acute respiratory syndrome coronavirus 2
PYRVVVLSF

SEQ ID NO: 10 - protein, 9 aa, Severe acute respiratory syndrome coronavirus 2
YQPYRVVVL

SEQ ID NO: 11 - protein, 9 aa, Severe acute respiratory syndrome coronavirus 2
RVVVLSFEL

SEQ ID NO: 12 - protein, 9 aa, Severe acute respiratory syndrome coronavirus 2
YRFNGIGVT

SEQ ID NO: 13 - protein, 9 aa, Severe acute respiratory syndrome coronavirus 2
FPRGQGVPI

SEQ ID NO: 14 - protein, 9 aa, Severe acute respiratory syndrome coronavirus 2
KPRQKRTAT

SEQ ID NO: 15 - protein, 9 aa, Severe acute respiratory syndrome coronavirus 2
KAYNVTQAF

SEQ ID NO: 16 - protein, 9 aa, Severe acute respiratory syndrome coronavirus 2
GMSRIGMEV

SEQ ID NO: 17 - protein, 9 aa, Severe acute respiratory syndrome coronavirus 2
MEVTPSGTW

SEQ ID NO: 18 - protein, 9 aa, Severe acute respiratory syndrome coronavirus 2
ASAFFGMSR

SEQ ID NO: 19 - protein, 9 aa, Severe acute respiratory syndrome coronavirus 2
FAPSASAFF

SEQ ID NO: 20 - protein, 9 aa, Severe acute respiratory syndrome coronavirus 2
AQFAPSASA

SEQ ID NO: 21 - protein, 9 aa, Severe acute respiratory syndrome coronavirus 2
VSLVKPSFY

SEQ ID NO: 22 - protein, 9 aa, Severe acute respiratory syndrome coronavirus 2
SLVKPSFYV

SEQ ID NO: 23 - protein, 9 aa, Severe acute respiratory syndrome coronavirus 2
SFYVYSRVK

SEQ ID NO: 24 - protein, 9 aa, Severe acute respiratory syndrome coronavirus 2
LVKPSFYVY

SEQ ID NO: 25 - protein, 9 aa, Severe acute respiratory syndrome coronavirus 2
NSSRVPDLL

SEQ ID NO: 26 - protein, 9 aa, Severe acute respiratory syndrome coronavirus 2
YVYSRVKNL

SEQ ID NO: 27 - protein, 9 aa, Severe acute respiratory syndrome coronavirus 2
VLNDILSRL

SEQ ID NO: 28 - protein, 9 aa, Severe acute respiratory syndrome coronavirus 2
ISSVLNDIL

SEQ ID NO: 29 - protein, 9 aa, Severe acute respiratory syndrome coronavirus 2
FSASTSAFV

SEQ ID NO: 30 - protein, 9 aa, Severe acute respiratory syndrome coronavirus 2
ILASFSAST

SEQ ID NO: 31 - protein, 9 aa, Severe acute respiratory syndrome coronavirus 2
SFSASTSAF

SEQ ID NO: 32 - protein, 9 aa, Severe acute respiratory syndrome coronavirus 2
AARVVRSIF

SEQ ID NO: 33 - protein, 9 aa, Severe acute respiratory syndrome coronavirus 2
SLPFGWLIV

SEQ ID NO: 34 - protein, 9 aa, Severe acute respiratory syndrome coronavirus 2
IQASLPFGW

SEQ ID NO: 35 - protein, 9 aa, Severe acute respiratory syndrome coronavirus 2
ASLPFGWLI

SEQ ID NO: 36 - protein, 9 aa, Severe acute respiratory syndrome coronavirus 2
RIAGHHLGR

SEQ ID NO: 37 - protein, 9 aa, Severe acute respiratory syndrome coronavirus 2
HLRIAGHHL

SEQ ID NO: 38 - protein, 26 aa, synthetic construct
  source 1..26: note = synthetic peptide used as antigen comprising at least one HLA antigen peptide
LLFNKVTLADAGFIKQYGDCLGDIAA

SEQ ID NO: 39 - protein, 19 aa, synthetic construct
  source 1..19: note = synthetic peptide used as antigen comprising at least one HLA antigen peptide
KCYGVSPTKLNDLCFTNVY

SEQ ID NO: 40 - protein, 26 aa, synthetic construct
  source 1..26: note = synthetic peptide used as antigen comprising at least one HLA antigen peptide
LDSKVGGNYNYLYRLFRKSNLKPFER

SEQ ID NO: 41 - protein, 9 aa, synthetic construct
  source 1..9: note = synthetic peptide used as antigen comprising at least one HLA antigen peptide
FPRGQGVPI

SEQ ID NO: 42 - protein, 30 aa, synthetic construct
  source 1..30: note = synthetic peptide used as antigen comprising at least one HLA antigen peptide
SAAEASKKPRQKRTATKAYNVTQAFGRRPE

SEQ ID NO: 43 - protein, 26 aa, synthetic construct
  source 1..26: note = synthetic peptide used as antigen comprising at least one HLA antigen peptide
KFQPTNGVGYQPYRVVVLSFELLHAP

SEQ ID NO: 44 - protein, 17 aa, synthetic construct
  source 1..17: note = synthetic peptide used as antigen comprising at least one HLA antigen peptide
MAYRFNGIGVTQNVLYE

SEQ ID NO: 45 - protein, 27 aa, synthetic construct
  source 1..27: note = synthetic peptide used as antigen comprising at least one HLA antigen peptide
VSLVKPSFYVYSRVKNLNSSRVPDLLV

SEQ ID NO: 46 - protein, 17 aa, synthetic construct
  source 1..17: note = synthetic peptide used as antigen comprising at least one HLA antigen peptide
AATKMSECVLGQSKRVD

SEQ ID NO: 47 - protein, 17 aa, synthetic construct
  source 1..17: note = synthetic peptide used as antigen comprising at least one HLA antigen peptide
AISSVLNDILSRLDKVE

SEQ ID NO: 48 - protein, 17 aa, synthetic construct
  source 1..17: note = synthetic peptide used as antigen comprising at least one HLA antigen peptide
FFGMSRIGMEVTPSGTW

SEQ ID NO: 49 - protein, 15 aa, synthetic construct
  source 1..15: note = synthetic peptide used as antigen comprising at least one HLA antigen peptide
AFASEAARVVRSIFS

SEQ ID NO: 50 - protein, 17 aa, synthetic construct
  source 1..17: note = synthetic peptide used as antigen comprising at least one HLA antigen peptide
TFEYVSQPFLMDLEGKQ

SEQ ID NO: 51 - protein, 14 aa, synthetic construct
  source 1..14: note = synthetic peptide used as antigen comprising at least one HLA antigen peptide
GDCLGDIAARDLIC

SEQ ID NO: 52 - protein, 21 aa, synthetic construct
  source 1..21: note = synthetic peptide used as antigen comprising at least one HLA antigen peptide
LGKYEQYIKWPWYIWLGFIAG

SEQ ID NO: 53 - protein, 20 aa, synthetic construct
  source 1..20: note = synthetic peptide used as antigen comprising at least one HLA antigen peptide
EVAKNLNESLIDLQELGKYE

SEQ ID NO: 54 - protein, 16 aa, synthetic construct
  source 1..16: note = synthetic peptide used as antigen comprising at least one HLA antigen peptide
DGYFKIYSKHTPINLV

SEQ ID NO: 55 - protein, 20 aa, synthetic construct
  source 1..20: note = synthetic peptide used as antigen comprising at least one HLA antigen peptide
GKEDLKFPRGQGVPINTNSS

SEQ ID NO: 56 - protein, 15 aa, synthetic construct
  source 1..15: note = synthetic peptide used as antigen comprising at least one HLA antigen peptide
RQGTDYKHWPQIAQF

SEQ ID NO: 57 - protein, 15 aa, synthetic construct
  source 1..15: note = synthetic peptide used as antigen comprising at least one HLA antigen peptide
KDLSPRWYFYYLGTG

SEQ ID NO: 58 - protein, 17 aa, synthetic construct
  source 1..17: note = synthetic peptide used as antigen comprising at least one HLA antigen peptide
IRAAEIRASANLAATKM

SEQ ID NO: 59 - protein, 9 aa, Severe acute respiratory syndrome coronavirus 2
AEIRASANL

SEQ ID NO: 60 - protein, 9 aa, Severe acute respiratory syndrome coronavirus 2
ASANLAATK

SEQ ID NO: 61 - protein, 9 aa, Severe acute respiratory syndrome coronavirus 2
DCLGDIAAR

SEQ ID NO: 62 - protein, 9 aa, Severe acute respiratory syndrome coronavirus 2
AYRFNGIGV

SEQ ID NO: 63 - protein, 9 aa, Severe acute respiratory syndrome coronavirus 2
DGYFKIYSK

SEQ ID NO: 64 - protein, 9 aa, Severe acute respiratory syndrome coronavirus 2
DILSRLDKV

SEQ ID NO: 65 - protein, 9 aa, Severe acute respiratory syndrome coronavirus 2
DLSPRWYFY

SEQ ID NO: 66 - protein, 9 aa, Severe acute respiratory syndrome coronavirus 2
EYVSQPFLM

SEQ ID NO: 67 - protein, 9 aa, Severe acute respiratory syndrome coronavirus 2
FPRGQGVPI

SEQ ID NO: 68 - protein, 9 aa, Severe acute respiratory syndrome coronavirus 2
FQPTNGVGY

SEQ ID NO: 69 - protein, 9 aa, Severe acute respiratory syndrome coronavirus 2
GYFKIYSKH

SEQ ID NO: 70 - protein, 9 aa, Severe acute respiratory syndrome coronavirus 2
GYQPYRVVV

SEQ ID NO: 71 - protein, 9 aa, Severe acute respiratory syndrome coronavirus 2
IGVTQNVLY

SEQ ID NO: 72 - protein, 9 aa, Severe acute respiratory syndrome coronavirus 2
KDLSPRWYF

SEQ ID NO: 73 - protein, 9 aa, Severe acute respiratory syndrome coronavirus 2
KHWPQIAQF

SEQ ID NO: 74 - protein, 9 aa, Severe acute respiratory syndrome coronavirus 2
KIYSKHTPI

SEQ ID NO: 75 - protein, 9 aa, Severe acute respiratory syndrome coronavirus 2
LSPRWYFYY

SEQ ID NO: 76 - protein, 9 aa, Severe acute respiratory syndrome coronavirus 2
NGVGYQPYR

SEQ ID NO: 77 - protein, 9 aa, Severe acute respiratory syndrome coronavirus 2
NLNESLIDL

SEQ ID NO: 78 - protein, 9 aa, Severe acute respiratory syndrome coronavirus 2
PYRVVVLSF

SEQ ID NO: 79 - protein, 9 aa, Severe acute respiratory syndrome coronavirus 2
QYIKWPWYI

SEQ ID NO: 80 - protein, 27 aa, synthetic construct
LGKYEQYIKWPWYIWLGFIAGLIAIVM

SEQ ID NO: 81 - protein, 17 aa, synthetic construct
LGKYEQYIKWPWYIWLG

SEQ ID NO: 82 - protein, 14 aa, synthetic construct
KIYSKHTPINLVRD

SEQ ID NO: 83 - protein, 14 aa, synthetic construct
LSRLDKVEAEVQID

SEQ ID NO: 84 - protein, 12 aa, synthetic construct
LGQSKRVDFCGK

SEQ ID NO: 85 - protein, 18 aa, synthetic construct
DGYFKIYSKHTPINLVRD

SEQ ID NO: 86 - protein, 14 aa, synthetic construct
QYIKWPWYIWLGFI

SEQ ID NO: 87 - protein, 16 aa, synthetic construct
VKQLSSNFGAISSVLN

SEQ ID NO: 88 - protein, 14 aa, synthetic construct
KQLSSNFGAISSVL

SEQ ID NO: 89 - protein, 33 aa, synthetic construct
AEIRASANLAATKMSECVLGQSKRVDFCGKGYH

SEQ ID NO: 90 - protein, 27 aa, synthetic construct
AEIRASANLAATKMSECVLGQSKRVDF